SEQUENCE LISTING (85 sequences)

SEQ ID NO: 1 - protein, 5 aa, Mus musculus
TYAMN

SEQ ID NO: 2 - protein, 19 aa, Mus musculus
RIRSKYNNYATYYADSVKG

SEQ ID NO: 3 - protein, 14 aa, Mus musculus
HGNFGNSYVSWFAY

SEQ ID NO: 4 - protein, 14 aa, Mus musculus
GSSTGAVTTSNYAN

SEQ ID NO: 5 - protein, 7 aa, Mus musculus
GTNKRAP

SEQ ID NO: 6 - protein, 9 aa, Mus musculus
ALWYSNLWV

SEQ ID NO: 7 - protein, 125 aa, Mus musculus
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 8 - protein, 109 aa, Mus musculus
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 9 - protein, 116 aa, Artificial Sequence
  83A10 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSS

SEQ ID NO: 10 - protein, 116 aa, Artificial Sequence
  Mab21 VH Mab22 VH Mab42 VH
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSS

SEQ ID NO: 11 - protein, 109 aa, Artificial Sequence
  83A10 VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIK

SEQ ID NO: 12 - protein, 109 aa, Artificial Sequence
  Mab21 VL, Mab27 VL, Mab33 VL, Mab39 VL
EIVLTQSPGTLSLSPGERATLSCRASQSVSEYYLAWYQQKPGQAPRLLIEHASTRATGIPDRFSGSGSGTDFTLTISRLEPEDFAVYYCQQYGYPPDFTFGQGTKVEIK

SEQ ID NO: 13 - protein, 109 aa, Artificial Sequence
  Mab22 VL
EIVLTQSPGTLSLSPGERATLSCRASQSVSSYYLAWYQQKPGQAPRLLISGAGSRATGIPDRFSGSGSGTDFTLTISRLEPEDFAVYYCQQYGYPPDFTFGQGTKVEIK

SEQ ID NO: 14 - protein, 109 aa, Artificial Sequence
  Mab42 VL
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIK

SEQ ID NO: 15 - protein, 5 aa, Artificial Sequence
  83A10 CDR1H
SYAMS

SEQ ID NO: 16 - protein, 17 aa, Artificial Sequence
  83A10 CDR2H
AISGSGGSTYYADSVKG

SEQ ID NO: 17 - protein, 7 aa, Artificial Sequence
  83A10 CDR3H, Mab21 CDR3H, Mab22 CDR3H, Mab42 CDR3H, Mab 27 CDR3H, Mab33 CDR3H, Mab39 CDR3H
VLGWFDY

SEQ ID NO: 18 - protein, 12 aa, Artificial Sequence
  83A10 CDR1L
RASQSVSSSYLA

SEQ ID NO: 19 - protein, 7 aa, Artificial Sequence
  83A10 CDR2L
GASSRAT

SEQ ID NO: 20 - protein, 10 aa, Artificial Sequence
  83A10 CDR3L, Mab21 CDR3L, Mab22 CDR3L, Mab42 CDR3L
QQYGYPPDFT

SEQ ID NO: 21 - protein, 5 aa, Artificial Sequence
  Mab21 CDR1H Mab22 CDR1H Mab42 CDR1H
DNAMG

SEQ ID NO: 22 - protein, 17 aa, Artificial Sequence
  Mab21 CDR2H Mab22 CDR2H Mab42 CDR2H
AISGPGSSTYYADSVKG

SEQ ID NO: 23 - protein, 13 aa, Artificial Sequence
  Mab21 CDR1L
RASQSVSEYYLAW

SEQ ID NO: 24 - protein, 8 aa, Artificial Sequence
  Mab21 CDR2L
EHASTRAT

SEQ ID NO: 25 - protein, 12 aa, Artificial Sequence
  Mab22 CDR1L
RASQSVSSYYLA

SEQ ID NO: 26 - protein, 7 aa, Artificial Sequence
  Mab22 CDR2L
GAGSRAT

SEQ ID NO: 27 - protein, 12 aa, Artificial Sequence
  Mab42 CDR1L
RASQSVSDEYLS

SEQ ID NO: 28 - protein, 7 aa, Artificial Sequence
  Mab42 CDR2L
SASTRAT

SEQ ID NO: 29 - protein, 5 aa, Artificial Sequence
  Mab27 CDR1H
SAPMG

SEQ ID NO: 30 - protein, 16 aa, Artificial Sequence
  Mab27 CDR2H
AISYIGHTYYADSVKG

SEQ ID NO: 31 - protein, 12 aa, Artificial Sequence
  Mab27 CDR1L Mab33 CDR1L Mab39 CDR1L
RASQSVSEYYLA

SEQ ID NO: 32 - protein, 7 aa, Artificial Sequence
  Mab27 CDR2L Mab33 CDR2L Mab39 CDR2L
HASTRAT

SEQ ID NO: 33 - protein, 10 aa, Artificial Sequence
  Mab27 CDR3L Mab33 CDR3L Mab39 CDR3L
QQYGYPPDFT

SEQ ID NO: 34 - protein, 5 aa, Artificial Sequence
  Mab33 CDR1H
TNAMG

SEQ ID NO: 35 - protein, 17 aa, Artificial Sequence
  Mab33 CDR2H
AINRFGGSTYYADSVKG

SEQ ID NO: 36 - protein, 5 aa, Artificial Sequence
  Mab39 CDR1H
QNAMG

SEQ ID NO: 37 - protein, 17 aa, Artificial Sequence
  Mab39 CDR2H
AISPTGFSTYYADSVKG

SEQ ID NO: 38 - protein, 115 aa, Artificial Sequence
  Mab27 VH
EVQLLESGGGLVQPGGSLRLSCAASGFTFSSAPMGWVRQAPGKGLEWVSAISYIGHTYYADSVKGRFTISRDNSKNTLYLQMNSLRAEDTAVYYCAKVLGWFDYWGQGTLVTVSS

SEQ ID NO: 39 - protein, 116 aa, Artificial Sequence
  Mab33 VH
EVQLLESGGGLVQPGGSLRLSCAASGFTFYTNAMGWVRQAPGKGLEWVSAINRFGGSTYYADSVKGRFTISRDNSKNTLYLQMNSLRAEDTAVYYCAKVLGWFDYWGQGTLVTVSS

SEQ ID NO: 40 - protein, 116 aa, Artificial Sequence
  Mab39 VH
EVQLLESGGGLVQPGGSLRLSCAASGFTFTQNAMGWVRQAPGKGLEWVSAISPTGFSTYYADSVKGRFTISRDNSKNTLYLQMNSLRAEDTAVYYCAKVLGWFDYWGQGTLVTVSS

SEQ ID NO: 41 - protein, 103 aa, Artificial Sequence
  83A10 BCMA CH1, Mab21 BCMA CH1, Mab22 BCMA CH1, Mab42 BCMA CH1
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 42 - protein, 107 aa, Artificial Sequence
  83A10 BCMA CL, Mab21 BCMA CL, Mab22 BCMA CL, Mab42 BCMA CL
RTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 103 aa, Artificial Sequence
  CD3 CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 44 - protein, 107 aa, Artificial Sequence
  CD3 CL
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 671 aa, Artificial Sequence
  83A10-TCBcv knob HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 446 aa, Artificial Sequence
  83A10-TCBcv hole HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 47 - protein, 216 aa, Artificial Sequence
  83A10-TCBcv LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 232 aa, Artificial Sequence
  CD3 LC
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 671 aa, Artificial Sequence
  21-TCBcv knob HC
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 446 aa, Artificial Sequence
  21-TCBcv hole HC
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 216 aa, Artificial Sequence
  21-TCBcv LC
EIVLTQSPGTLSLSPGERATLSCRASQSVSEYYLAWYQQKPGQAPRLLIEHASTRATGIPDRFSGSGSGTDFTLTISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 671 aa, Artificial Sequence; identical to SEQ ID NO: 49
  22-TCBcv knob HC

SEQ ID NO: 53 - protein, 446 aa, Artificial Sequence; identical to SEQ ID NO: 50
  22-TCBcv hole HC

SEQ ID NO: 54 - protein, 216 aa, Artificial Sequence
  22-TCBcv LC
EIVLTQSPGTLSLSPGERATLSCRASQSVSSYYLAWYQQKPGQAPRLLISGAGSRATGIPDRFSGSGSGTDFTLTISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 671 aa, Artificial Sequence; identical to SEQ ID NO: 49
  42-TCBcv knob HC

SEQ ID NO: 56 - protein, 446 aa, Artificial Sequence; identical to SEQ ID NO: 50
  42-TCBcv hole HC

SEQ ID NO: 57 - protein, 216 aa, Artificial Sequence
  42-TCBcv LC
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 13 aa, Artificial Sequence
  83A10 CDR1L
RASQSVSSSYLAW

SEQ ID NO: 59 - protein, 8 aa, Artificial Sequence
  83A10 CDR2L
YGASSRAT

SEQ ID NO: 60 - protein, 13 aa, Artificial Sequence
  Mab22 CDR1L
RASQSVSSYYLAW

SEQ ID NO: 61 - protein, 8 aa, Artificial Sequence
  Mab22 CDR2L
SGAGSRAT

SEQ ID NO: 62 - protein, 13 aa, Artificial Sequence
  Mab42 CDR1L
RASQSVSDEYLSW

SEQ ID NO: 63 - protein, 8 aa, Artificial Sequence
  Mab42 CDR2L
HSASTRAT

SEQ ID NO: 64 - protein, 5 aa, Artificial Sequence
  C11D5.3 CDR1H?
DYSIN

SEQ ID NO: 65 - protein, 17 aa, Artificial Sequence
  C11D5.3 CDR2H?
WINTETREPAYAYDFRG

SEQ ID NO: 66 - protein, 8 aa, Artificial Sequence
  C11D5.3 CDR3H?
DYSYAMDY

SEQ ID NO: 67 - protein, 15 aa, Artificial Sequence
  C11D5.3 CDR1 L1?
RASESVSVIGAHLIH

SEQ ID NO: 68 - protein, 7 aa, Artificial Sequence
  C11D5.3 CDR2? L1
LASNLET

SEQ ID NO: 69 - protein, 9 aa, Artificial Sequence
  C11D5.3 CDR3 L1?
LQSRIFPRT

SEQ ID NO: 70 - protein, 15 aa, Artificial Sequence
  C11D5.3 CDR1 L2?
RASESVDNYGFSFMH

SEQ ID NO: 71 - protein, 7 aa, Artificial Sequence
  C11D5.3 CDR2 L2?
RASNLES

SEQ ID NO: 72 - protein, 9 aa, Artificial Sequence
  C11D5.3 CDR3 L2?
QQSNKDPRT

SEQ ID NO: 73 - protein, 15 aa, Artificial Sequence
  C11D5.3 CDR1 L3?
RASESVTILGSHLIH

SEQ ID NO: 74 - protein, 7 aa, Artificial Sequence
  C11D5.3 CDR2 L3?
LASNVQT

SEQ ID NO: 75 - protein, 9 aa, Artificial Sequence
  C11D5.3 CDR3 L3?
LQSRTIPRT

SEQ ID NO: 76 - protein, 117 aa, Artificial Sequence
  C11D5.3 VH?
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 77 - protein, 111 aa, Artificial Sequence
  C11D5.3 VL1?
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 78 - protein, 111 aa, Artificial Sequence
  C11D5.3 VL2?
DIVLTQSPASLAVSLGQRATISCRASESVDNYGFSFMHWYQQKPGQPPKLLIYRASNLESGIPARFSGSGSRTDFALTINPVETDDVATYYCQQSNKDPRTFGGGTKLEIK

SEQ ID NO: 79 - protein, 111 aa, Artificial Sequence
  C11D5.3 VL3?
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 80 - protein, 122 aa, Artificial Sequence
  A7D12.2 VH
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 81 - protein, 107 aa, Artificial Sequence
  A7D12.2 VL
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 82 - protein, 117 aa, Artificial Sequence
  C12A32.2 VH
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 83 - protein, 111 aa, Artificial Sequence
  C12A32.2 VL
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 84 - protein, 117 aa, Artificial Sequence
  C13F12.1 VH
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSS

SEQ ID NO: 85 - protein, 111 aa, Artificial Sequence; identical to SEQ ID NO: 83
  C13F12.1 VL

(19) United States
(12) Patent Application Publication
(10) Pub. No.: US 2023/0172923 A1
(43) Pub. Date: Jun. 8, 2023

(54) METHODS OF TREATING CYTOKINE-RELATED ADVERSE EVENTS

(71) Applicant: BRISTOL-MYERS SQUIBB COMPANY, Princeton, NJ (US)

(72) Inventors: Danny Vijey JEYARAJU, Hillsborough, NJ (US); Michael AMATANGELO, Madison, NJ (US); Ross LA MOTTE-MOHS, Randolph, NJ (US); William PIERCEALL, Madison, NJ (US); Anjan G. THAKURTA, Basking Ridge, NJ (US)

(21) Appl. No.: 17/921,893

(22) PCT Filed: Apr. 29, 2021
(86) PCT No.: PCT/US2021/029877

§ 371 Date: Oct. 27, 2022

(30) Foreign Application Priority Data
Apr. 30, 2020 (EP) 20172322.8

(51) Int. Cl. A61K 31/4545; A61P 37/06

(52) U.S. Cl. CPC A61K 31/4545; A61P 37/06

(57) ABSTRACT

The disclosure relates to agents for use in the treatment or prevention of a cytokine-related adverse event or disease, such as cytokine release syndrome (CRS).

### FIELD OF THE INVENTION

The present invention relates to agents for use in the treatment or prevention of a cytokine-related adverse event or disease, such as cytokine release syndrome (CRS).

### BACKGROUND

Cytokine release syndrome (CRS) is a potentially severe and life-threatening adverse event that is characterised by elevated levels of pro-inflammatory cytokines, in severe cases resulting in a systemic inflammatory response. Without being bound by theory, CRS may result from a large and/or rapid secretion of cytokines, for example because of activation and/or proliferation of immune effector cells. For example, CRS occurs when large numbers of white blood cells are activated and release inflammatory cytokines.

CRS represents one of the most frequent serious adverse effects of T cell-engaging immunotherapies, including bispecific T-cell engaging antibodies and CAR T-cells (Teachey et al. (2013) Blood. 121(26): 5154-5157; Hay et al. (2017) Blood. 130(21): 2295-2306). CRS has also been described after infusion of several antibody-based therapies (Chatenoud et al. (1990) Transplantation. 49(4): 697-702; Freeman et al. (2015) Blood. 126(24): 2646-2649; Suntharalingam et al. (2006) N. Engl. J. Med. 355(10): 1018-1028; Winkler et al. (1999) Blood. 94(7): 2217-2224). The adverse event can also be triggered by other cell therapies and immunotherapies, as well as infection.

Although treatments to mitigate the symptoms of the cytokine release exist, such as Tocilizumab and corticosteroids, understanding and eliminating the source of the cytokine release could potentially increase the therapeutic index of these cell therapies and immunotherapies. Many cytokines show elevated levels in serum of patients with CRS including interleukin-6 (IL-6). interleukin-10 (IL-10), and IL1-beta (Norelli et al. (2018) Nat. Med. 24(6): 739-748; Wang and Han (2018) Biomark. Res. 6: 4). IL-6 has been suggested as a central mediator of CRS toxicity (Tanaka et al. (2016) Immunotherapy. 8(8): 959-70) further supported by the effectiveness of Tocilizumab in treating severe CRS patients by blocking IL-6 signaling (Grupp et al. (2013) N. Engl. J. Med. 368: 1509-1518). Alternatively, IL1-beta signalling may be targeted by Anakinra (IL1-beta) to mitigate CRS. Typically, the agent will be used to target a single cytokine and prevent downstream signalling, and the initial secretion of IL-6 or IL1-beta is not prevented by the current treatments. In addition, existing treatments for CRS are not always effective and/or can have undesirable side effects. There is therefore a need for further therapies for the treatment or prevention of CRS.

### SUMMARY

The present invention relates to methods of treating or preventing a cytokine-related adverse event or disease such as cytokine release syndrome (CRS) in a subject using a cytokine inhibitor (e.g. IL-6 inhibitor), preferably wherein the cytokine inhibitor (e.g. IL-6 inhibitor) is pomalidomide, iberdomide, lenalidomide, avadomide or Compound 1 (4-(4-(4-(((2-(2,6-dioxopiperidin-3-yl)-1-oxoisoindolin-4-yl)oxy)methyl)benzyl)piperazin-1-yl)-3 -fluorobenzonitrile or an enantiomer, a mixture of enantiomers, a tautomer, an isotopolog or a pharmaceutically acceptable salt thereof).

In one aspect, the present invention provides a cytokine inhibitor (e.g. IL-6 inhibitor) for use in a method of treating or preventing a cytokine-related adverse event or disease such as cytokine release syndrome (CRS) in a subject, wherein the cytokine inhibitor (e.g. IL-6 inhibitor) is pomalidomide, iberdomide, lenalidomide, avadomide or Compound 1, and wherein Compound 1 is 4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, an isotopolog or a pharmaceutically acceptable salt thereof.

In another aspect, the present invention provides a method for treating or preventing a cytokine-related adverse event or disease such as cytokine release syndrome (CRS) in a subject, the method comprising administering to the subject a therapeutically effective dose of a cytokine inhibitor (e.g. IL-6 inhibitor), wherein the cytokine inhibitor (e.g. IL-6 inhibitor) is pomalidomide, iberdomide, lenalidomide, avadomide or Compound 1, wherein the therapeutically effective dose is a dose sufficient to reduce or prevent the development of CRS in the subject, and wherein Compound 1 is 4-(4-(4-(((2-(2,6-dioxopiperidin-3-yl)-1-oxoisoindolin-4-yl)oxy)methyl)benzyl)piperazin-l-yl)-3-fluorobenzonitrile, or an enantiomer, a mixture of enantiomers, a tautomer, an isotopolog or a pharmaceutically acceptable salt thereof.

In preferred embodiments, the cytokine-related adverse event or disease is cytokine release syndrome (CRS). In preferred embodiments, the subject has received or will receive a therapeutic agent that has caused or is likely to cause CRS. In particularly preferred embodiments, the therapeutic agent that has caused or is likely to cause CRS is a T cell engager.

In alternative embodiments, the cytokine-related adverse event or disease is Coronavirus disease 19 (COVID-19).

In yet further alternative embodiments, the cytokine-related adverse event or disease is cytokine-mediated neurotoxicity. In preferred embodiments, the subject has received or will receive a therapeutic agent that has caused or is likely to cause cytokine-mediated neurotoxicity. In particularly preferred embodiments, the therapeutic agent that has caused or is likely to cause cytokine-mediated neurotoxicity is a T cell engager.

In another aspect, the present invention provides a BCMA therapeutic agent for use in a method of treating a disorder associated with BCMA expression in a subject, wherein the method comprises:
a) administering to the subject the BCMA therapeutic agent, wherein the administering is likely to cause or has caused CRS in the subject; and
b) administering to the subject a cytokine inhibitor (e.g. IL-6 inhibitor) at a dose sufficient to prevent or reduce the development of CRS in the subject, wherein the cytokine inhibitor (e.g. IL-6 inhibitor) is pomalidomide, iberdomide, lenalidomide, avadomide or Compound 1, and wherein Compound 1 is 4-(4-(4-(((2-(2,6-dioxopiperidin-3-yl)-1-oxoisoindolin-4-yl)oxy)methyl)benzyl)piperazin-l-yl)-3-fluorobenzonitrile, or an enantiomer, a mixture of enantiomers, a tautomer, an isotopolog or a pharmaceutically acceptable salt thereof.

In yet another aspect, the present invention provides a method for treating a disorder associated with BCMA expression in a subject, wherein the method comprises:
a) administering to the subject a BCMA therapeutic agent, and
b) administering to the subject a cytokine inhibitor (e.g. IL-6 inhibitor), wherein the cytokine inhibitor (e.g. IL-6 inhibitor) is pomalidomide, iberdomide, lenalidomide, avadomide or Compound 1, and wherein Compound 1 is 4-(4-(4-(((2-(2,6-dioxopiperidin-3-yl)-1-oxoisoindolin-4-yl)oxy)methyl)benzyl)piperazin-l-yl)-3-fluorobenzonitrile, or an enantiomer, a mixture of enantiomers, a tautomer, an isotopolog or a pharmaceutically acceptable salt thereof.

In another aspect, the present invention provides a BCMA therapeutic agent for use in a method of treating a disorder associated with BCMA expression in a subject, wherein the method comprises:
a) administering to the subject a cytokine inhibitor (e.g. IL-6 inhibitor), wherein the cytokine inhibitor (e.g. IL-6 inhibitor) is pomalidomide, iberdomide, lenalidomide, avadomide or Compound 1, and wherein Compound 1 is 4-(4-(4-(((2-(2,6-dioxopiperidin-3-y1)-1-oxoisoindolin-4-yl)oxy)methyl)benzyl)piperazin-1-y1)-3-fluorobenzonitrile, or an enantiomer, a mixture of enantiomers, a tautomer, an isotopolog or a pharmaceutically acceptable salt thereof; and
b) following administration of the cytokine inhibitor, administering to the subject the BCMA therapeutic agent, wherein the administering is likely to cause CRS in the subject.

In a related aspect, the present invention provides a method for treating a disorder associated with BCMA expression in a subject, wherein the method comprises:
a) administering to the subject a cytokine inhibitor (e.g. IL-6 inhibitor), wherein the cytokine inhibitor (e.g. IL-6 inhibitor) is pomalidomide, iberdomide, lenalidomide, avadomide or Compound 1, and wherein Compound 1 is 4-(4-(4-(((2-(2,6-dioxopiperi din-3 -yl)-1-oxoisoindolin-4-yl)oxy)methyl)b enzyl)pip erazin-1-yl)-3 -fluorobenzonitrile, or an enantiomer, a mixture of enantiomers, a tautomer, an isotopolog or a pharmaceutically acceptable salt thereof; and
b) following administration of the cytokine inhibitor, administering to the subject a BCMA therapeutic agent, wherein the administering is likely to cause CRS in the subject.

In some embodiments, the cytokine inhibitor (e.g. IL-6 inhibitor) is administered as a first dose at least 1 day before the BCMA therapeutic agent, preferably at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days before the BCMA therapeutic agent.

In another aspect, the present invention provides a BCMA therapeutic agent for use in a method of treating a disorder associated with BCMA expression in a subject, wherein the method comprises:
a) administering to the subject the BCMA therapeutic agent, wherein the administering is likely to cause or has caused CRS in the subject; and
b) following administration of the BCMA therapeutic agent, administering to the subject a cytokine inhibitor (e.g. IL-6 inhibitor), wherein the cytokine inhibitor (e.g. IL-6 inhibitor) is pomalidomide, iberdomide, lenalidomide, avadomide or Compound 1, and wherein Compound 1 is 4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, an isotopolog or a pharmaceutically acceptable salt thereof.

In a related aspect, the present invention provides a method for treating a disorder associated with BCMA expression in a subject, wherein the method comprises:
a) administering to the subject a BCMA therapeutic agent, wherein the administering is likely to cause or has caused CRS in the subject; and
b) following administration of the BCMA therapeutic agent, administering to the subject a cytokine inhibitor (e.g. IL-6 inhibitor), wherein the cytokine inhibitor (e.g. IL-6 inhibitor) is pomalidomide, iberdomide, lenalidomide, avadomide or Compound 1, and wherein Compound 1 is 4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, an isotopolog or a pharmaceutically acceptable salt thereof.

In some embodiments, the BCMA therapeutic agent is administered as a first dose at least 1 day before the cytokine inhibitor, preferably at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days before the cytokine inhibitor.

In a further aspect, the present invention provides a BCMA therapeutic agent (e.g. a multispecific antibody which specifically binds to BCMA and to an antigen that promotes activation of one or more T cells) for use in a method of treating a disorder associated with BCMA expression in a subject, wherein the method comprises:
a) administering to the subject the BCMA therapeutic agent, wherein the administering is likely to cause or has caused CRS in the subject; and
b) administering to the subject Compound 1, wherein Compound 1 is 4-(4-(4-(((2-(2,6-dioxopiperidin-3-yl)-1-oxoisoindolin-4-yl)oxy)methyl)benzyl)piperazin-l-yl)-3-fluorobenzonitrile, or an enantiomer, a mixture of enantiomers, a tautomer, an isotopolog or a pharmaceutically acceptable salt thereof.

In certain embodiments of any aspect of the invention, the “subject” or “patient” is a human.

In particularly preferred embodiments, the BCMA therapeutic agent is a T cell engager.

In embodiments of any aspect of the invention, the T cell engager is a multispecific antibody that specifically binds to a target antigen (e.g. cancer antigen such as BCMA) and to an antigen that promotes activation of one or more T cells. In some embodiments, the antigen that promotes activation of one or more T cells is selected from the group consisting of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In preferred embodiments, the antigen that promotes activation of one or more T cells is CD3.

In alternative embodiments of any aspect of the invention, the T cell engager is a chimeric antigen receptor (CAR) directed to a target antigen (e.g. cancer antigen such as BCMA), or a T cell expressing at least one CAR directed to a target antigen (e.g. cancer antigen such as BCMA).

In embodiments of any aspect of the invention, the therapeutic agent that has caused or is likely to cause CRS or the BCMA therapeutic agent comprises an anti-BCMA antibody or antigen-binding fragment thereof comprising a CDR1H, CDR2H, CDR3H, CDR1L, CDR2L, and CDR3L region combination selected from:
a) CDR1H region of SEQ ID NO:21, CDR2H region of SEQ ID NO:22, CDR3H region of SEQ ID NO: 17, CDR1L region of SEQ ID NO:23, CDR2L region of SEQ ID NO:24, and CDR3L region of SEQ ID NO:20;
b) CDR1H region of SEQ ID NO:21, CDR2H region of SEQ ID NO:22, CDR3H region of SEQ ID NO: 17, CDR1L region of SEQ ID NO:25, CDR2L region of SEQ ID NO:26, and CDR3L region of SEQ ID NO:20;
c) CDR1H region of SEQ ID NO:21, CDR2H region of SEQ ID NO:22, CDR3H region of SEQ ID NO: 17, CDR1L region of SEQ ID NO:27, CDR2L region of SEQ ID NO:28, and CDR3L region of SEQ ID NO:20;
d) CDR1H region of SEQ ID NO:29, CDR2H region of SEQ ID NO:30, CDR3H region of SEQ ID NO: 17, CDR1L region of SEQ ID NO:31, CDR2L region of SEQ ID NO:32, and CDR3L region of SEQ ID NO:33;
e) CDR1H region of SEQ ID NO:34, CDR2H region of SEQ ID NO:35, CDR3H region of SEQ ID NO: 17, CDR1L region of SEQ ID NO:31, CDR2L region of SEQ ID NO:32, and CDR3L region of SEQ ID NO:33;
f) CDR1H region of SEQ ID NO:36, CDR2H region of SEQ ID NO:37, CDR3H region of SEQ ID NO: 17, CDR1L region of SEQ ID NO:31, CDR2L region of SEQ ID NO:32, and CDR3L region of SEQ ID NO:33; or
g) CDR1H region of SEQ ID NO: 15, CDR2H region of SEQ ID NO: 16, CDR3H region of SEQ ID NO: 17, CDR1L region of SEQ ID NO: 18, CDR2L region of SEQ ID NO: 19, and CDR3L region of SEQ ID NO:20.

In some embodiments of any aspect of the invention, the therapeutic agent that has caused or is likely to cause CRS or the BCMA therapeutic agent comprises an anti-BCMA antibody or antigen-binding fragment thereof comprising a VH and a VL selected from the group consisting of:
a) a VH region of SEQ ID NO: 10 and a VL region of SEQ ID NO: 12;
b) a VH region of SEQ ID NO: 10 and a VL region of SEQ ID NO: 13;
c) a VH region of SEQ ID NO: 10 and a VL region of SEQ ID NO: 14;
d) a VH region of SEQ ID NO:38 and a VL region of SEQ ID NO: 12;
e) a VH region of SEQ ID NO:39 and a VL region of SEQ ID NO: 12;
f) a VH region of SEQ ID NO: 40 and a VL region of SEQ ID NO: 12; or
g) a VH region of SEQ ID NO: 9 and a VL region of SEQ ID NO: 11.

In preferred embodiments of any aspect of the invention, the therapeutic agent that has caused or is likely to cause CRS or the BCMA therapeutic agent comprises an anti-BCMA antibody or antigen-binding fragment thereof comprising a VH region of SEQ ID NO: 10 and a VL region of SEQ ID NO: 14.

In alternative embodiments of any aspect of the invention, the therapeutic agent that has caused or is likely to cause CRS or the BCMA therapeutic agent comprises an anti-BCMA antibody antigen-binding fragment thereof comprises a VH comprising a CDR1H of SEQ ID NO:64, a CDR2H of SEQ ID NO: 65 and a CDR3H of SEQ ID NO: 66, and a VL comprising a CDR1L, a CDR2L and a CDR3L set of sequences selected from:
a) CDR1L of SEQ ID NO:67, CDR2L of SEQ ID NO:68, and CDR3L of SEQ ID NO:69, optionally wherein the BCMA therapeutic agent comprises a VH of SEQ ID NO: 76 and a VL of SEQ ID NO: 77;
b) CDR1L of SEQ ID NO:70, CDR2L of SEQ ID NO:71, and CDR3L of SEQ ID NO:72, optionally wherein the BCMA therapeutic agent comprises a VH of SEQ ID NO:76 and a VL of SEQ ID NO: 78; or
c) CDR1L of SEQ ID NO:73, CDR2L of SEQ ID NO:74, and CDR3L of SEQ ID NO:75 optionally wherein the BCMA therapeutic agent comprises a VH of SEQ ID NO: 76 and a VL of SEQ ID NO:79.

In some embodiments, the multispecific antibody comprises an anti-CD3 antibody, or antigen binding fragment thereof. In some embodiments, the anti-CD3 antibody, or antigen binding fragment thereof comprises a variable domain VH comprising the heavy chain CDRs of SEQ ID NOs: 1, 2 and 3 as respectively heavy chain CDR1H, CDR2H and CDR3H and a variable domain VL comprising the light chain CDRs of SEQ ID NOs: 4, 5 and 6 as respectively light chain CDR1L, CDR2L and CDR3L. In preferred embodiments, the anti-CD3 antibody, or antigen binding fragment thereof, comprises a VH region of SEQ ID NO:7 and a VL region of SEQ ID NO: 8.

In particularly preferred embodiments, the multispecific antibody comprises an anti-BCMA antibody, or antigen binding fragment thereof, comprising a VH region of SEQ ID NO: 10 and a VL region of SEQ ID NO: 14, and an anti-CD3 antibody, or antigen binding fragment thereof, comprising a VH region of SEQ ID NO:7 and a VL region of SEQ ID NO:8.

In some embodiments, the multispecific antibody is a bispecific antibody. In some embodiments, the bispecific antibody is bivalent (e.g. 1+1 format). In alternative embodiments, the bispecific antibody is trivalent (e.g. 2+1 format). In some embodiments, the trivalent bispecific antibody has the format: CD3 Fab - BCMA Fab - BCMA Fab; or BCMA Fab – CD3 Fab – BCMA Fab (i.e. when no Fc is present). Alternatively, the trivalent bispecific antibody may have the format: BCMA Fab – Fc – CD3 Fab – BCMA Fab; BCMA Fab – Fc – BCMA Fab – CD3 Fab; or CD3 Fab – Fc – BCMA Fab – BCMA Fab (i.e. when an Fc is present). In preferred embodiments, the trivalent bispecific antibody has the format BCMA Fab – Fc – CD3 Fab – BCMA Fab.

In some embodiments, the anti-CD3 Fab comprises a light chain and a heavy chain, wherein the light chain is a crossover light chain that comprises a variable domain VH and a constant domain CL, and wherein the heavy chain is a crossover heavy chain that comprises a variable domain VL and a constant domain CH1.

In some embodiments, the CH1 domain of the anti-BCMA Fab fragment comprises the amino acid modifications K147E/D and K213E/D (numbered according to EU numbering) and a corresponding immunoglobulin light chain comprising a CL domain having amino acid modifications E123K/R/H and Q124K/R/H (numbered according to Kabat).

In some embodiments, the multispecific (e.g. bispecific) antibody further comprises an Fc. In some embodiments, the Fc is an IgGl Fc. In some embodiments, the (e.g. IgGl) Fc comprises a first Fc chain comprising first constant domains CH2 and CH3, and a second Fc chain comprising second constant domains CH2 and CH3, and wherein:
a) the first CH3 domain comprises the modifications T366S, L368A and Y407V, or conservative substitutions thereof (numbered according to EU numbering); and
b) the second CH3 domain comprises the modification T366W, or conservative substitutions thereof (numbered according to EU numbering).

In some embodiments, the (e.g. IgGl) Fc comprises:
a) the modifications L234A, L235A and P329G (numbered according to EU numbering); and/or
b) the modifications D356E, and L358M (numbered according to EU numbering).

In further embodiments, the bispecific antibody according to the invention comprises a heavy and light chain set of the polypeptides set forth in the following SEQ ID NOs:
83A10-TCBcv: 48, 45, 46, 47 (x2);
22-TCBcv: 48, 52, 53, 54 (x2); or
42-TCBcv: 48, 55, 56, 57 (x2).

In a preferred embodiment, the bispecific antibody according to the invention is 42-TCBcv and comprises a heavy and light chain set of the polypeptides set forth in SEQ ID NO:48, SEQ ID NO:55, SEQ ID NO:56, and two copies of SEQ ID NO:57.

In embodiments of any aspect of the invention, the BCMA therapeutic agent is AMG-420 [Amgen], BCMA tri-specific [Affirmed], AFM26 [Affirmed], Ab-957 [Janssen], BCMA/PD-L1 [Immune pharmaceuticals], AMG-701 [Amgen], PF-06863135 [Pfizer], REGN-5458 [Regeneron / Sanofi], or TNB-383B [TeneoBio].

In embodiments of any aspect of the invention, the BCMA therapeutic agent is a chimeric antigen receptor (CAR) directed to BCMA, or a T cell expressing at least one CAR directed to BCMA (“BCMA CAR T cell”).

In some embodiments, the BCMA CAR T cell is idecabtagene-vicleucel (ide-cel), bb21217, JCARH125 (orva-cel), KITE-585 (Kite Pharmaceuticals), P-BCMA-101 (Poseida Therapeutics), CART-BCMA (Novartis), LCAR-B38M (Legend Biotech), JNJ-528 (Janssen Biotech), P-BCMA-101 (Poseida Therapeutics), CT053 (CARsgen Therapeutics), CTX120 (CRISPR Therapeutics), ET140 (Juno Therapeutics), UCART-BCMA (Cellectis), P-BCMA-101 (Poseida), JNJ-528ALCAR-B38M (Johnson & Johnson). In certain embodiments, the BCMA CAR T cell is MCARH171, FCARH143, CTX120, CT053 (First Affiliated Hospital of Wenzhou Medical University, CN), or BCMA-CART (Hrain Biotechnology, Shanghai CN). In preferred embodiments, the BCMA CAR T cell is idecabtagene-vicleucel, bb21217, or JCARH125.

In embodiments of any aspect of the invention, the therapeutic agent that has caused or is likely to cause CRS or the BCMA therapeutic agentis an antibody-drug conjugate (ADC).

In embodiments of any aspect of the invention, the cytokine inhibitor (e.g. IL-6 inhibitor) is administered before (e.g. within 12 or 24 hours before) administration of the therapeutic agent (e.g. BCMA therapeutic agent), on the same day as administration of the therapeutic agent (e.g. BCMA therapeutic agent) or after (e.g. within 12 or 24 hours after) administration of the therapeutic agent (e.g. BCMA therapeutic agent).

In some embodiments of any aspect of the invention, the cytokine inhibitor (e.g. IL-6 inhibitor) is administered as a pre-treatment before administration of the therapeutic agent (e.g. BCMA therapeutic agent). In some embodiments, the cytokine inhibitor (e.g. IL-6 inhibitor) is administered as one or more doses before (e.g. 1, 2, 3, 4, 5, 6, 7, 8, 9, 10, 11, 12, 13, 14, 15, 16, 17, 18, 19, 20, 21, 22, 23, 24, 25, 26, 27 or 28 days before) administration of the therapeutic agent (e.g. BCMA therapeutic agent). In some embodiments, the cytokine inhibitor (e.g. IL-6 inhibitor) is administered on days 1, 2, 3, 4, 5, 6 and 7, before administration of the therapeutic agent (e.g. BCMA therapeutic agent) on day 8.

In alternative embodiments, the therapeutic agent (e.g. BCMA therapeutic agent) is administered before administration of the cytokine inhibitor (e.g. IL-6 inhibitor). In some embodiments, the therapeutic agent (e.g. BCMA therapeutic agent) is administered as one or more doses before (e.g. 1, 2, 3, 4, 5, 6, 7, 8, 9, 10, 11, 12, 13, 14, 15, 16, 17, 18, 19, 20, 21, 22, 23, 24, 25, 26, 27 or 28 days before) administration of the cytokine inhibitor (e.g. IL-6 inhibitor). In some embodiments, the therapeutic agent (e.g. BCMA therapeutic agent) is administered on day 1 and optionally day 4, before administration of the cytokine inhibitor (e.g. IL-6 inhibitor) on day 8.

In some embodiments of any aspect of the invention, the cytokine inhibitor (e.g. IL-6 inhibitor) is administered within 12 hours after diagnosis of CRS.

Aspects and embodiments of the invention are set out in the appended claims. These and other aspects and embodiments of the invention are also described herein.

### BRIEF DESCRIPTION OF FIGURES

The present invention will now be described in more detail with reference to the attached Figures, in which:

FIG. 1 illustrates different formats of bispecific bivalent antibodies for use in the present invention, which comprise Fab fragments binding to a T cell antigen (CD3 is illustrated) and BCMA in the format Fab BCMA– Fc – Fab CD3. The CD3 Fab may include a VH-VL crossover to reduce light chain mispairing and side-products. Amino acid substitutions “RK/EE” may be introduced in CL-CH1 to reduce light chain mispairing/side products in production. The CD3 Fab and BCMA Fab may be linked to each other with flexible linkers.

FIG. 2 illustrates different formats of bispecific trivalent antibodies for use in the present invention, which comprise Fab fragments binding to a T cell antigen (CD3 is illustrated) and BCMA in the following formats: Fab BCMA – Fc – Fab CD3 – Fab BCMA (A,B); Fab BCMA –Fc – Fab BCMA – Fab CD3 (C,D). The CD3 Fab may include a VH-VL crossover to reduce light chain mispairing and side-products. Amino acid substitutions “RK/EE” may be introduced in CL-CH1 to reduce light chain mispairing/side products in production. The CD3 Fab and BCMA Fab may be linked to each other with flexible linkers.

FIG. 3 illustrates further formats of bispecific bivalent antibodies for use in the present invention, which comprise Fab fragments binding to a T cell antigen (CD3 is illustrated) and BCMA in the following formats: Fc – Fab CD3 – Fab BCMA (A,B); Fc – Fab BCMA – Fab CD3 (C,D). The CD3 Fab may include a VH-VL crossover to reduce light chain mispairing and side-products. Amino acid substitutions “RK/EE” may be introduced in CL-CH1 to reduce light chain mispairing/side products in production. The CD3 Fab and BCMA Fab may be linked to each other with flexible linkers

FIG. 4 shows that LPS-induced IL-6 secretion from monocytes is diminished by pre-treatment with certain IMiD/CELMoD agents. Monocytes from healthy volunteers were seeded at a concentration of 1×10⁶ cells/ml and treated with the indicated concentration of IMiD/CELMoD agents overnight (~17 hours). The next morning, the monocytes were incubated with the indicated concentration of LPS for 4 hours, before IL-6 levels in the supernatant were assessed using an MSD assay.

FIG. 5 shows that LPS-induced IL-6 secretion from monocytes is diminished by pre-treatment with 1-100 nM Compound 1. Monocytes from healthy volunteers were seeded at a concentration of 1×10⁶ cells/ml and treated with the indicated concentration of Compound 1 overnight. The next morning, the monocytes were incubated with the indicated concentration of LPS for 4 hours, and then stimulation with 5 ug/ml of nigericin for 1 hour, before IL-6 levels in the supernatant were assessed using an MSD assay.

FIG. 6 shows that LPS-induced IL-6 secretion from macrophages is diminished by pre-treatment with certain IMiD/CELMoD agents. Monocytes from healthy volunteers were seeded at a concentration of 3×10⁵ cells/ml for 4 days with RPMI 1640 media containing M-CSF (50 ng/mL) (50% media replaced after 2 days) to obtain naive macrophages (M0). At the end of day 4, macrophages were treated with the indicated concentration of IMiD/CELMoD agents overnight (~17 hours). The next morning, the macrophages were stimulated with the indicated concentration of LPS for 8 hours, before IL-6 levels in the supernatant were assessed using an MSD assay.

FIG. 7 shows that LPS-induced secretion of proinflammatory cytokines is inhibited by pre-treatment with thalidomide derivatives (lenalidomide and pomalidomide are illustrated in FIGS. 7B and 7C, respectively). Peripheral blood mononuclear cells (PBMCs) from healthy volunteers were seeded at a concentration of 2×10⁶ cells/ml and treated with the indicated concentrations of IMiD compounds or control DMSO (0.25%) for 1 hour, before stimulation with 1 ng/ml LPS. After 18 hours, proinflammatory cytokine levels in the supernatant were assessed by multiplex cytokine analysis.

FIG. 8 shows that LPS-induced secretion of IL-6, TNF-alpha and IL1-beta from PBMCs is simultaneously suppressed by pre-treatment with IMiD/CELMoD agents. Fresh PBMCs isolated from healthy volunteers were seeded at a concentration of 1×10⁶ cells/ml and treated with the indicated concentration of IMiD drugs/CELMoD agents or DMSO overnight before stimulation with the indicated concentration of LPS. After 24 hours, the cell culture media was collected and IL-6, TNF-alpha and IL1-beta levels in the supernatant were assessed using an MSD assay. ULOQ = Upper limit of quantification.

FIG. 9 shows that CC-93269-induced secretion of IL-6 from PBMCs with BCMA-expressing target cells is suppressed by pre-treatment with IMiD/CELMoD agents. Fresh PBMCs were isolated from healthy volunteers, the percentage of CD3+ T-cells (total) was quantified by flow cytometry, and the PBMCs were treated with 1 nM Compound 1 (CC-92480) or control DMSO overnight. The next morning, target cells (K562-BCMA, which overexpress surface BCMA at a very high level) or control cells (K562-MCB, no BCMA expression) were added to the wells at a ratio of 5:1, T-cells to target cells, followed by addition of CC-93269 at the indicated concentrations. At 6-, 24- and 48-hours post-incubation, the cell culture media was collected and levels of IL-6 in the supernatant were assessed using an MSD assay.

FIG. 10 shows that Compound 1 (CC-92480) suppresses CC-93269-induced IL-6 secretion from a co-culture of PBMCs + K562-BCMA cells at 24 hours, for CC-93269 concentrations up to 10,000 ng/mL (FIG. 10A) and independently across 15 healthy donors (FIG. 10B). FIG. 10B shows the IL-6 levels for the Compound 1 (CC-92480)-pre-treated samples normalized relative to the control DMSO pre-treated samples, with the control set at 100%. The circles indicate independent donors and the bars indicate median values; not all donors are shown because a data cut-off for the bottom ~10% of IL-6 concentrations, below 200 pg/ml, was used to eliminate small changes that could skew the result and to increase confidence in results.

FIG. 11 shows that CC-93269-induced secretion of IL-6 from PBMCs with BCMA-expressing target cells is suppressed by pre-treatment with 1000 nM lenalidomide, 100 nM pomalidomide, 10 nM iberdomide or 1 nM Compound 1 (CC-92480). Fresh PBMCs were isolated from healthy volunteers, the percentage of CD3+ T-cells (total) was quantified by flow cytometry, and the PBMCs were treated with the indicated concentrations of IMiD/CELMoD agents or control DMSO overnight. The next morning, target cells (K562-BCMA, which overexpress surface BCMA at a very high level) were added to the wells at a ratio of 5:1, T-cells to target cells, followed by addition of CC-93269 at the indicated concentrations. At 24 hours post-incubation, the cell culture media was collected and levels of IL-6 in the supernatant were assessed using an MSD assay.

FIG. 12 shows that pre-treatment with Compound 1 (CC-92480) potentiates CC-93269-induced TNF-alpha and IL-2 secretion from PBMCs with target K562-BCMA cells after 24 hours. Co-cultures of PBMCs, target K562-BCMA cells and CC-93269 were prepared as for FIG. 9. At 6-, 24- and 48-hours post-incubation, the cell culture media was collected and levels of TNF-alpha (FIG. 12A) and IL-2 (FIG. 12B) in the supernatant were assessed using an MSD assay. ULOQ = Upper limit of quantification.

FIG. 13 shows that CC-93269-induced IL1-beta secretion from PBMCs with K562 target cells is suppressed by pre-treatment with Compound 1 (CC-92480). Co-cultures of PBMCs, target K562-BCMA cells and CC-93269 were prepared as for FIG. 9. At 6-, 24- and 48-hours post-incubation, the cell culture media was collected and levels of IL1-beta in the supernatant were assessed using an MSD assay (FIG. 13A). FIG. 13B shows the IL1-beta levels at 24 hours for the Compound 1 (CC-92480) samples normalized relative to the control DMSO samples, for 15 independent healthy donors. The circles indicate independent donors and the bars indicate median values; not all donors are shown because a data cut-off for the bottom ~10% of IL1-beta concentrations, below 100 pg/ml, was used to eliminate small changes that could skew the result and to increase confidence in results.

FIG. 14 shows that CC-93269-induced secretion of IL-6 from PBMCs with H929 target cells (H929 is a multiple myeloma cell line) is suppressed by pre-treatment with IMiD/CELMoD agents. Fresh PBMCs were isolated from healthy volunteers, the percentage of CD3+ T-cells (total) was quantified by flow cytometry, and the PBMCs were treated with 1 nM Compound 1 (CC-92480) or control DMSO overnight. The next morning, target H929 cells were added to the wells at a ratio of 5:1, T-cells to target cells, followed by addition of CC-93269 at indicated concentrations. At 6-, 24- and 48-hours post-incubation, the cell culture was collected and levels of IL-6 in the supernatant were assessed using an MSD assay. Data from two independent healthy donors is shown in FIGS. 14A and 14B.

FIG. 15 shows the effects of prior exposure to Cereblon Modulating (CM) agents during induction of T-cell exhaustion on CC-93269 induced cytolytic activity. Cell growth kinetics of A) NCI-H929 and B) OPM-2 cells in CC-93269 (47 pM) cytotoxicity assays on the IncuCyte® S3 Live-Cell Analysis System. Healthy donor T-cells were used as effector cells with or without chronic stimulation with anti-CD3/CD28 (7 days) in the presence of DMSO or CM agents (100 nM Pomalidomide, 10 nM CC-220 or 1 nM CC-92480). T-cells and NCI-H929 cells were co-cultured at E:T ratios of 1:4 (NCI-H929) or 1:2 (OPM-2). In assays in which CD3+ T-cells underwent chronic anti-CD3/CD28 stimulation for 7 days, functional T-cell exhaustion (i.e. loss of cytolytic function) was observed in DMSO-treated cells when compared to freshly thawed T-cells. In contrast, prior exposure to CM agents prevented functional TBMS cell exhaustion and CC-93269 activity was similar or better to those observed with freshly thawed T-cells. Values shown represent the mean ± standard deviation of AUC values of 2-3 replicates from the same experiment. AUC = Area under the curve; DMSO = Dimethyl sulfoxide; POM = Pomalidomide; CC-220 = iberdomide. * p < 0.05, ** p < 0.01 versus freshly thawed T-cells, or as indicated, by analysis of variance (ANOVA).

### DETAILED DESCRIPTION

As used herein, the articles “a” and “an” may refer to one or to more than one (e.g. to at least one) of the grammatical object of the article.

About” may generally mean an acceptable degree of error for the quantity measured given the nature or precision of the measurements. Exemplary degrees of error are within 20 percent (%), typically, within 10%, and more typically, within 5% of a given value or range of values.

Embodiments described herein as “comprising” one or more features may also be considered as disclosure of the corresponding embodiments “consisting of′ and/or “consisting essentially of′ such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### Cytokine Inhibitor

The cytokine inhibitor (e.g. IL-6 inhibitor) of the invention may be a Cereblon E3 ligase modulator (CELMoD) or an immunomodulatory imide drug (IMiD) that modulates Cereblon. Cereblon interacts with damaged DNA binding protein 1 and forms an E3 ubiquitin ligase complex with Cullin 4 and the E2-binding protein ROC1 (known as RBX1) where it functions as a substrate receptor to select proteins for ubiquitination.

In preferred embodiments, the cytokine inhibitor (e.g. IL-6 inhibitor) is a thalidomide derivative, Compound 1, or a combination thereof.

“Thalidomide derivative” as used herein relates to 2-(2,6-dioxopiperidin-3-yl)-2,3 dihydro-1H-isoindole-1,3-dione and immunotherapeutic derivatives thereof such as, but not limited to: lenalidomide (3-(4-amino-1-oxo-2,3-dihydro-1H-isoindol-2-yl) piperidine-2,6-dione; CAS Registry Number 191732-72-6); pomalidomide (4-amino-2-(2,6-dioxopiperidine-3-yl)isoindoline-1,3-dione; CAS Registry Number 19171-19-8); iberdomide ((S)-3-(4-((4-(morpholinomethyl)benzyl)oxy)-1-oxoisoindolin-2-yl)piperidine-2,6-dione; CAS Registry Number 1323403-33-3); avadomide (3-(5-amino-2-methyl-4-oxo-3,4-dihydroquinazolin-3-yl)piperidine-2,6-dione; CAS Registry Number 1398053-45-6), and the respective salts (preferably HCl salts 1: 1); or the compounds referred to in Example 4 as Compounds A and B. Thalidomide derivatives are IMiD agents that modulate Cereblon. In particularly preferred embodiments, the thalidomide derivative is pomalidomide, lenalidomide, iberdomide, avadomide, or a combination thereof.

“Compound 1” as used herein relates to 4-(4-(4-(((2-(2,6-dioxopiperidin-3-yl)-1-oxoisoindolin-4-yl)oxy)methyl)benzyl)piperazin-l-yl)-3-fluorobenzonitrile (CAS Registry Number 2259648-80-9) or an enantiomer, a mixture of enantiomers, a tautomer, an isotopolog, or a pharmaceutically acceptable salt thereof. Compound 1 is a CELMoD agent, also referred to herein as CC-92480. The structure of Compound 1 is as follows:

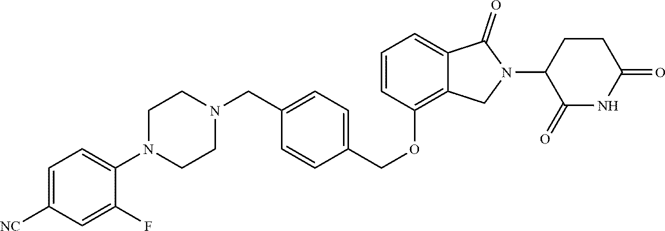

As used herein, an “isotopolog” refers to a compound containing at least one atom having an isotopic composition other than the natural isotopic composition of that atom. The term “isotopic composition” refers to the amount of each isotope present for a given atom. In some embodiments, there are provided isotopologs of Compound 1, for example, deuterium, carbon-13, or nitrogen-15 enriched compounds.

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzyl phenethylamine, l-para-chlorobenzyl-2-pyrrolidin-l′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and di sodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, fumarates and organic sulfonates.

The cytokine inhibitors (e.g. IL-6 inhibitors) of the invention are commercially available and/or can be prepared by methods known to one of skill in the art. Methods of preparing iberdomide are described e.g. in US 20110196150; and methods of preparing Compound 1 are described e.g. in WO-A1-2019226761.

In preferred embodiments of any aspect of the invention, the cytokine inhibitor is a proinflammatory cytokine inhibitor (e.g. an IL-6 inhibitor or an IL1-beta inhibitor).

### Therapeutic Applications

The invention is based, at least in part, on the treatment or prevention of a cytokine-related adverse event or disease in a patient using a cytokine inhibitor (e.g. IL-6 inhibitor) such as lenalidomide, pomalidomide, iberdomide, avadomide, Compound 1, or any combination thereof.

As used herein, the terms “treat”, “treating” or “treatment” and the like refer to obtaining a desired pharmacologic and/or physiologic effect. Preferably, the effect is therapeutic, i.e., the effect partially or completely cures a disease and/or adverse symptom. Accordingly, the pharmacologic and/or physiologic effect may reduce the severity of a disease and/or adverse symptom. Alternatively, the pharmacologic and/or physiologic effect may be prophylactic, i.e., the effect completely or partially prevents a disease or adverse symptom.

As used herein, the terms “prevent”, “preventing” or “prevention” and the like refer to suppressing and/or delaying the onset, development and/or worsening of the disease and/or adverse symptoms.

The present inventors have unexpectedly found that certain IMiD and CELMoD agents can inhibit the secretion of proinflammatory cytokines, particularly IL-6 and IL1-beta, from macrophages and/or monocytes (see Examples 1 to 5), which can be artificially induced by lipopolysaccharides, LPS (Rossol et al. (2011) Crit. Rev. Immunol. 31(5): 379-446). Based on this finding, the inventors have recognized that IMiD and CELMoD agents can be used in adverse events in which elevated levels of cytokines, in particular elevated levels of IL-6 and IL1-beta, is thought to play a significant role, such as CRS, cytokine-mediated neurotoxicity or Coronavirus disease 2019 (COVID-19).

In preferred embodiments, the cytokine inhibitors (e.g. IL-6 inhibitors) of the invention treat or prevent a cytokine-related adverse event in a patient, such as CRS or cytokine-mediated neurotoxicity. Preferably, the cytokine-related adverse event or disease is not a malignant disease. In some embodiments the cytokine-related adverse event or disease is not a solid tumor, a metastatic cancer or a soft tissue tumor. For example, in some embodiments the cytokine-related adverse event or disease is not breast cancer.

CRS is thought to be triggered by a massive release of cytokines, mainly IFN-y but also TNF-α, by activated immune effector cells, e.g. T cells activated by a T cell-engaging immunotherapy. Without being bound by theory, these cytokines induce activation of other immune cells, including macrophages and monocytes, resulting in a rapid and/or large secretion of proinflammatory cytokines from these cells. The activated macrophages and monocytes secrete IL-6 and IL1-beta which, in a positive feedback loop manner, activate more T cells and other immune cells (Shimabukuro-Vornhagen A et al. (2018) J. Immunother. Cancer. 6(1): 56). This inflammatory cascade can result in a cytokine storm and systemic inflammatory response.

Elevated IL-6 and IL1-beta levels are thought to be a major mediator of toxicity in CRS. For example, a recent study identified high levels of IL-6 to be most strongly associated with severe CRS over the first month compared with other cytokines (Teachey DT et al. (2016) Cancer Discov. 6(6): 664-679.

In some embodiments of any aspect of the invention, the cytokine inhibitors (e.g. IL-6 inhibitors) of the invention reduce proinflammatory cytokine (e.g. IL-6) secretion from bone marrow stromal cells, osteoblasts, Kuppfer cells, peripheral blood mononuclear cells (PBMCs), T-cells, B-cells and/or myeloid cells (e.g. monocytes and/or macrophages). In some embodiments, the cytokine inhibitors (e.g. IL-6 inhibitors) of the invention reduce proinflammatory cytokine (e.g. IL-6) secretion from PBMCs. In preferred embodiments, the cytokine inhibitors (e.g. IL-6 inhibitors) of the invention reduce proinflammatory cytokine (e.g. IL-6) secretion from monocytes and/or macrophages.

The proinflammatory cytokine secretion may be mediated by a T cell engager disclosed herein (e.g. 42-TCBcv). In some embodiments, the cytokine inhibitors (e.g. IL-6 inhibitors) of the invention reduce T cell engager (e.g. 42-TCBcv) mediated proinflammatory cytokine (e.g. IL-6) secretion. In preferred embodiments, the T cell engager (e.g. 42-TCBcv) mediated proinflammatory cytokines include at least IL-6. In particularly preferred embodiments, the T cell engager (e.g. 42-TCBcv) mediated proinflammatory cytokines include at least IL-6 and IL1-beta. Thus, the cytokine inhibitors of the invention can provide the advantage of reducing multiple cytokines. In contrast, existing treatments such as Tocilizumab and Anakinra target a single cytokine.

T cell engager (e.g. 42-TCBcv) mediated proinflammatory cytokine (e.g. IL-6) secretion may be measured in a co-culture of PBMCs and BCMA-expressing target cells (e.g. multiple myeloma cells). In some embodiments, the cytokine inhibitors (e.g. IL-6 inhibitors) of the invention reduce T cell engager (e.g. 42-TCBcv) mediated IL-6 secretion from a co-culture of PBMCs and BCMA-expressing target cells (e.g. multiple myeloma cells) at a ratio of 5:1, T-cells to target cells, wherein the fold decrease is at least 1.5-fold, 2.0-fold, 2.5-fold, 3.0-fold, 3.5-fold, 4.0-fold, 4.5-fold, or 5.0-fold, compared with IL-6 secretion from a co-culture which is not treated with the cytokine inhibitor. Optionally, the fold decrease is up to 5.0-fold. Thus, in some embodiments, the fold decrease is between 1.5-fold and 5.0-fold.

In some embodiments, the cytokine inhibitors of the invention reduce T cell engager (e.g. 42-TCBcv) mediated IL1-beta secretion from a co-culture of PBMCs and BCMA-expressing target cells (e.g. multiple myeloma cells) at a ratio of 5:1, T-cells to target cells, by at least 2-fold, 4-fold, 6-fold, 8-fold or 10-fold, compared with IL1-beta secretion from a co-culture which is not treated with the cytokine inhibitor. Optionally, the fold decrease is up to 10-fold. Thus, in some embodiments, the fold decrease is between 2-fold and 10-fold.

In some embodiments, the cytokine inhibitors (e.g. IL-6 inhibitors) of the invention reduce T cell engager (e.g. 42-TCBcv) mediated proinflammatory cytokine (e.g. IL-6) secretion from a co-culture of PBMCs and BCMA-expressing target cells (e.g. multiple myeloma cells), at concentrations of 42-TCBcv up to about 100 mg/mL, optionally up to about 10,000 ng/mL, e.g. up to about 1000 ng/mL.

In some embodiments, the cytokine inhibitors (e.g. IL-6 inhibitors) of the invention do not reduce T cell engager (e.g. 42-TCBcv) mediated secretion of cytokines related to T-cell cytotoxicity. optionally wherein the cytokines related to T-cell cytotoxicity are one or more of TNF-alpha, IL-2 and/or IFN-gamma.

Alternatively, the proinflammatory cytokine secretion may be TLR4-mediated. Thus, the cytokine inhibitors (e.g. IL-6 inhibitors) of the invention may reduce TLR4-mediated proinflammatory cytokine (e.g. IL-6) secretion from bone marrow stromal cells, osteoblasts, Kuppfer cells, PBMCs, B-cells and/or myeloid cells (e.g. monocytes and/or macrophages). Preferably, the TLR4-mediated proinflammatory cytokine secretion is LPS-induced. In some embodiments, the cytokine inhibitors (e.g. IL-6 inhibitors) of the invention reduce TLR4-mediated proinflammatory cytokine (e.g. IL-6) secretion from PBMCs, monocytes and macrophages. In preferred embodiments, the TLR4-mediated proinflammatory cytokines include at least IL-6. In particularly preferred embodiments, the LPS-induced proinflammatory cytokines include at least IL-6, IL1-beta, and TNF-alpha.

In some embodiments, the proinflammatory cytokines are one or more of IL-6, IL-8, IL1-beta, GM-CSF, MDC, MIP-1 alpha, and TNF-alpha. In preferred embodiments, the proinflammatory cytokines include at least IL-6. In some embodiments, the proinflammatory cytokines exclude IL1-beta. In some embodiments, the proinflammatory cytokines include at least IL-6 and IL1-beta.

In preferred embodiments of any aspect of the invention, the cytokine inhibitors of the invention reduce IL-6 secretion from bone marrow stromal cells, osteoblasts, Kuppfer cells, PBMCs, B-cells and/or myeloid cells. In preferred embodiments, the cytokine inhibitors of the invention reduce IL-6 secretion from monocytes and/or macrophages. In particularly preferred embodiments of any aspect of the invention, the cytokine inhibitors of the invention reduce TLR4-mediated IL-6 secretion from bone marrow stromal cells, osteoblasts, Kuppfer cells, PBMCs, B-cells and/or myeloid cells, preferably monocytes and/or macrophages. In preferred embodiments, the cytokine inhibitors (e.g. IL-6 inhibitors) of the invention treat or prevent CRS. CRS is commonly treated according to a grade-adapted strategy using the ASTCT or CTCAE grading systems, the first of which was most recently defined by (Lee DW et al. (2019) Biol. Blood Marrow Transplant. 25(4): 625-638) and the second was most recently published by the National Cancer Institute in November 2017. The present invention may be used to treat or prevent any grade of CRS. In some embodiments, the CRS is minimum grade 1, minimum grade 2, minimum grade 3 or minimum grade 4. In preferred embodiments, the CRS is minimum grade 3 or 4. In some embodiments, the subject has received or will receive a therapeutic agent that has caused or is likely to cause CRS. In some embodiments, the treatment or prevention of CRS reduces the severity and/or grade of CRS.

In some embodiments, the therapeutic agent that has caused or is likely to cause CRS is selected from a T cell engager (e.g. a multispecific antibody, CAR or CAR T cell), a monoclonal antibody which specifically binds to a target antigen (e.g. a cancer antigen such as BCMA, CD19, CD20 or CD28), a monoclonal antibody which specifically binds to a T cell antigen (e.g. CD3), or an antibody drug conjugate. In preferred embodiments, the therapeutic agent is a T cell engager.

The present inventors have further found that the cytokine inhibitors (e.g. IL-6 inhibitors) of the invention can enhance the cell killing ability of T-cell engaging therapeutic agents (e.g. for the treatment of multiple myeloma that are likely to cause or has caused CRS) and/or may prevent T-cell exhaustion and thus preserve the T-cell mediated killing of T cell engagers (see Example 9).

Cytokine-mediated neurotoxicity is another adverse event of T cell-engaging immunotherapy. Vascular leakage and disruption of the blood-brain barrier, central to neurotoxicity, has been linked with elevated IL-6 (Khadka RH et al. (2019) Immunotherapy. 11(10): 851-857). An earlier peak of IL-6 serum concentration after CAR T-cell therapy is associated with a higher risk of grade ≥ 4 neurotoxicity (Gust J et al. (2017) Cancer Discov. 7(12): 1404-1419). Preclinical studies have confirmed myeloid cells including monocytes and macrophages are the primary source of IL-6 in T cell-engaging immunotherapies (Yu S et al. (2017) J. Hematol. Oncol. 10(1):155; Teachey DT et al. (2013) Blood. 121(26): 5154-5157). Neurotoxicity associated with CAR T-cell therapy and T cell-engaging immunotherapies may be referred to as “immune effector cell associated neurotoxicity syndrome (ICANS)” (Lee et al. (2018) Biol. Blood Marrow Transplant. 25(4): 625-638). Elevated cytokines, including IL-1, are known to be a part of the pathophysiology of ICANS.

In one aspect, the present invention provides a method for treating or preventing cytokine-mediated neurotoxicity in a subject, the method comprising administering to the subject a therapeutically effective dose of a cytokine inhibitor (e.g. IL-6 inhibitor), wherein the cytokine inhibitor (e.g. IL-6 inhibitor) is pomalidomide, iberdomide, lenalidomide, avadomide or Compound 1, wherein the therapeutically effective dose is a dose sufficient to reduce or prevent the development of cytokine-mediated neurotoxicity in the subject. In some embodiments, the cytokine-mediated neurotoxicity is immune effector cell associated neurotoxicity syndrome (ICANS).

In alternative embodiments, the cytokine inhibitors (e.g. IL-6 inhibitors) of the invention treat or prevent a cytokine-related disease in a patient, such as Coronavirus disease 2019 (COVID-19).

Coronavirus disease 2019 (COVID-19) is a potentially severe acute respiratory infection caused by severe acute respiratory syndrome coronavirus 2 (SARS-CoV-2). Elevated IL-6 secretion has been consistently reported in several studies of COVID-19 and has been linked with disease severity. For example, a recent study shows that IL-6 is an effective marker in predicting respiratory failure (Herold T et al. (2020) BMJ [Preprint, 10 Apr. 2020]).

In one aspect, the present invention provides a method for treating or preventing COVID-19 in a subject, the method comprising administering to the subject a therapeutically effective dose of a cytokine inhibitor (e.g. IL-6 inhibitor), wherein the cytokine inhibitor (e.g. IL-6 inhibitor) is pomalidomide, iberdomide, lenalidomide, avadomide or Compound 1, wherein the therapeutically effective dose is a dose sufficient to reduce or prevent the development of COVID-19 in the subject.

### T Cell Engagers

In some embodiments, the cytokine-related adverse event or disease (e.g. CRS) is caused by a T cell engaging therapy.

As used herein, the term “T cell engager” or “T-cell engaging therapy” refers to any binding molecule or polypeptide which is capable of redirecting one or more T cells so as to trigger activation and/or proliferation of said T cells.

In preferred embodiments of any aspect of the invention, the T cell engager specifically binds to an antigen that promotes activation of one or more T cells (a “T cell antigen”). In some embodiments of any aspect of the invention, the T cell engager is an antibody which specifically binds to a T cell antigen (a “T cell engaging antibody”), preferably wherein the T cell engaging antibody is multispecific, preferably bispecific. In alternative embodiments, the T cell engager comprises a chimeric antigen receptor (“CAR”) which specifically binds to a T cell antigen, preferably wherein the CAR is expressed on a T cell (“CAR T cell”).

Thus, in some embodiments, T cell engager is a multispecific antibody that specifically binds to a target antigen (e.g. cancer antigen) and to an antigen that promotes activation of one or more T cells (e.g. CD3), a chimeric antigen receptor (CAR) directed to a target antigen (e.g. cancer antigen), or a T cell expressing at least one CAR directed to a target antigen (e.g. cancer antigen).

In preferred embodiments of any aspect of the invention, the T cell engager directs one or more T cells to cancer cells. Without being bound by theory, recruitment of one or more T cells to the cancer cells may result in activation and/or proliferation of the T cells at the site of the cancer, which may result in a large and/or rapid secretion of cytokines (e.g. proinflammatory cytokines) from the T cells, other immune cells and/or bystander cells e.g. myeloid cells.

The cancer cells may be cells of a haematological cancer, a solid tumor, a metastatic cancer, soft tissue tumor, metastatic lesion, or a combination thereof. In preferred embodiments, the cancer cells are malignant B cells or plasma cells of a haematological cancer selected from multiple myeloma, acute lymphoblastic leukemia (ALL), chronic lymphocytic leukemia, a non-Hodgkins lymphoma (e.g., Burkitt’s lymphoma, chronic lymphocytic leukemia/small lymphocytic lymphoma (CLL/SLL), diffuse large B cell lymphoma (DLBCL), follicular lymphoma, immunoblastic large cell lymphoma, precursor B-lymphoblastic lymphoma, or mantle cell lymphoma), marginal zone lymphoma or plasma cell leukemia. In preferred embodiments, the cancer cells are malignant B cells or plasma cells of multiple myeloma, diffuse large B cell lymphoma (DLBCL) or plasma cell leukemia.

The cancer cells may express one or more of the following antigens on their surface: CD138, BCMA, CD81, CD19, CD45, CD56, CD319, CD137, FcRH5, CD117 and/or GPCR5d.

In preferred embodiments of any aspect of the invention, the T cell engager directs one or more T cells to cancer antigen-expressing cells.

In particularly preferred embodiments of any aspect of the invention, the T cell engager specifically binds to a cancer antigen. In some embodiments, the T cell engager is a multispecific (e.g. bispecific) antibody which specifically binds to a cancer antigen (e.g. BCMA) and to an antigen that promotes activation of one or more T cells. In alternative embodiments, the T cell engager is a chimeric antigen receptor (CAR) directed to a cancer antigen (e.g. BCMA), or a T cell expressing at least one CAR directed to the cancer antigen (e.g. BCMA).

The term “cancer antigen” as used herein, refers to a molecule (typically a protein, carbohydrate or lipid) that is expressed on the surface of a cancer cell, either entirely or as a fragment (e.g. MHC/peptide). In preferred embodiments, the cancer antigen is a human cancer antigen that is expressed on the surface of a human cancer cell, preferably a malignant B cell or plasma cell.

In some embodiments, the cancer antigen is CD19, CD20, GPCR5d, FcRH5, ROR1 or BCMA. In particularly preferred embodiments, the cancer antigen is BCMA. Alternatively, the T cell engager may specifically bind to a member of the BCMA axis, such as BAFF or APRIL.

### T-Cell Engaging Antibodies

As used herein, a “T-cell engaging antibody” is an antibody which specifically binds to an antigen that promotes activation of one or more T cells (“T cell antigen”). The T cell antigen may be selected from the group consisting of CD3, TCRα, TCRβ, TCRy, TCRζ, ICOS, CD28, CD27, HVEM, LIGHT, CD40, 4-1BB, OX40, DR3, GITR, CD30, TIM1, SLAM, CD2, or CD226. In preferred embodiments, the antigen that promotes activation of one or more T cells is CD3. Accordingly, in preferred embodiments, the multispecific antibodies of the invention bind to CD3.

In preferred embodiments, the T-cell engaging antibody comprises an antibody specifically binding to CD3, or an antigen-binding fragment thereof.

The term “CD3” refers to the human CD3 protein multi-subunit complex. The CD3 protein multi-subunit complex is composed to 6 distinctive polypeptide chains. Thus the term includes a CD3γ chain (SwissProt P09693), a CD3δ chain (SwissProt P04234), two CD3_{ε} chains (SwissProt P07766), and one CD3ζ chain homodimer (SwissProt 20963), and which is associated with the T cell receptor α and β chain. The term encompasses “full-length,” unprocessed CD3, as well as any CD3 variant, isoform and species homolog which is naturally expressed by cells (including T cells) or can be expressed on cells transfected with genes or cDNA encoding those polypeptides.

The term “specifically binding to CD3” refers to an antibody that is capable of binding to the defined target with sufficient affinity such that the antibody is useful as a therapeutic agent in targeting CD3. The multispecific antibodies of the invention can be analysed by SPR, e.g. Biacore®, for binding to CD3. In some embodiments, the antibody specifically binding to CD3 does not bind to other antigens, or does not bind to other antigens with sufficient affinity to produce a physiological effect.

In some embodiments, the antibody specifically binding to CD3 binds to human CD3 with a dissociation constant (K_{D}) of about 10⁻⁷ M or less, a K_{D} of about 10⁻⁸ M or less, a K_{D} of about 10⁻⁹ M or less, a K_{D} of about 10⁻¹⁰ M or less, a K_{D} of about 10⁻¹¹ M or less, or a K_{D} of about 10⁻¹² M or less, as determined by a surface plasmon resonance assay, preferably measured using Biacore 8 K at 25° C. In preferred embodiments, the antibody binds to human CD3 with a dissociation constant (K_{D}) of about 10⁻⁸ M or less.

Examples of anti-CD3 antibodies include OKT3, TR66, APA 1/1, SP34, CH2527, WT31, 7D6, UCHT-1, Leu-4, BC-3, H2C, HuM291 (visilizumab), Hu291 (PDL), ChAglyCD3 (Otelixizumab), hOKT3γl(Ala-Ala) (Teplizumab) and NI-0401 (Foralumab).

The first anti-CD3 antibody generated was OKT3 (muromonab-CD3), a murine antibody binding to the CD3ε domain. Subsequent anti-CD3 antibodies include humanized or human antibodies, and engineered antibodies, for example antibodies comprising modified Fc regions.

Anti-CD3 antibodies may recognise an epitope on a single polypeptide chain, for example APA 1/1 or SP34 (Yang SJ, The Journal of Immunology (1986) 137; 1097-1100), or a conformational epitope located on two or more subunits of CD3, for example WT31, 7D6, UCHT-1 (see WO2000041474) and Leu-4. Clinical trials have been carried out using several anti-CD3 antibodies, including BC-3 (Anasetti C et al. (1992) Transplantation. 54(5): 844-851) and H2C (WO2008119567A2). Anti-CD3 antibodies in clinical development include HuM291 (visilizumab) (Norman DJ et al. (2000) Transplantation. 70(12): 1707-1712) Hu291 (PDL), ChAglyCD3 (Otelixizumab) (H Waldmann), hOKT3γl(Ala-Ala) (Teplizumab) (J Bluestone and Johnson and Johnson) and (NI-0401) Foralumab.

Any anti-CD3 antibody or antigen-binding fragment thereof may be suitable for use in the T-cell engaging antibodies of the present invention. For example, the T-cell engaging antibodies may comprise an anti-CD3 antibody selected from OKT3, TR66, APA 1/1, SP34, CH2527, WT31, 7D6, UCHT-1, Leu-4, BC-3, H2C, HuM291 (visilizumab), Hu291 (PDL), ChAglyCD3 (Otelixizumab), hOKT3γl(Ala-Ala) (Teplizumab) and NI-0401 (Foralumab). In some embodiments, the T-cell engaging antibody of the invention comprises a humanized SP34 antibody or antigen-binding fragment thereof.

In some preferred embodiments, the anti-CD3 antibody, or antigen binding fragment thereof, may be derived from SP34 and may have similar sequences and the same properties with regard to epitope binding as antibody SP34.

In some embodiments, the T-cell engaging antibody comprises an anti-CD3 antibody, or antigen binding fragment thereof, comprising a variable domain VH comprising the heavy chain CDRs of SEQ ID NOs: 1, 2 and 3 as respectively heavy chain CDR1H, CDR2H and CDR3H and a variable domain VL comprising the light chain CDRs of SEQ ID NOs: 4, 5 and 6 as respectively light chain CDR1L, CDR2L and CDR3L.

In some embodiments, the T-cell engaging antibody comprises an anti-CD3 antibody, or antigen binding fragment thereof, comprising the variable domains of SEQ ID NO:7 (VH) and SEQ ID NO: 8 (VL).

n some embodiments, the T-cell engaging antibody comprises an anti-CD3 antibody, or antigen binding fragment thereof, comprising a variable region VH comprising an amino acid sequence that is at least 75% identical, at least 90% identical, at least 95% identical or identical to the amino acid sequence of SEQ ID NO: 7 and a variable region VL comprising an amino acid sequence that is at least 75% identical, at least 90% identical, at least 95% identical, or identical to the amino acid sequence of SEQ ID NO: 8.

In some embodiments, the T-cell engaging antibody is a multispecific antibody. In preferred embodiments, the multispecific antibody specifically binds to one or more cancer antigen (e.g. BCMA) and to an antigen that promotes activation of one or more T cells (e.g. CD3).

In preferred embodiments, the T-cell engaging antibody is a bispecific antibody. In particularly preferred embodiments, the bispecific antibody specifically binds to a cancer antigen (e.g. BCMA) and to an antigen that promotes activation of one or more T cells (e.g. CD3).

### Antibody Definitions

The term “antibody” herein encompasses various antibody structures, including but not limited to monoclonal antibodies, polyclonal antibodies, multispecific antibodies (e.g., bispecific antibodies), and antibody fragments so long as they exhibit the desired antigen-binding activity.

A “heavy chain” comprises a heavy chain variable region (abbreviated herein as “VH”) and a heavy chain constant region (abbreviated herein as “CH”). The heavy chain constant region comprises the heavy chain constant domains CH1, CH2 and CH3 (antibody classes IgA, IgD, and IgG) and optionally the heavy chain constant domain CH4 (antibody classes IgE and IgM).

A “light chain” comprises a light chain variable domain (abbreviated herein as “VL”) and a light chain constant domain (abbreviated herein as “CL”). The variable regions VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The “constant domains” of the heavy chain and of the light chain are not involved directly in binding of an antibody to a target but exhibit various effector functions.

Binding between an antibody and its target antigen or epitope is mediated by the Complementarity Determining Regions (CDRs). The CDRs are regions of high sequence variability, located within the variable region of the antibody heavy chain and light chain, where they form the antigen-binding site. The CDRs are the main determinants of antigen specificity. Typically, the antibody heavy chain and light chain each comprise three CDRs which are arranged non-consecutively. The antibody heavy and light chain CDR3 regions play a particularly important role in the binding specificity/affinity of the antibodies according to the invention and therefore provide a further aspect of the invention.

The term “antigen binding fragment” as used herein incudes any naturally occurring or artificially-constructed configuration of an antigen-binding polypeptide comprising one, two or three light chain CDRs, and/or one, two or three heavy chain CDRs, wherein the polypeptide is capable of binding to the antigen. Thus, the term refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The sequence of a CDR may be identified by reference to any number system known in the art, for example, the Kabat system (Kabat EA et al. (1991) Sequences of Proteins of Immunological Interest, 5th ed. Public Health Service. National Institutes of Health. Bethesda, MD.); the Chothia system (Chothia C and Lesk AM (1987) J. Mol. Biol. 196(4): 901-917); or the IMGT system (Lefranc MP et al. (2003) Dev. Comp. Immunol. 27(1): 55-77).

TABLE 1

CDR definitions

| | Kabat | Chothia | IMGT |
|---|---|---|---|
| VH CDR1 | 31-35 | 26-32 | 27-38 |
| VH CDR2 | 50-65 | 52-56 | 56-65 |
| VH CDR3 | 95-102 | 95-102 | 105-117 |
| VL CDR1 | 24-34 | 24-34 | 27-38 |
| VL CDR2 | 50-56 | 50-56 | 56-65 |
| VL CDR3 | 89-97 | 89-97 | 105-117 |

For heavy chain constant region amino acid positions discussed in the invention, numbering is according to the EU index first described in Edelman GM et al. (1969) Proc. Natl. Acad. Sci. USA. 63(1): 78-85. The EU numbering of Edelman is also set forth in Kabat et al. (1991) (supra). Thus, the terms “EU index as set forth in Kabat”, “EU Index”. “EU index of Kabat” or “EU numbering” in the context of the heavy chain refers to the residue numbering system based on the human lgG1 EU antibody of Edelman et al. as set forth in Kabat et al. (1991). The numbering system used for the light chain constant region amino acid sequence is similarly set forth in Kabat et al. (supra). Thus, as used herein, “numbered according to Kabat” refers to the Kabat set forth in Kabat et al. (supra).

The antibodies of the invention and antigen-binding fragments thereof may be derived from any species by recombinant means. For example, the antibodies or antigen-binding fragments may be mouse, rat, goat, horse, swine, bovine, chicken, rabbit, camelid, donkey, human, or chimeric versions thereof. For use in administration to humans, non-human derived antibodies or antigen-binding fragments may be genetically or structurally altered to be less antigenic upon administration to the human patient.

Especially preferred are human or humanized antibodies, especially as recombinant human or humanized antibodies.

The term “humanized antibody” refers to antibodies in which the framework or “complementarity determining regions” (CDRs) have been modified to comprise the CDR of an immunoglobulin of different specificity as compared to that of the parent immunoglobulin. For example, a murine CDR may be grafted into the framework region of a human antibody to prepare the “humanized antibody.” See, e.g.., Riechmann L et al. (1988) Nature. 332: 323-327; and Neuberger MS et al. (1985) Nature. 314: 268-270. In some embodiments, “humanized antibodies” are those in which the constant region has been additionally modified or changed from that of the original antibody to generate the properties of the antibodies according to the invention, especially in regard to Clq binding and/or Fc receptor (FcR) binding.

The term “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties of the antibodies according to the invention, especially in regard to Clq binding and/or Fc receptor (FcR) binding. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involving conventional recombinant DNA and gene transfection techniques are well known in the art. See, e.g., Morrison SL et al. (1984) Proc. Natl. Acad. Sci. USA. 81(21): 6851-6855; U.S. Pat. Nos. 5,202,238 and 5,204,244.

The terms “Fc region” and “Fc” are used interchangeably herein and refer to the portion of a native immunoglobulin that is formed by two Fc chains. Each “Fc chain” comprises a constant domain CH2 and a constant domain CH3. Each Fc chain may also comprise a hinge region. A native Fc region is homodimeric. In some embodiments, the Fc region may contain modifications to enforce Fc heterodimerization.

The term “Fc part” refers to the portion of an antibody of the invention, or antigen binding fragment thereof, which corresponds to the Fc region.

There are five major classes of heavy chain constant region, classified as IgA, IgG, IgD, IgE and IgM, each with characteristic effector functions designated by isotype. For example, IgG is separated into four subclasses known as IgGl, IgG2, IgG3, and IgG4. Ig molecules interact with multiple classes of cellular receptors. For example, IgG molecules interact with three classes of Fcy receptors (FcyR) specific for the IgG class of antibody, namely FcyRI, FcyRII, and FcyRIII. The important sequences for the binding of IgG to the FcyR receptors have been reported to be located in the CH2 and CH3 domains.

The antibodies of the invention or antigen-binding fragments thereof may be any isotype, i.e. IgA, IgD, IgE, IgG and IgM, and synthetic multimers of the four-chain immunoglobulin (Ig) structure. In preferred embodiments, the antibodies or antigen-binding fragments thereof are IgG isotype. The antibodies or antigen-binding fragments can be any IgG subclass, for example IgG1, IgG2, IgG3, or IgG4 isotype. In preferred embodiments, the antibodies or antigen-binding fragments thereof are of an IgGl isotype.

In some embodiments, the antibodies comprise a heavy chain constant region that is of IgG isotype. In some embodiments, the antibodies comprise a portion of a heavy chain constant region that is of IgG isotype. In some embodiments, the IgG constant region or portion thereof is an IgG1, IgG2, IgG3, or IgG4 constant region. Preferably, the IgG constant region or portion thereof is an IgG1 constant region.

The antibodies of the invention or antigen-binding fragments thereof may comprise a lambda light chain or a kappa light chain.

In preferred embodiments, the antibodies or antigen-binding fragments thereof comprise a light chain that is a kappa light chain. In some embodiments, the antibody or antigen-binding fragment comprises a light chain comprising a light chain constant region (CL) that is a kappa constant region.

In some embodiments, the antibody comprises a light chain comprising a light chain variable region (VL) that is a kappa variable region. Preferably, the kappa light chain comprises a VL that is a kappa VL and a CL that is a kappa CL.

Alternatively, the antibodies or antigen-binding fragments thereof may comprise a light chain that is a lambda light chain. In some embodiments, the antibody or antigen-binding fragment comprises a light chain comprising a light chain constant region (CL) that is a lambda constant region. In some embodiments, the antibody comprises a light chain comprising a light chain variable region (VL) that is a lambda variable region.

Engineered antibodies and antigen-binding fragments thereof include those in which modifications have been made to framework residues within the VH and/or VL. Such modifications may improve the properties of the antibody, for example to decrease the immunogenicity of the antibody and/or improve antibody production and purification.

Antibodies and antigen-binding fragments thereof disclosed herein can be further modified using conventional techniques known in the art, for example, by using amino acid deletion(s), insertion(s), substitution(s), addition(s), and/or recombination(s) and/or any other modification(s) known in the art, either alone or in combination. Methods for introducing such modifications in the DNA sequence underlying the amino acid sequence of an immunoglobulin chain arc well known to the person skilled in the art.

The antibodies of the invention and antigen-binding fragments thereof also include derivatives that are modified (e.g., by the covalent attachment of any type of molecule to the antibody) such that covalent attachment does not prevent the antibody from binding to its epitope, or otherwise impair the biological activity of the antibody. Examples of suitable derivatives include, but are not limited to fucosylated antibodies, glycosylated antibodies, acetylated antibodies, PEGylated antibodies, phosphorylated antibodies, and amidated antibodies.

Minor variations in the amino acid sequences of antibodies of the invention are contemplated as being encompassed by the present invention, providing that the variations in the amino acid sequence(s) maintain at least 75%, more preferably at least 80%, at least 90%, at least 95%, and most preferably at least 99% sequence identity to the antibody of the invention or antigen-binding fragment thereof as defined anywhere herein.

Antibodies of the invention may include variants in which amino acid residues from one species are substituted for the corresponding residue in another species, either at the conserved or non-conserved positions. In one embodiment, amino acid residues at non-conserved positions are substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in an antibody of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

### Chimeric Antigen Receptors

Chimeric antigen receptors (CARs) are artificial receptors that are expressed on the surface of modified immune cells, for example, T cells, in order to direct the immune cell to a target cell. In preferred embodiments, the CAR directs the immune cell (e.g. T cell) to a cancer cell (e.g. malignant B cell or plasma cell of a haematological cancer). In particularly preferred embodiments, the CAR specifically binds to one or more cancer antigens (e.g. BCMA).

Generally, CARs comprise an extracellular antigen binding domain, a transmembrane domain; and an intracellular signalling domain. In certain embodiments, once the extracellular domain binds to a target antigen, such as a cancer antigen (e.g. BCMA), a signal is generated via the intracellular signalling domain that activates the immune cell, e.g. to target and kill a cell expressing the target antigen.

In certain embodiments, the extracellular domain comprises a receptor, or a portion of a receptor, that binds to the target antigen (e.g. BCMA). In certain embodiments, the extracellular domain comprises, or is, an antibody or an antigen-binding portion thereof that binds to the target antigen (e.g. an anti-BCMA antibody described herein). In specific embodiments, the extracellular domain comprises, or is, a single chain Fv (scFv) domain. The single-chain Fv domain can comprise, for example, a VL linked to VH by a flexible linker, wherein said VL and VH are from an antibody that binds the target antigen.

The transmembrane domain can be any transmembrane domain derived or obtained from any molecule known in the art. In specific embodiments, the transmembrane domain can be obtained or derived from CD8, CD28, a cytokine receptor, an interleukin receptor, a growth factor receptor, or the like.

In a preferred embodiment, the transmembrane domain is obtained or derived from a human CD8α molecule or CD28 molecule. CD8 is a transmembrane glycoprotein that serves as a co-receptor for the T-cell receptor (TCR) and is expressed primarily on the surface of cytotoxic T-cells. The most common form of CD8 exists as a dimer composed of a CD8 alpha and CD8 beta chain. CD28 is expressed on T-cells and provides co-stimulatory signals required for T-cell activation. CD28 is the receptor for CD80 (B7.1) and CD86 (B7.2).

In certain embodiments, the extracellular domain of the CAR is joined to the transmembrane domain of the polypeptide by a linker, spacer domain or hinge polypeptide e.g., a sequence from CD28 or a sequence from CTLA4. The spacer domain may be derived from any natural, synthetic, semi-synthetic or recombinant source. For example, the spacer domain can be derived or obtained from a portion of an immunoglobulin (such as IgGl or IgG4), including, but not limited to, one or more constant regions (e.g. CH2 and CH3) or a hinge region, or modified versions thereof.

The intercellular signalling domain can be obtained or derived from any intracellular signalling molecule known in the art. In certain embodiments, the intracellular domain is obtained or derived from a protein that is expressed on the surface of T cells and triggers activation and/or proliferation of said T cells. In specific embodiments, the intracellular signalling domain is obtained or derived from CD3 zeta (CD3ζ or modified versions thereof. In other embodiments, the intracellular domain is obtained or derived from a lymphocyte receptor chain, a TCR/CD3 complex protein, an Fc receptor subunit or an IL-2 receptor subunit. In some embodiments, the intracellular domain additionally comprises one or more co-stimulatory domains or motifs. The one or more co-stimulatory domains or motifs can be obtained or derived from, CD28, OX40 (CD134), 4-1BB (CD137), CD27, or a co-stimulatory inducible T-cell costimulatory (ICOS) polypeptide, or other costimulatory domain or motif, or any combination thereof. In some embodiments, the CD3 zeta, CD28, 4-1BB, OX40, and/or CD27 are human.

Such a CAR is usually transferred by using a vector, preferably a retroviral vector, comprising the sequence encoding said CAR, into an immune effector cell. The modified immune cells expressing the CAR can be T cells (e.g., CD4+ T cells, CD8+ T cells or cytotoxic T lymphocytes) for which herein the term “a CAR T-cell” is used. Such CAR T cells are also provided by the present invention.

T cells used in the compositions and methods provided herein may be naive T lymphocytes or MHC-restricted T lymphocytes. In certain embodiments, the T cells are tumor infiltrating lymphocytes (TILs). In certain embodiments, the T cells have been isolated from a tumor biopsy, or are or have been expanded from T cells isolated from a tumor biopsy. In certain other embodiments, the T cells have been isolated from, or are expanded from T cells isolated from, peripheral blood, cord blood, or lymph. T cells to be used to generate modified T cells expressing a CAR can be isolated using art-accepted, routine methods, e.g., blood collection followed by apheresis and optionally antibody-mediated cell isolation or sorting.

The modified T cells are preferably autologous to an individual to whom the modified T cells are to be administered. In certain other embodiments, the modified T cells are allogeneic to an individual to whom the modified T cells are to be administered. Where allogeneic T cells are used to prepare modified T cells, it is preferable to select T cells that will reduce the possibility of graft-versus-host disease (GVHD) in the individual. For example, in certain embodiments, virus-specific T cells are selected for preparation of modified T cells and are expected to have a greatly reduced native capacity to bind to, and thus become activated by, any recipient antigens. In certain embodiments, recipient-mediated rejection of allogeneic T cells can be reduced by coadministration to the host of one or more immunosuppressive agents, e.g., cyclosporine, tacrolimus, sirolimus, cyclophosphamide, or the like.

T cells, e.g., unmodified T lymphocytes, or T cells expressing CD3 and CD28, or comprising a polypeptide comprising a CD3ζ, signalling domain and a CD28 co-stimulatory domain, can be expanded using antibodies to CD3 and CD28, e.g., antibodies attached to beads; see, e.g., U.S. Pat. Nos. 5,948,893; 6,534,055; 6,352,694; 6,692,964; 6,887,466; and 6,905,681.

The modified T cells can optionally comprise a “suicide gene” or “safety switch” that enables killing of substantially all of the modified T cells when desired. For example, the modified T cells, in certain embodiments, can comprise an HSV thymidine kinase gene (HSV-TK), which causes death of the modified T cells upon contact with gancyclovir. In another embodiment, the modified T cells comprise an inducible caspase, e.g., an inducible caspase 9 (icaspase9), e.g., a fusion protein between caspase 9 and human FK506 binding protein allowing for dimerization using a specific small molecule pharmaceutical. See Straathof KC et al. (2005) Blood 105(11): 4247-4254.

### BCMA Therapeutic Agent

As used herein, the term “BCMA therapeutic agent” refers to a binding molecule or polypeptide which specifically binds to BCMA with sufficient affinity such that the binding molecule or polypeptide is useful as a therapeutic agent in targeting BCMA. In preferred embodiments, the BCMA therapeutic agent is a T cell engager. Such T cell engagers are capable of redirecting one or more T cells to BCMA-expressing target cells.

The term “BCMA” as used herein relates to human B cell maturation antigen, also known as BCMA; TR17_HUMAN, TNFRSF17 (UniProt Q02223), which is a member of the tumor necrosis factor (TNF) receptor superfamily that is preferentially expressed in differentiated plasma cells. The extracellular domain of BCMA consists according to UniProt of amino acids 1-54 (or 5-51). BCMA is a transmembrane glycoprotein essential for the maturation and survival of multiple myeloma cells.

As used herein, a “disorder associated with BCMA expression” is a disorder in which patients have aberrant or enhanced BCMA expression. Disorders associated with BCMA expression include plasma cell disorders or B cell disorders such as multiple myeloma, chronic lymphocytic leukemia, or a non-Hodgkins lymphoma (e.g., Burkitt’s lymphoma, chronic lymphocytic leukemia/small lymphocytic lymphoma (CLL/SLL), diffuse large B cell lymphoma (DLBCL), follicular lymphoma, immunoblastic large cell lymphoma, precursor B-lymphoblastic lymphoma, or mantle cell lymphoma), marginal zone lymphoma, plasma cell leukemia, or IgG4-related disease. In preferred embodiments, the disorder associated with BCMA expression is multiple myeloma, diffuse large B cell lymphoma (DLBCL) or plasma cell leukemia.

In some embodiments, the disorder associated with BCMA expression is relapsed or refractory, e.g. relapsed or refractory multiple myeloma, relapsed or refractory diffuse large B cell lymphoma (DLBCL) or relapsed or refractory plasma cell leukemia. In alternative embodiments, the disorder associated with BCMA expression is newly diagnosed (i.e. has not yet undergone treatment), e.g. newly diagnosed multiple myeloma, newly diagnosed diffuse large B cell lymphoma (DLBCL) or newly diagnosed plasma cell leukemia.

In particularly preferred embodiments, the disorder associated with BCMA expression is multiple myeloma, e.g., high-risk multiple myeloma or relapsed and refractory multiple myeloma. In some embodiments, the high risk multiple myeloma is R-ISS stage III disease and/or a disease characterized by early relapse (e.g., progressive disease within 12 months since the date of last treatment regimen, such as last treatment regimen with a proteasome inhibitor, an immunomodulatory agent and/or dexamethasone).

In preferred embodiments of any aspect of the invention, the BCMA therapeutic agent comprises an anti-BCMA antibody, or antigen binding fragment thereof.

The terms “antibody against BCMA”, “anti BCMA antibody” or “an antibody that binds to BCMA” as used herein relate to an antibody specifically binding to the extracellular domain of BCMA. In some embodiments, an antibody specifically binding to BCMA does not bind to other antigens or does not bind to other antigens with sufficient affinity to produce a physiological effect.

In some embodiments, the extent of binding of an anti-BCMA antibody to an unrelated, non-BCMA protein is about 10-fold preferably >100-fold less than the binding of the antibody to BCMA as measured, e.g., by surface plasmon resonance (SPR) e.g. Biacore®, enzyme-linked immunosorbent (ELISA) or flow cytometry (FACS). In one embodiment the antibody that binds to BCMA has a dissociation constant (Kd) of 10⁻⁸ M or less, preferably from 10⁻⁸ M to 10⁻¹³ M, preferably from 10⁻⁹ M to 10⁻¹³ M.

In one embodiment, the anti-BCMA antibody binds to an epitope of BCMA that is conserved among BCMA from different species, preferably among human and cynomolgus, and in addition preferably also to mouse and rat BCMA.

Preferably the anti-BCMA antibody specifically binds to a group of BCMA, consisting of human BCMA and BCMA of non-human mammalian origin, preferably BCMA from cynomolgus, mouse and/or rat. Anti-BCMA antibodies are analyzed by ELISA for binding to human BCMA using plate-bound BCMA. For this assay, an amount of plate-bound BCMA preferably 1.5 µg/mL and concentration(s) ranging from 0.1 pM to 200 nM of anti-BCMA antibody are used.

In preferred embodiments, the BCMA therapeutic agent is a T cell engager as described herein.

Thus, in some embodiments, the BCMA therapeutic agent is a multispecific (e.g. bispecific) antibody that specifically binds to BCMA and to an antigen that promotes activation of one or more T cells (e.g. CD3), a chimeric antigen receptor (CAR) directed to BCMA, or a T cell expressing at least one CAR directed to BCMA.

In some preferred embodiments the BCMA therapeutic agent is a multispecific (e.g. bispecific) antibody which specifically binds to BCMA and to an antigen that promotes activation of one or more T cells (e.g. CD3). In particularly preferred embodiments, the multispecific (e.g. bispecific) antibody comprises an anti-BCMA antibody described herein, or antigen-binding fragment thereof.

In alternative preferred embodiments, the BCMA therapeutic agent comprises a chimeric antigen receptor (CAR) directed to BCMA, or a T cell expressing at least one CAR (“CAR T cell”) directed to BCMA. In some embodiments, the extracellular domain of the CAR directed to BCMA comprises a receptor, or a portion of a receptor, that binds to BCMA. In some embodiments, the extracellular domain of the CAR directed to BCMA comprises an anti-BCMA antibody described herein, e.g., a single chain Fv (scFv), or antigen binding fragment thereof.

In alternative embodiments, the BCMA therapeutic agent is an antibody-drug conjugate (ADC). The term “antibody drug conjugate” or “conjugated antibody” as used herein refers to an antibody which specifically binds to an antigen (e.g. BCMA), and is conjugated with a therapeutic agent, e.g. with a cytotoxic agent or radiolabel.

In some embodiments, the ADC comprises an anti-BCMA antibody, or antigen-binding fragment thereof, described herein. In some embodiments the antibody-drug conjugate comprises a maytansinoid, preferably wherein said maytansinoid is a noncleavable DM1-like maytansinoid. In some embodiments, the antibody-drug conjugate is GSK2857916, AMG224 or CC99712.

### Dosage Regimens

In some embodiments, the cytokine inhibitor (e.g. IL-6 inhibitor) is administered at a dose sufficient to prevent or reduce the development of CRS in the subject. The present inventors have recognized that administration of the cytokine inhibitors (e.g. IL-6 inhibitors) described herein to a subject may increase the safety of a therapeutic agent that has caused or is likely to cause CRS by attenuating cytokine release (e.g. pro-inflammatory cytokine release). Without being bound by theory, it is thought that the cytokine inhibitor suppresses pro-inflammatory cytokines from myeloid cells (e.g. macrophages and/or monocytes), and thus may prevent or reduce the development of CRS or treat CRS in the subject. Alternatively or in addition, administration of the cytokine inhibitors (e.g. IL-6 inhibitors) described herein may allow the therapeutic agent that has caused or is likely to cause CRS to be administered at an increased dose due to reduced toxicity as compared to administration without the cytokine inhibitor, and thereby may increase the therapeutic index of the therapeutic agent.

The cytokine inhibitor (e.g. IL-6 inhibitor) and the therapeutic agent (e.g. BCMA therapeutic agent) may be administered concurrently, at overlapping timepoints or at different timepoints. In some embodiments, the cytokine inhibitor (e.g. IL-6 inhibitor) is administered prior to the therapeutic agent (e.g. prior to the first dose of the therapeutic agent). In alternative embodiments, the cytokine inhibitor (e.g. IL-6 inhibitor) is administered after the therapeutic agent (e.g. after the first dose of the therapeutic agent).

In some embodiments of any aspect of the invention, the cytokine inhibitor (e.g. IL-6 inhibitor) is administered before (e.g. within 12 or 24 hours before) administration of the BCMA therapeutic agent, on the same day as administration of the BCMA therapeutic agent or after (e.g. within 12 or 24 hours after) administration of the BCMA therapeutic agent. In some embodiments of any aspect of the invention, the cytokine inhibitor (e.g. IL-6 inhibitor) is administered within 12 hours after diagnosis of CRS.

### A) Lead With Cytokine Inhibitor

In some embodiments, the cytokine inhibitor (e.g. IL-6 inhibitor) is administered to the subject as one or more doses, wherein at least one dose of the cytokine inhibitor (e.g. IL-6 inhibitor) is administered before administration of a first dose of the therapeutic agent (e.g. T cell engager). Without being bound by theory, pre-treatment with the cytokine inhibitor (e.g. IL-6 inhibitor) may prevent or reduce the development of CRS associated with administration of the therapeutic agent (e.g. T cell engager). In addition, in embodiments in which the therapeutic agent is a T cell engager (e.g. multispecific T-cell engaging antibody, CAR or CAR T cell), administration of the cytokine inhibitor (e.g. IL-6 inhibitor) described herein may increase efficacy of the therapeutic agent e.g. by potentiating T cell activation.

In some embodiments, one or more doses of the cytokine inhibitor (e.g. IL-6 inhibitor) is administered to the subject 1-28 days, e.g. 1, 2, 3, 4, 5, 6, 7, 8, 9, 10, 11, 12, 13,14, 15, 16, 17, 18, 19, 20, 21, 22, 23, 24, 25, 26, 27 or 28 days, before the first dose of the therapeutic agent (e.g. T cell engager). In preferred embodiments, one or more doses of the cytokine inhibitor (e.g. IL-6 inhibitor) is administered to the subject 7 days before the first dose of the therapeutic agent (e.g. T cell engager).

In some embodiments, two or more doses (e.g. two, three, four, five, six or seven doses) of the cytokine inhibitor (e.g. IL-6 inhibitor) are administered to the subject 1-28 days, e.g. 1, 2, 3, 4, 5, 6, 7, 8, 9, 10, 11, 12, 13, 14, 15, 16, 17, 18, 19, 20, 21, 22, 23, 24, 25, 26, 27 or 28 days, before the first dose of the therapeutic agent (e.g. T cell engager). In some embodiments, a first dose of the cytokine inhibitor (e.g. IL-6 inhibitor) is administered to the subject 7 days before the first dose of the therapeutic agent (e.g. T cell engager), and one or more further doses of the cytokine inhibitor (e.g. IL-6 inhibitor) is administered to the subject before (e.g. 1, 2, 3, 4, 5 or 6 days before) administration of the first dose of the therapeutic agent (e.g. T cell engager). For example, six further doses of the cytokine inhibitor (e.g. IL-6 inhibitor) may be administered to the subject 6, 5, 4, 3, 2 and 1 days before administration of the first dose of the therapeutic agent (e.g. T cell engager).

In some embodiments of any aspect of the invention, the cytokine inhibitor (e.g. IL-6 inhibitor) is administered to the subject as one or more doses, wherein at least one dose of the cytokine inhibitor (e.g. IL-6 inhibitor) is administered on the same day as administration of a first dose of the therapeutic agent (e.g. T cell engager). Accordingly, in some embodiments, the cytokine inhibitor (e.g. IL-6 inhibitor) is administered to the subject as two or more doses, wherein the two or more doses comprises:
(i) one or more doses 1-28 days, e.g. 1, 2, 3, 4, 5, 6, 7, 8, 9, 10, 11, 12, 13, 14, 15, 16, 17, 18, 19, 20, 21, 22, 23, 24, 25, 26, 27 or 28 days, preferably 7 days, before the first dose of the therapeutic agent (e.g. T cell engager); and
(ii) at least one dose on the same day as administration of the first dose of the therapeutic agent (e.g. T cell engager).

For example, the cytokine inhibitor (e.g. IL-6 inhibitor) may be administered to the subject as seven doses 7, 6, 5, 4, 3, 2 and 1 days before administration of the first dose of the therapeutic agent (e.g. T cell engager), and at least one dose on the same day as administration of the first dose of the therapeutic agent (e.g. T cell engager).

In alternative embodiments of any aspect of the invention, the cytokine inhibitor (e.g. IL-6 inhibitor) is not administered to the subject on the same day as administration of a first dose of the therapeutic agent (e.g. T cell engager). Without being bound by theory, such embodiments may reduce the risk of adverse events such as neutropenia and/or infection.

In some embodiments of any aspect of the invention, one or more doses of the cytokine inhibitor (e.g. IL-6 inhibitor) is administered to the subject after administration of a first dose of the therapeutic agent (e.g. T cell engager). Without being bound by theory, post-treatment with the cytokine inhibitor (e.g. IL-6 inhibitor) may prevent or reduce development of CRS or treat CRS associated with administration of the therapeutic agent.

In some embodiments, following administration of the first dose of the therapeutic agent (e.g. T cell engager), the next dose of the cytokine inhibitor (e.g. IL-6 inhibitor) is administered to the subject 1-14 days, e.g. 1, 2, 3, 4, 5, 6, 7, 8, 9, 10, 11, 12, 13 or 14 days, after the first dose of the therapeutic agent. In embodiments in which the cytokine inhibitor (e.g. IL-6 inhibitor) is not administered to the subject on the same day as administration of the first dose of the therapeutic agent (e.g. T cell engager), the next dose of cytokine inhibitor may be administered to the subject 7-14 days (e.g. 7 days) after the first dose of the therapeutic agent. Without being bound by theory, the risk of adverse events such as neutropenia or infection is thought to diminish after 1 week of administration of the therapeutic agent.

In some embodiments of any aspect of the invention, the cytokine inhibitor (e.g. IL-6 inhibitor) is administered to the patient as a treatment comprising at least one treatment cycle. As used herein, a “treatment cycle” or “cycle” is 28 days. In some embodiments, the treatment comprises a first treatment cycle wherein the cytokine inhibitor (e.g. IL-6 inhibitor) is administered to the patient on days 1-21 and the therapeutic agent (e.g. T cell engager) is administered to the patient on days 8, 11, 15 and 22. In alternative embodiments, the treatment comprises a first treatment cycle wherein the cytokine inhibitor (e.g. IL-6 inhibitor) is administered to the patient on days 1-7 and days 15-21 and the therapeutic agent (e.g. T cell engager) is administered to the patient on days 8, 11, 15 and 22. In further alternative embodiments, the treatment comprises a first treatment cycle wherein the cytokine inhibitor (e.g. IL-6 inhibitor) is administered to the patient on days 1-21 and the therapeutic agent (e.g. T cell engager) is not administered to the patient in the treatment cycle. In further alternative embodiments, the treatment comprises a first treatment cycle wherein the cytokine inhibitor (e.g. IL-6 inhibitor) is administered to the patient on days 1-28 and the therapeutic agent (e.g. T cell engager) is not administered to the patient in the treatment cycle.

In some embodiments, the treatment comprises a second treatment cycle, wherein the cytokine inhibitor (e.g. IL-6 inhibitor) is administered to the patient on days 1-21 and the therapeutic agent (e.g. T cell engager) is administered to the patient on days 1, 8, 15 and 22. In some embodiments, the treatment comprises a third treatment cycle, optionally a fourth, fifth and sixth treatment cycle, wherein cytokine inhibitor (e.g. IL-6 inhibitor) is administered to the patient on days 1-21 and the therapeutic agent (e.g. T cell engager) is administered to the patient on days 1, 8, 15 and 22. In some embodiments, the patient continues to receive treatment (e.g. for the rest of their lives).

In alternative embodiments, the treatment comprises a second treatment cycle, wherein the cytokine inhibitor (e.g. IL-6 inhibitor) is administered to the patient on days 1-7 and 15-21 and the therapeutic agent (e.g. T cell engager) is administered to the patient on days 1, 8, 15 and 22. In some embodiments, the treatment comprises a third treatment cycle, optionally a fourth, fifth and sixth treatment cycle, wherein cytokine inhibitor (e.g. IL-6 inhibitor) is administered to the patient on days 1-7 and 15-21 and the therapeutic agent (e.g. T cell engager) is administered to the patient on days 1, 8, 15 and 22. In some embodiments, the patient continues to receive treatment (e.g. for the rest of their lives).

In preferred embodiments, the T cell engager is a BCMA therapeutic agent (e.g. 42-TCBcv). The BCMA therapeutic agent (e.g. 42-TCBcv) may be administered to the patient (e.g. multiple myeloma patient) in accordance with the regimen set out in Table 2 or Table 3.

TABLE 2

| | | |
|---|---|---|
| | Cycle 1 | Cycle 2+ |
| Cytokine inhibitor (e.g. Compound 1 or iberdomide) | Days 1-21 | Days 1-21 |
| BCMA therapeutic agent (e.g. 42-TCBcv) | Days 8, 11, 15, 22 | Days 1, 8, 15, 22 |

TABLE 3

| | | |
|---|---|---|
| | Cycle 1 | Cycle 2+ |
| Cytokine inhibitor (e.g. Compound 1 or iberdomide) | Days 1-7, 15-21 | Days 1-21 |
| BCMA therapeutic agent (e.g. 42-TCBcv) | Days 8, 11, 15, 22 | Days 1, 8, 15, 22 |

In some embodiments, the cytokine inhibitor (e.g. iberdomide or Compound 1) is administered orally. In some embodiments, the one or more doses of cytokine inhibitor (e.g. iberdomide or Compound 1) is administered at a fixed dose. In some embodiments, iberdomide is administered at a fixed dose of between about 0.03 mg to about 6 mg, between about 0.1 mg to about 4 mg, between about 0.3 mg to about 2 mg, or between about 1.0 mg to about 1.6 mg, e.g. about 1.0 mg, about 1.3 mg or about 1.6 mg. In some embodiments, Compound 1 is administered at a fixed dose of between about 0.05 mg to about 5 mg, between about 0.1 mg to about 2 mg, between about 0.2 mg to about 1.6 mg, or between about 0.3 mg to about 1.0 mg, e.g. about 0.3 mg, about 0.6 mg or about 1.0 mg.

In some embodiments, the BCMA therapeutic agent is administered intravenously. In preferred embodiments, the first dose of the BCMA therapeutic agent (e.g. 42-TCBcv) is administered subcutaneously. In some embodiments, the BCMA therapeutic agent (e.g. 42-TCBcv) is administered subcutaneously in the first cycle, optionally in the first and subsequent cycles. In some embodiments, the BCMA therapeutic agent (e.g. 42-TCBcv) is administered at a dose of between about 1 mg to about 100 mg, between about 1 mg to about 75 mg, between about 1 mg to about 50 mg, between about 1 mg to about 25 mg, or between about 1 mg to about 12 mg.

In some embodiments in which the cytokine inhibitor is Compound 1 or iberdomide, the BCMA therapeutic agent (e.g. 42-TCBcv) and the cytokine inhibitor may be administered to the patient (e.g. multiple myeloma patient) in accordance with the regimen set out in Table 4, 5 or 6.

TABLE 4

| | | |
|---|---|---|
| | Cycle 1 | Cycle 2+ |
| Compound 1 at a fixed dose of about 0.3 mg, about 0.6 mg or about 1.0 mg | Days 1-21 | Days 1-21 |
| BCMA therapeutic agent (e.g. 42-TCBcv at a dose of about 1.0 mg to about 100 mg) | Days 8, 11, 15, 22 | Days 1, 8, 15, 22 |

TABLE 5

| | | |
|---|---|---|
| | Cycle 1 | Cycle 2+ |
| Iberdomide at a fixed dose of about 1.0 mg, about 1.3 mg or about 1.6 mg | Days 1-21 | Days 1-21 |
| BCMA therapeutic agent (e.g. 42-TCBcv at a dose of about 1.0 mg to about 100 mg) | Days 8, 11, 15, 22 | Days 1, 8, 15, 22 |

TABLE 6

| | | |
|---|---|---|
| | Cycle 1 | Cycle 2+ |
| Compound 1 at a fixed dose of about 0.6 mg or iberdomide at a fixed dose of about 1.3 mg | Days 1-7, 15-21 | Days 1-21 |
| BCMA therapeutic agent (e.g. 42-TCBcv at a dose of about 1.0 mg to about 100 mg) | Days 8, 11, 15, 22 | Days 1, 8, 15, 22 |

In some embodiments, one or more additional therapeutic agents is administered to the patient. In preferred embodiments, a corticosteroid is administered, preferably dexamethasone. In some embodiments, the corticosteroid (e.g. dexamethasone) is administered to the patient weekly for at least the first treatment cycle, at least the first two treatment cycles, or at least the first three treatment cycles. In preferred embodiments, the corticosteroid (e.g. dexamethasone) is administered to the patient (e.g. weekly) for three treatment cycles. The dose of the corticosteroid (e.g. dexamethasone) may be about 40 mg per week (e.g. for patients up to and including 75 years old or not underweight) or about 20 mg per week (e.g. for patients 75 years and older or underweight, body mass index [BMI] <18.5). Preferably, the corticosteroid (e.g. dexamethasone) is administered orally or intravenously. In some embodiments, the corticosteroid (e.g. dexamethasone) is administered orally with the cytokine inhibitor (e.g. iberdomide or Compound 1). In some embodiments, the corticosteroid (e.g. dexamethasone) is administered intravenously on a day on which the BCMA therapeutic agent (e.g. 42-TCBcv) is administered.

In some embodiments, the BCMA therapeutic agent (e.g. 42-TCBcv), cytokine inhibitor and corticosteroid (e.g. dexamethasone) are administered to the patient (e.g. multiple myeloma patient) in accordance with the regimen set out in Table 7, 8 or 9.

TABLE 7

| | | |
|---|---|---|
| | Cycle 1 | Cycle 2+ |
| Compound 1 at a fixed dose of about 0.3 mg, about 0.6 mg or about 1.0 mg | Days 1 -2 1 | Days 1-21 |
| BCMA therapeutic agent (e.g. 42-TCBcv at a dose of about 1.0 mg to about 100 mg | Days 8, 11, 15.22 | Days 1, 8, 15, 22 |
| Corticosteroid (e.g. dexamethasone at about 40 mg / week for patients ≤75 and at about 20 mg / week for patients > 75 or underweight) | Once per week | Once per week up to the end of cycle 3 |

TABLE 8

| | | |
|---|---|---|
| | Cycle 1 | Cycle 2+ |
| Iberdomide at a fixed dose of about 1.0 mg, about 1.3 mg or about 1.6 mg | Days 1-21 | Days 1-21 |
| BCMA therapeutic agent (e.g. 42-TCBcv at a dose of about 1.0 mg to about 100 mg) | Days 8, 11, 15, 22 | Days 1, 8, 15, 22 |
| Corticosteroid (e.g. dexamethasone at about 40 mg / week for patients ≤ 75 and at about 20 mg / week for patients > 75 or underweight) | Once per week | Once per week up to the end of cycle 3 |

TABLE 9

| | | |
|---|---|---|
| | Cycle 1 | Cycle 2+ |
| Compound 1 at a fixed dose of about 0.6 mg or iberdomide at a fixed dose of about 1.3 mg | Days 1-7, 15-21 | Days 1-21 |
| BCMA therapeutic agent (e.g. 42-TCBcv at a dose of about 1.0 mg to about 100 mg) | Days 8, 11, 15, 22 | Days 1, 8, 15, 22 |
| Corticosteroid (e.g. dexamethasone at about 40 mg / week for patients ≤75 and at about 20 mg / week for patients > 75 or underweight) | Once per week | Once per week up to the end of cycle 3 |

In an aspect, there is provided a method of treating a disorder associated with BCMA expression (e.g. multiple myeloma), wherein the method comprises administering a BCMA therapeutic agent (e.g. 42-TCBcv) and a cytokine inhibitor (e.g. IL-6 inhibitor) to a subject, wherein the BCMA therapeutic agent (e.g. 42-TCBcv) and the cytokine inhibitor (e.g. IL-6 inhibitor) are administered to the subject in accordance with any one of the regimens set out in Table 2, 3, 4, 5 or 6.

In some embodiments, the method of treating a disorder associated with BCMA expression comprises administering one or more additional therapeutic agents to the patient. In preferred embodiments, a corticosteroid is administered, preferably dexamethasone. In some embodiments, the corticosteroid (e.g. dexamethasone) is administered to the patient weekly for at least the first treatment cycle, at least the first two treatment cycles, or at least the first three treatment cycles. In preferred embodiments, the corticosteroid (e.g. dexamethasone) is administered to the patient (e.g. weekly) for three treatment cycles. In some embodiments, the BCMA therapeutic agent (e.g. 42-TCBcv), cytokine inhibitor (e.g. IL-6 inhibitor) and corticosteroid (e.g. dexamethasone) are administered to the patient in accordance with the regimen set out in Table 7, 8 or 9.

### B) Lead With Therapeutic Agent

In some embodiments, the cytokine inhibitor (e.g. IL-6 inhibitor) is administered to the subject as one or more doses, wherein the first dose of the cytokine inhibitor (e.g. IL-6 inhibitor) is administered after administration of a first dose of the therapeutic agent (e.g. T cell engager). Without being bound by theory, administration of the cytokine inhibitor (e.g. IL-6 inhibitor) after the therapeutic agent (e.g. T cell engager) may prevent or reduce the development of CRS or treat CRS associated with administration of the therapeutic agent (e.g. T cell engager). In addition, in embodiments in which the therapeutic agent is a T cell engager (e.g. multispecific T-cell engaging antibody, CAR or CAR T cell), administration of the cytokine inhibitor (e.g. IL-6 inhibitor) described herein may increase efficacy of the therapeutic agent e.g. by potentiating T cell activation.

In some embodiments, the first dose of the cytokine inhibitor (e.g. IL-6 inhibitor) is administered to the subject 1-28 days after, e.g. 1, 2, 3, 4, 5, 6, 7, 8, 9, 10, 11, 12, 13, 14, 15, 16, 17, 18, 19, 20, 21, 22, 23, 24, 25, 26, 27 or 28 days after, or on the same day but after, the first dose of the therapeutic agent (e.g. T cell engager). In preferred embodiments, the first dose of the cytokine inhibitor (e.g. IL-6 inhibitor) is administered to the subject 7 days after the first dose of the therapeutic agent (e.g. T cell engager).

In some embodiments, two or more doses (e.g. two, three, four, five, six or seven doses) of the cytokine inhibitor (e.g. IL-6 inhibitor) are administered to the subject 1-28 days after, e.g. 1, 2, 3, 4, 5, 6, 7, 8, 9, 10, 11, 12, 13, 14, 15, 16, 17, 18, 19, 20, 21, 22, 23, 24, 25, 26, 27 or 28 days after, or on the same day but after, preferably 7 days after, the first dose of the therapeutic agent (e.g. T cell engager). In some embodiments, a first dose of the cytokine inhibitor (e.g. IL-6 inhibitor) is administered to the subject 7 days after the first dose of the therapeutic agent (e.g. T cell engager), and one or more further doses of the cytokine inhibitor (e.g. IL-6 inhibitor) is administered to the subject after (e.g. 8-27 days after) administration of the first dose of the therapeutic agent (e.g. T cell engager).

In some embodiments of any aspect of the invention, the cytokine inhibitor (e.g. IL-6 inhibitor) is administered to the patient as a treatment comprising at least one treatment cycle. In some embodiments, the treatment comprises a first treatment cycle wherein the cytokine inhibitor (e.g. IL-6 inhibitor) is administered to the patient on days 8-28 and the therapeutic agent (e.g. T cell engager) is administered to the patient on days 1, 4, 8, 15 and 22. In alternative embodiments, the treatment comprises a first treatment cycle wherein the cytokine inhibitor (e.g. IL-6 inhibitor) is administered to the patient on days 8-14 and days 22-28 and the therapeutic agent (e.g. T cell engager) is administered to the patient on days 1, 4, 8, 15 and 22. In some embodiments, the treatment comprises a second treatment cycle, wherein the cytokine inhibitor (e.g. IL-6 inhibitor) is administered to the patient on days 8-28 and the therapeutic agent (e.g. T cell engager) is administered to the patient on days 1, 8, 15 and 22. In alternative embodiments, the treatment comprises a second treatment cycle, wherein the cytokine inhibitor (e.g. IL-6 inhibitor) is administered to the patient on days 8-14 and days 22-28 and the therapeutic agent (e.g. T cell engager) is administered to the patient on days 1, 8, 15 and 22. In some embodiments, the treatment comprises a third treatment cycle, optionally a fourth, fifth and sixth treatment cycle, wherein cytokine inhibitor (e.g. IL-6 inhibitor) is administered to the patient on days 8-28 and the therapeutic agent (e.g. T cell engager) is administered to the patient on days 1, 8, 15 and 22. In some embodiments, the patient continues to receive treatment (e.g. for the rest of their lives).

In some embodiments, the T cell engager is a BCMA therapeutic agent (e.g. 42-TCBcv). The BCMA therapeutic agent (e.g. 42-TCBcv) may be administered to the patient (e.g. multiple myeloma patient) in accordance with the regimen set out in Table 10 or Table 11.

TABLE 10

| | | |
|---|---|---|
| | Cycle 1 | Cycle 2+ |
| BCMA therapeutic agent (e.g. 42-TCBcv) | Days 1, 4, 8, 15, 22 | Days 1, 8, 15, 22 |
| Cytokine inhibitor (e.g. Compound 1 or iberdomide) | Days 8-28 | Days 8-28 |

TABLE 11

| | | |
|---|---|---|
| | Cycle 1 | Cycle 2+ |
| BCMA therapeutic agent (e.g. 42-TCBcv) | Days 1, 4, 8, 15, 22 | Days 1, 8, 15, 22 |
| Cytokine inhibitor (e.g. Compound 1 or iberdomide) | Days 8-14, 22-28 | Days 8-28 |

In some embodiments, the cytokine inhibitor (e.g. iberdomide or Compound 1) is administered orally. In some embodiments, the one or more doses of cytokine inhibitor (e.g. iberdomide or Compound 1) is administered at a fixed dose. In some embodiments, iberdomide is administered at a fixed dose of between about 0.03 mg to about 6 mg, between about 0.1 mg to about 4 mg, between about 0.3 mg to about 2 mg, or between about 1.0 mg to about 1.6 mg, e.g. about 1.0 mg, about 1.3 mg or about 1.6 mg. In some embodiments, Compound 1 is administered at a fixed dose of between about 0.05 mg to about 5 mg, between about 0.1 mg to about 2 mg, between about 0.2 mg to about 1.6 mg, or between about 0.3 mg to about 1.0 mg, e.g. about 0.3 mg, about 0.6 mg or about 1.0 mg.

In some embodiments, the BCMA therapeutic agent is administered intravenously. In preferred embodiments, the first dose of the BCMA therapeutic agent (e.g. 42-TCBcv) is administered subcutaneously. In some embodiments, the BCMA therapeutic agent (e.g. 42-TCBcv) is administered subcutaneously in the first cycle, optionally in the first and subsequent cycles. In some embodiments, the BCMA therapeutic agent (e.g. 42-TCBcv) is administered at a dose of between about 1 mg to about 100 mg, between about 1 mg to about 75 mg, between about 1 mg to about 50 mg, between about 1 mg to about 25 mg, or between about 1 mg to about 12 mg.

In some embodiments in which the cytokine inhibitor is Compound 1 or iberdomide, the BCMA therapeutic agent (e.g. 42-TCBcv) and the cytokine inhibitor may be administered to the patient (e.g. multiple myeloma patient) in accordance with the regimen set out in Table 12, 13 or 14.

TABLE 12

| | | |
|---|---|---|
| | Cycle 1 | Cycle 2+ |
| BCMA therapeutic agent (e.g. 42-TCBcv at a dose of about 1.0 mg to about 100 mg) | Days 1, 4, 8, 15, 22 | Days 1, 8, 15, 22 |
| Compound 1 at a fixed dose of about 0.3 mg, about 0.6 mg or about 1.0 mg | Days 8-28 | Days 8-28 |

TABLE 13

| | | |
|---|---|---|
| | Cycle 1 | Cycle 2+ |
| BCMA therapeutic agent (e.g. 42-TCBcv at a dose of about 1.0 mg to about 100 mg) | Days 1, 4, 8, 15, 22 | Days 1, 8, 15, 22 |
| Iberdomide at a fixed dose of about 1.0 mg, about 1.3 mg or about 1.6 mg | Days 8-28 | Days 8-28 |

TABLE 14

| | | |
|---|---|---|
| | Cycle 1 | Cycle 2+ |
| BCMA therapeutic agent (e.g. 42-TCBcv at a dose of about 1.0 mg to about 100 mg) | Days 1, 4, 8, 15, 22 | Days 1, 8, 15, 22 |
| Compound 1 at a fixed dose of about 0.6 mg or iberdomide at a fixed dose of about 1. 3 mg | Days 8-14, 12-28 | Days 8-28 |

In some embodiments, one or more additional therapeutic agents is administered to the patient. In preferred embodiments, a corticosteroid is administered, preferably dexamethasone. In some embodiments, the corticosteroid (e.g. dexamethasone) is administered to the patient weekly for at least the first treatment cycle, at least the first two treatment cycles, or at least the first three treatment cycles. In preferred embodiments, the corticosteroid (e.g. dexamethasone) is administered to the patient (e.g. weekly) for three treatment cycles. The dose of the corticosteroid (e.g. dexamethasone) may be about 40 mg per week (e.g. for patients up to and including 75 years old and not underweight) or about 20 mg per week (e.g. for patients older than 75 years or underweight, body mass index [BMI] <18.5). Preferably, the corticosteroid (e.g. dexamethasone) is administered orally or intravenously. In some embodiments, the corticosteroid (e.g. dexamethasone) is administered orally with the cytokine inhibitor (e.g. iberdomide or Compound 1). In some embodiments, the corticosteroid (e.g. dexamethasone) is administered intravenously on a day on which the BCMA therapeutic agent (e.g. 42-TCBcv) is administered.

In some embodiments, the BCMA therapeutic agent (e.g. 42-TCBcv), cytokine inhibitor and corticosteroid (e.g. dexamethasone) are administered to the patient (e.g. multiple myeloma patient) in accordance with the regimen set out in Table 15, 16 or 17.

TABLE 15

| | | |
|---|---|---|
| | Cycle 1 | Cycle 2+ |
| BCMA therapeutic agent (e.g. 42-TCBcv at a dose of about 1.0 mg to about 100 mg) | Days 1, 4, 8, 15, 22 | Days 1, 8, 15, 22 |
| Compound 1 at a fixed dose of about 0.3 mg, about 0.6 mg or about 1.0 mg | Days 8-28 | Days 8-28 |
| Corticosteroid (e.g. dexamethasone at about 40 mg / week for patients ≤ 75 and at about 20 mg / week for patients > 75 or underweight) | Once per week | Once per week up to the end of cycle 3 |

TABLE 16

| | | |
|---|---|---|
| | Cycle 1 | Cycle 2+ |
| BCMA therapeutic agent (e.g. 42-TCBcv at a dose of about 1.0 mg to about 100 mg) | Days 1, 4, 8, 1 5, 22 | Days 1, 8, 15, 22 |
| Iberdomide at a fixed dose of about 1.0 mg, about 1.3 mg or about 1.6 mg | Days 8-28 | Days 8-28 |
| Corticosteroid (e.g. dexamethasone at about 40 mg / week for patients ≤ 75 and at about 20 mg / week for patients > 75 or underweight) | Once per week | Once per week up to the end of cycle 3 |

TABLE 17

| | | |
|---|---|---|
| | Cycle 1 | Cycle 2+ |
| BCMA therapeutic agent (e.g. 42-TCBcv at a dose of about 1.0 mg to about 100 mg) | Days 1, 4, 8, 15, 22 | Days 1, 8, 15, 22 |
| Compound 1 at a fixed dose of about 0.6 mg or iberdomide at a fixed dose of about 1.3 mg | Days 8-14, 12-28 | Days 8-28 |
| Corticosteroid (e.g. dexamethasone at 40 mg / week for patients ≤ 75 and at 20 mg / week for patients > 75 or underweight) | Once per week | Once per week up to the end of cycle 3 |

In an aspect, there is provided a method of treating a disorder associated with BCMA expression, wherein the method comprises administering a BCMA therapeutic agent (e.g. 42-TCBcv) and a cytokine inhibitor (e.g. IL-6 inhibitor) to a subject, wherein the BCMA therapeutic agent and the cytokine inhibitor (e.g. IL-6 inhibitor) are administered to the subject in accordance with any one of the regimens set out in Table 10, 11, 12, 13 or 14.

In some embodiments, the method of treating a disorder associated with BCMA expression comprises administering one or more additional therapeutic agents to the patient. In preferred embodiments, a corticosteroid is administered, preferably dexamethasone. In some embodiments, the corticosteroid (e.g. dexamethasone) is administered to the patient weekly for at least the first treatment cycle, at least the first two treatment cycles, or at least the first three treatment cycles. In preferred embodiments, the corticosteroid (e.g. dexamethasone) is administered to the patient (e.g. weekly) for three treatment cycles. In some embodiments, the BCMA therapeutic agent (e.g. 42-TCBcv), cytokine inhibitor (e.g. IL-6 inhibitor) and corticosteroid (e.g. dexamethasone) are administered to the patient in accordance with the regimen set out in Table 15, 16 or 17.

### BCMA Binding Sequences

In some embodiments, the BCMA therapeutic agent (e.g. the anti-BCMA antibody, or antigen binding fragment thereof) comprises a CDR1H, CDR2H, CDR3H, CDR1L, CDR2L, and CDR3L region combination selected from the group of:
a) CDR1H region of SEQ ID NO:21, CDR2H region of SEQ ID NO:22, CDR3H region of SEQ ID NO:17, CDR1L region of SEQ ID NO:23, CDR2L region of SEQ ID NO:24, and CDR3L region of SEQ ID NO:20;
b) CDR1H region of SEQ ID NO:21, CDR2H region of SEQ ID NO:22, CDR3H region of SEQ ID NO:17, CDR1L region of SEQ ID NO:25, CDR2L region of SEQ ID NO:26, and CDR3L region of SEQ ID NO:20;
c) CDR1H region of SEQ ID NO:21, CDR2H region of SEQ ID NO:22, CDR3H region of SEQ ID NO:17, CDR1L region of SEQ ID NO:27, CDR2L region of SEQ ID NO:28, and CDR3L region of SEQ ID NO:20;
d) CDR1H region of SEQ ID NO:29, CDR2H region of SEQ ID NO:30, CDR3H region of SEQ ID NO:17, CDR1L region of SEQ ID NO:31, CDR2L region of SEQ ID NO:32, and CDR3L region of SEQ ID NO:33;
e) CDR1H region of SEQ ID NO:34, CDR2H region of SEQ ID NO:35, CDR3H region of SEQ ID NO:17, CDR1L region of SEQ ID NO:31, CDR2L region of SEQ ID NO:32, and CDR3L region of SEQ ID NO:33;
f) CDR1H region of SEQ ID NO:36, CDR2H region of SEQ ID NO:37, CDR3H region of SEQ ID NO:17, CDR1L region of SEQ ID NO:31, CDR2L region of SEQ ID NO:32, and CDR3L region of SEQ ID NO:33; and
g) CDR1H region of SEQ ID NO: 15, CDR2H region of SEQ ID NO:16, CDR3H region of SEQ ID NO:17, CDR1L region of SEQ ID NO:18, CDR2L region of SEQ ID NO:19, and CDR3L region of SEQ ID NO:20.

In preferred embodiments, the BCMA therapeutic agent (e.g. the anti-BCMA antibody, or antigen binding fragment thereof) comprises a CDR1H, CDR2H, CDR3H, CDR1L, CDR2L and CDR3L region combination selected from:
a) CDR1H region of SEQ ID NO:21, CDR2H region of SEQ ID NO:22, CDR3H region of SEQ ID NO:17, CDR1L region of SEQ ID NO:27, CDR2L region of SEQ ID NO:28, and CDR3L region of SEQ ID NO:20;
b) CDR1H region of SEQ ID NO:21, CDR2H region of SEQ ID NO:22, CDR3H region of SEQ ID NO: 17, CDR1L region of SEQ ID NO:25, CDR2L region of SEQ ID NO:26 , and CDR3L region of SEQ ID NO:20; or
c) CDR1H region of SEQ ID NO: 15, CDR2H region of SEQ ID NO: 16, CDR3H region of SEQ ID NO:17, CDR1L region of SEQ ID NO:18, CDR2L region of SEQ ID NO:19, and CDR3L region of SEQ ID NO:20.

In any of the embodiments disclosed herein, a CDR1L region of SEQ ID NO:27 may be replaced with a CDR1L region of SEQ ID NO:62; and a CDR2L region of SEQ ID NO:28 may be replaced with a CDR2L region of SEQ ID NO:63. Accordingly, in some embodiments the multispecific (e.g. bispecific) antibody may comprise an anti-BCMA antibody, or antigen binding fragment thereof, comprising CDR1H region of SEQ ID NO:21, CDR2H region of SEQ ID NO:22, CDR3H region of SEQ ID NO:17, CDR1L region of SEQ ID NO:62, CDR2L region of SEQ ID NO:63, and CDR3L region of SEQ ID NO:20.

In any of the embodiments disclosed herein, a CDR1L region of SEQ ID NO:25 may be replaced with a CDR1L region of SEQ ID NO:60; and a CDR2L region of SEQ ID NO:26 may be replaced with a CDR2L region of SEQ ID NO:61. Accordingly, in some embodiments the multispecific (e.g. bispecific) antibody may comprise an anti-BCMA antibody, or antigen binding fragment thereof, comprising CDR1H region of SEQ ID NO:21, CDR2H region of SEQ ID NO:22, CDR3H region of SEQ ID NO:17, CDR1L region of SEQ ID NO:60, CDR2L region of SEQ ID NO:61 , and CDR3L region of SEQ ID NO:20.

In any of the embodiments disclosed herein, a CDR1L region of SEQ ID NO: 18 may be replaced with a CDR1L region of SEQ ID NO:58, and a CDR2L region of SEQ ID NO:19 may be replaced with a CDR2L region of SEQ ID NO: 59. Accordingly, in some embodiments the multispecific (e.g. bispecific) antibody may comprise an anti-BCMA antibody, or antigen binding fragment thereof, comprising CDR1H region of SEQ ID NO:15, CDR2H region of SEQ ID NO:16, CDR3H region of SEQ ID NO:17, CDR1L region of SEQ ID NO:58, CDR2L region of SEQ ID NO:59, and CDR3L region of SEQ ID NO:20.

In particularly preferred embodiments, the BCMA therapeutic agent (e.g. the anti-BCMA antibody, or antigen binding fragment thereof) comprises a VH region comprising a CDR1H region of SEQ ID NO:21, a CDR2H region of SEQ ID NO:22 and a CDR3H region of SEQ ID NO: 17 and a VL region comprising a CDR1L region of SEQ ID NO:27, a CDR2L region of SEQ ID NO:28 and a CDR3L region of SEQ ID NO:20.

In some embodiments, the BCMA therapeutic agent (e.g. the anti-BCMA antibody, or antigen binding fragment thereof) comprises a VH and a VL selected from the group consisting of:
a) a VH region of SEQ ID NO:10 and a VL region of SEQ ID NO:12,
b) a VH region of SEQ ID NO:10 and a VL region of SEQ ID NO:13,
c) a VH region of SEQ ID NO:10 and a VL region of SEQ ID NO:14,
d) a VH region of SEQ ID NO:38 and a VL region of SEQ ID NO:12,
e) a VH region of SEQ ID NO:39 and a VL region of SEQ ID NO:12,
f) a VH region of SEQ ID NO:40 and a VL region of SEQ ID NO:12, or
g) a VH region of SEQ ID NO: 9 and a VL region of SEQ ID NO:11.

In some embodiments, the BCMA therapeutic agent (e.g. the anti-BCMA antibody, or antigen binding fragment thereof) comprises a VH and a VL selected from the group consisting of:
a) a VH comprising an amino acid sequence that is at least 75% identical, at least 90% identical, at least 95% identical, or identical to the amino acid sequence of SEQ ID NO:10 and a VL comprising an amino acid sequence that is at least 90% identical, at least 95% identical, or identical to the amino acid sequence of SEQ ID NO: 12;
b) a VH comprising an amino acid sequence that is at least 75% identical, at least 90% identical, at least 95% identical, or identical to the amino acid sequence of SEQ ID NO:10 and a VL comprising an amino acid sequence that is at least 90% identical, at least 95% identical, or identical to the amino acid sequence of SEQ ID NO:13;
c) a VH comprising an amino acid sequence that is at least 75% identical, at least 90% identical, at least 95% identical, or identical to the amino acid sequence of SEQ ID NO:10 and a VL comprising an amino acid sequence that is at least 90% identical, at least 95% identical, or identical to the amino acid sequence of SEQ ID NO:14;
d) a VH comprising an amino acid sequence that is at least 75% identical, at least 90% identical, at least 95% identical, or identical to the amino acid sequence of SEQ ID NO:38 and a VL comprising an amino acid sequence that is at least 90% identical, at least 95% identical, or identical to the amino acid sequence of SEQ ID NO:12;
e) a VH comprising an amino acid sequence that is at least 75% identical, at least 90% identical, at least 95% identical, or identical to the amino acid sequence of SEQ ID NO:39 and a VL comprising an amino acid sequence that is at least 90% identical, at least 95% identical, or identical to the amino acid sequence of SEQ ID NO:12;
f) a VH comprising an amino acid sequence that is at least 75% identical, at least 90% identical, at least 95% identical, or identical to the amino acid sequence of SEQ ID NO:40 and a VL comprising an amino acid sequence that is at least 90% identical, at least 95% identical, or identical to the amino acid sequence of SEQ ID NO:12; or
g) a VH comprising an amino acid sequence that is at least 75% identical, at least 90% identical, at least 95% identical to, or identical to the amino acid sequence of SEQ ID NO: 9 and a VL comprising an amino acid sequence that is at least 75% identical, at least 90% identical, at least 95% identical, or identical to the amino acid sequence of SEQ ID NO:11.

In some embodiments, the BCMA therapeutic agent (e.g. the anti-BCMA antibody, or antigen binding fragment thereof) comprises a VH and a VL selected from the group consisting of:
a) a VH region of SEQ ID NO:10 and a VL region of SEQ ID NO:13,
b) a VH region of SEQ ID NO:10 and a VL region of SEQ ID NO:14, or
c) a VH region of SEQ ID NO: 9 and a VL region of SEQ ID NO:11.

In particularly preferred embodiments, the BCMA therapeutic agent (e.g. the anti-BCMA antibody, or antigen binding fragment thereof) comprises a VH region of SEQ ID NO:10 and a VL region of SEQ ID NO:14.

In some embodiments, the BCMA therapeutic agent (e.g. the anti-BCMA antibody, or antigen binding fragment thereof), comprises the CDR3H, CDR3L, CDR1H, CDR2H, CDR1L, and CDR2L of one of GSK2857916, AMG-420, AMG-701, JNJ-957, JNJ-64007957, PF-06863135, REGN-5458, or TNB-383B. In some embodiments, the BCMA therapeutic agent (e.g. the anti-BCMA antibody, or antigen binding fragment thereof) comprises the VH and VL of one of GSK2857916, AMG-420, AMG-701, JNJ-957, JNJ-64007957, PF-06863135, REGN-5458, or TNB-383B. In some embodiments, the anti-BCMA antibody is BCMA tri-specific [Affirmed], AFM26 [Affirmed], Ab-957 [Janssen], or BCMA/PD-L1 [Immune pharmaceuticals].

In some embodiments, the BCMA therapeutic agent is a multispecific (e.g. bispecific) antibody of the invention which comprises an anti-BCMA antibody, or antigen binding fragment thereof, comprising a CDR1H, CDR2H, CDR3H, CDR1L, CDR2L, and CDR3L region combination selected from the group of:
a) CDR1H region of SEQ ID NO:21,CDR2H region of SEQ ID NO:22, CDR3H region of SEQ ID NO: 17, CDR1L region of SEQ ID NO:23, CDR2L region of SEQ ID NO:24, and CDR3L region of SEQ ID NO:20;
b) CDR1H region of SEQ ID NO:21. CDR2H region of SEQ ID NO:22, CDR3H region of SEQ ID NO: 17, CDR1L region of SEQ ID NO:25, CDR2L region of SEQ ID NO:26, and CDR3L region of SEQ ID NO:20;
c) CDR1H region of SEQ ID NO:21, CDR2H region of SEQ ID NO:22, CDR3H region of SEQ ID NO:17 CDR1L region of SEQ ID NO:27, CDR2L region of SEQ ID NO:28, and CDR3L region of SEQ ID NO:20;
d) CDR1H region of SEQ ID NO:29, CDR2H region of SEQ ID NO:30, CDR3H region of SEQ ID NO:17 CDR1L region of SEQ ID NO:31, CDR2L region of SEQ ID NO:32, and CDR3L region of SEQ ID NO:33;
e) CDR1H region of SEQ ID NO:34, CDR2H region of SEQ ID NO:35, CDR3H region of SEQ ID NO:17 CDR1L region of SEQ ID NO:31, CDR2L region of SEQ ID NO:32, and CDR3L region of SEQ ID NO:33;
f) CDR1H region of SEQ ID NO:36, CDR2H region of SEQ ID NO:37, CDR3H region of SEQ ID NO:17 CDR1L region of SEQ ID NO:31, CDR2L region of SEQ ID NO:32, and CDR3L region of SEQ ID NO:33; or
g) CDR1H region of SEQ ID NO: 15, CDR2H region of SEQ ID NO:16, CDR3H region of SEQ ID NO:17, CDR1L region of SEQ ID NO:18, CDR2L region of SEQ ID NO:19, and CDR3L region of SEQ ID NO:20,
and an anti-CD3 antibody, or antigen binding fragment thereof, comprising a CDR1H region of SEQ ID NO:1, a CDR2H region of SEQ ID NO:2, a CDR3H region of SEQ ID NO:3, a CDR1L region of SEQ ID NO:4, a CDR2L region of SEQ ID NO:5 and a CDR3L region of SEQ ID NO: 6.

In particularly preferred embodiments, the multispecific (e.g. bispecific) antibody of the invention comprises:
a) an anti-BCMA antibody, or antigen binding fragment thereof, comprising a VH region comprising a CDR1H region of SEQ ID NO:21, a CDR2H region of SEQ ID NO:22 and a CDR3H region of SEQ ID NO:17 and a VL region comprising a CDR1L region of SEQ ID NO:27, a CDR2L region of SEQ ID NO:28 and a CDR3L region of SEQ ID NO:20; and
b) an anti-CD3 antibody, or antigen binding fragment thereof, comprising a CDR1H region of SEQ ID NO:1, a CDR2H region of SEQ ID NO:2, a CDR3H region of SEQ ID NO:3, a CDR1L region of SEQ ID NO:4, a CDR2L region of SEQ ID NO:5 and a CDR3L region of SEQ ID NO:6.

In some embodiments, the multispecific (e.g. bispecific) antibody of the invention comprises an anti-BCMA antibody, or antigen binding fragment thereof, comprising a VH and a VL selected from the group consisting of:
a) a VH region of SEQ ID NO:10 and a VL region of SEQ ID NO:12,
b) a VH region of SEQ ID NO:10 and a VL region of SEQ ID NO:13,
c) a VH region of SEQ ID NO:10 and a VL region of SEQ ID NO:14,
d) a VH region of SEQ ID NO:38 and a VL region of SEQ ID NO:12,
e) a VH region of SEQ ID NO:39 and a VL region of SEQ ID NO:12,
f) a VH region of SEQ ID NO:40 and a VL region of SEQ ID NO:12, or
g) a VH region of SEQ ID NO: 9 and a VL region of SEQ ID NO:11, and
an anti-CD3 antibody, or antigen binding fragment thereof, comprising a VH region of SEQ ID NO:7 and a VL region of SEQ ID NO:8.

In particularly preferred embodiments, the multispecific (e.g. bispecific) antibody of the invention comprises an anti-BCMA antibody, or antigen binding fragment thereof, comprising a VH region of SEQ ID NO:10 and a VL region of SEQ ID NO:14, and an anti-CD3 antibody, or antigen binding fragment thereof, comprising a VH region of SEQ ID NO:7 and a VL region of SEQ ID NO:8.

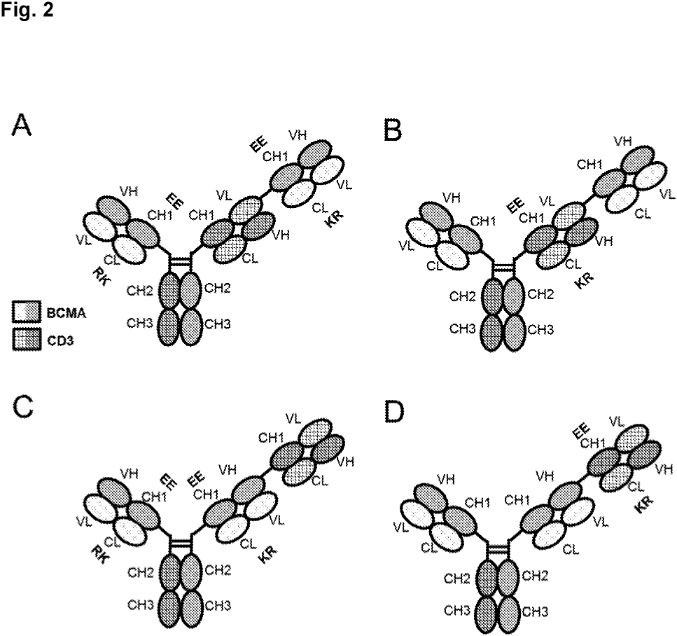

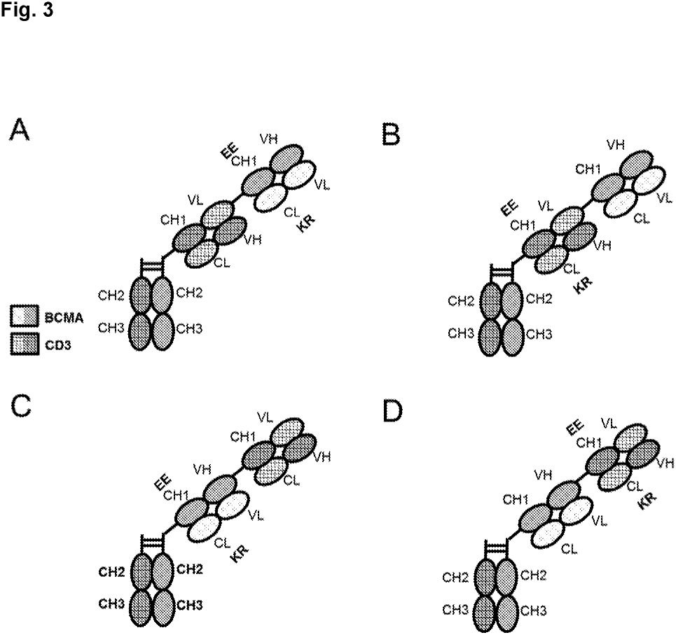

In preferred embodiments, the bispecific antibody of the invention comprises the following SEQ ID NOs (as mentioned in Tables 23A and 24B below):
83A10-TCBcv: 48, 45, 46, 47 (x2) (FIG. 2A)
22-TCBcv: 48, 52, 53, 54 (x2) (FIG. 2A)
42-TCBcv: 48, 55, 56, 57 (x2) (FIG. 2A)

The term “83A10-TCBcv” as used herein refers to a bispecific antibody specifically binding to BCMA and CD3 as specified by its heavy and light chain combination of SEQ ID NO:45, SEQ ID NO:46, SEQ ID NO:47 (2x), and SEQ ID NO:48, and as shown in FIG. 2A and described in EP14179705.

The term “22-TCBcv” as used herein refers to the bispecific antibody of Mab22 as specified by its heavy and light chain combination of SEQ ID NO:48, SEQ ID NO:52, SEQ ID NO:53, and SEQ ID NO:54 (2x), and as shown in FIG. 2A and described in WO 2017/021450.

The term “42-TCBcv” as used herein refers to the bispecific antibody of Mab42 as specified by its heavy and light chain combination of SEQ ID NO:48, SEQ ID NO:55, SEQ ID NO:56, and SEQ ID NO:57 (2x), and as shown in FIG. 2A and described in WO 2017/021450. In the present application, 42-TCBcv is referred to interchangeably as CC-93269.

In preferred embodiments, the bispecific antibody of the invention is 42-TCBcv.The present inventors have identified the cytokine inhibitors (e.g. IL-6 inhibitors) described herein may be administered to a subject at a dose sufficient to supress secretion of pro-inflammatory cytokines mediated by 42-TCBcv, e.g. secretion of pro-inflammatory cytokines from myeloid cells (e.g. macrophages and/or monocytes), wherein the dose of cytokine inhibitor (e.g. IL-6 inhibitor) does not reduce killing of BCMA-expressing cells (e.g. multiple myeloma cells) mediated by the 42-TCBcv. Thus, the cytokine inhibitors (e.g. IL-6 inhibitors) may prevent or reduce the development of CRS in a subject who has or will receive 42-TCBcv, without reducing killing of BCMA-expressing cells (e.g. multiple myeloma cells) mediated by the 42-TCBcv (see e.g. Example 9).

In some embodiments, the cytokine inhibitor (e.g. IL-6 inhibitor) increases killing of BCMA-expressing cells (e.g. multiple myeloma cells) mediated by 42-TCBcv. In vitro, 42-TCBcv mediated killing of BCMA-expressing cells (e.g. multiple myeloma cells) may be measured in a co-culture of healthy donor T cells and BCMA-expressing target cells (e.g. multiple myeloma cells) at a ratio of 5:1, T-cells to target cells. In vivo, tumour measurements can be used to assess 42-TCBcv mediated killing of multiple myeloma cells, for example in a H929 xenograft mouse model.

In some embodiments, the BCMA therapeutic agent is 42-TCBcv and the cytokine inhibitor (e.g. IL-6 inhibitor) is pomalidomide. In some embodiments, the BCMA therapeutic agent is 42-TCBcv and the cytokine inhibitor (e.g. IL-6 inhibitor) is iberdomide. In some embodiments, the BCMA therapeutic agent is 42-TCBcv and the cytokine inhibitor (e.g. IL-6 inhibitor) is lenalidomide. In some embodiments, the BCMA therapeutic agent is 42-TCBcv and the cytokine inhibitor (e.g. IL-6 inhibitor) is avadomide. In some embodiments, the BCMA therapeutic agent is 42-TCBcv and the cytokine inhibitor (e.g. IL-6 inhibitor) is Compound 1.

In one aspect, the present invention provides 42-TCBcv for use in a method of treating a disorder associated with BCMA expression in a subject wherein the method comprises
a) administering to the subject the 42-TCBcv; and
b) administering to the subject pomalidomide at a dose sufficient to prevent or reduce the development of CRS in the subject.

In one aspect, the present invention provides 42-TCBcv for use in a method of treating a disorder associated with BCMA expression in a subject wherein the method comprises
a) administering to the subject the 42-TCBcv; and
b) administering to the subject iberdomide at a dose sufficient to prevent or reduce the development of CRS in the subject.

In one aspect, the present invention provides 42-TCBcv for use in a method of treating a disorder associated with BCMA expression in a subject wherein the method comprises
a) administering to the subject the 42-TCBcv; and
b) administering to the subject lenalidomide at a dose sufficient to prevent or reduce the development of CRS in the subject.

In one aspect, the present invention provides 42-TCBcv for use in a method of treating a disorder associated with BCMA expression in a subject wherein the method comprises
a) administering to the subject the 42-TCBcv; and
b) administering to the subject avadomide at a dose sufficient to prevent or reduce the development of CRS in the subject.

In one aspect, the present invention provides 42-TCBcv for use in a method of treating a disorder associated with BCMA expression in a subject wherein the method comprises
a) administering to the subject the 42-TCBcv; and
b) administering to the subject Compound 1 at a dose sufficient to prevent or reduce the development of CRS in the subject.

Alternatively, the BCMA therapeutic agent may be a CAR directed to BCMA or a T cell expressing at least one CAR directed to BCMA.

In some embodiments, the BCMA therapeutic agent (e.g. BCMA CAR or BCMA CAR T cell) comprises the VH CDR1, CDR2 and CDR3, and the VL CDR1, CDR2 and CDR3 sequences set out in Table 18.

TABLE 18

CDR amino acid coordinates of CDR1, CDR2, and CDR3 of SEQ ID NOs: 76 through to 85

| | | | | |
|---|---|---|---|---|
| SEQ ID NO: | Description | CDR Amino Acid Coordinates | | |
| | | CDR1 | CDR2 | CDR3 |
| 76 | CD11D5.3 | 31-35 | 50-66 | 99-106 |
| | VH | | | |
| 77 | CD11D5.3 | 24-38 | 54-60 | 93-101 |
| | VL1 | | | |
| 78 | CD11D5.3 | 24-38 | 54-60 | 93-101 |
| | VL2 | | | |
| | (alternative) | | | |
| 79 | CD11D5.3 | 24-38 | 54-60 | 93-101 |
| | VL3 | | | |
| | (alternative) | | | |
| 80 | A7D12.2 | 31-35 | 50-66 | 99-111 |
| | VH | | | |
| 81 | A7D12.2 | 24-34 | 50-56 | 89-97 |
| | VL | | | |
| 82 | C12A32.3 | 31-35 | 50-66 | 99-106 |
| | VH | | | |
| 83 | C12A32.3 | 24-38 | 54-60 | 93-101 |
| | VL | | | |
| 84 | C13F12.1 | 31-35 | 50-66 | 99-106 |
| | VH | | | |
| 85 | C13F12.1 | 24-38 | 54-60 | 93-101 |
| | VL | | | |

In some embodiments, the BCMA therapeutic agent (e.g. BCMA CAR or BCMA CAR T cell) comprises a VH and VL, wherein:
a) the VH comprises the CDRs of SEQ ID NO:76 and the VL comprises the CDRs of SEQ ID NO:77 as set out in Table 18, optionally wherein the VH comprises SEQ ID NO:76 and the VL comprises SEQ ID NO:77;
b) the VH comprises the CDRs of SEQ ID NO:76 and the VL comprises the CDRs of SEQ ID NO:78 as set out in Table 18, optionally wherein the VH comprises SEQ ID NO:76 and the VL comprises SEQ ID NO:78;
c) the VH comprises the CDRs of SEQ ID NO:76 and the VL comprises the CDRs of SEQ ID NO:79 as set out in Table 18, optionally wherein the VH comprises SEQ ID NO:76 and the VL comprises SEQ ID NO:79;
d) the VH comprises the CDRs of SEQ ID NO:80 and the VL comprises the CDRs of SEQ ID NO:81 as set out in Table 18, optionally wherein the VH comprises SEQ ID NO:80 and the VL comprises SEQ ID NO:81;
e) the VH comprises the CDRs of SEQ ID NO:82 and the VL comprises the CDRs of SEQ ID NO:83 as set out in Table 18, optionally wherein the VH comprises SEQ ID NO:82 and the VL comprises SEQ ID NO:83; or
f) the VH comprises the CDRs of SEQ ID NO:84 and the VL comprises the CDRs of SEQ ID NO:85 as set out in Table 18, optionally wherein the VH comprises SEQ ID NO:84 and the VL comprises SEQ ID NO:85.

In some embodiments, the BCMA therapeutic agent (e.g. BCMA CAR or BCMA CAR T cell) comprises a VH and VL selected from the group consisting of:
a) a VH comprising an amino acid sequence that is at least 75% identical, at least 90% identical, at least 95% identical, or identical to the amino acid sequence of SEQ ID NO:76 and a VL comprising an amino acid sequence that is at least 90% identical, at least 95% identical, or identical to the amino acid sequence of SEQ ID NO:77;
b) a VH comprising an amino acid sequence that is at least 75% identical, at least 90% identical, at least 95% identical, or identical to the amino acid sequence of SEQ ID NO:76 and a VL comprising an amino acid sequence that is at least 90% identical, at least 95% identical, or identical to the amino acid sequence of SEQ ID NO:78;
c) a VH comprising an amino acid sequence that is at least 75% identical, at least 90% identical, at least 95% identical, or identical to the amino acid sequence of SEQ ID NO:76 and a VL comprising an amino acid sequence that is at least 90% identical, at least 95% identical, or identical to the amino acid sequence of SEQ ID NO:79;
d) a VH comprising an amino acid sequence that is at least 75% identical, at least 90% identical, at least 95% identical, or identical to the amino acid sequence of SEQ ID NO:80 and a VL comprising an amino acid sequence that is at least 90% identical, at least 95% identical, or identical to the amino acid sequence of SEQ ID NO:81;
e) a VH comprising an amino acid sequence that is at least 75% identical, at least 90% identical, at least 95% identical, or identical to the amino acid sequence of SEQ ID NO:82 and a VL comprising an amino acid sequence that is at least 90% identical, at least 95% identical, or identical to the amino acid sequence of SEQ ID NO:83; or
f) a VH comprising an amino acid sequence that is at least 75% identical, at least 90% identical, at least 95% identical, or identical to the amino acid sequence of SEQ ID NO:84 and a VL comprising an amino acid sequence that is at least 90% identical, at least 95% identical, or identical to the amino acid sequence of SEQ ID NO:85.

In preferred embodiments, the BCMA therapeutic agent (e.g. BCMA CAR or BCMA CAR T cell) comprises a VH comprising a CDR1H of SEQ ID NO:64, a CDR2H of SEQ ID NO:65 a CDR3H of SEQ ID NO:66, and a VL comprising a CDR1L, a CDR2L and a CDR3L set of sequences selected from:
a) CDR1L of SEQ ID NO:67, CDR2L of SEQ ID NO:68, and CDR3L of SEQ ID NO:69, optionally wherein the VH comprises SEQ ID NO:76 and the VL comprises SEQ ID NO:77;
b) CDR1L of SEQ ID NO:70, CDR2L of SEQ ID NO:71, and CDR3L of SEQ ID NO:72, optionally wherein the VH comprises SEQ ID NO:76 and the VL comprises SEQ ID NO:78; or
c) CDR1L of SEQ ID NO:73, CDR2L of SEQ ID NO:74, and CDR3L of SEQ ID NO:75 optionally wherein the VH comprises SEQ ID NO:76 and the VL comprises SEQ ID NO:79.

In certain embodiments, the BCMA CAR T cell is ide-cel, idecabtagene-vicleucel or bb21217. In some embodiments the CAR T cell is orva-cel or JCARH125.

In certain embodiments, the BCMA CAR T cell is KITE-585 (Kite Pharmaceuticals), P-BCMA-101 (Poseida Therapeutics), CART-BCMA (Novartis), LCAR-B38M (Legend Biotech), JNJ-528 (Janssen Biotech), P-BCMA-101 (Poseida Therapeutics), CT053 (CARsgen Therapeutics), CTX120 (CRISPR Therapeutics), ET140 (Juno Therapeutics), UCART-BCMA (Cellectis), P-BCMA-101 (Poseida), JNJ-528/LCAR-B38M (Johnson & Johnson) or BCMA CAR T cells from Radiance Bio or Second Affiliated Hospital of Henan University of traditional Chinese Medicine/Hrain Biotechnology Co. Ltd.).. In certain embodiments, the BCMA CAR T cell is MCARH171, FCARH143, CTX120, CT053 (First Affiliated Hospital of Wenzhou Medical University, CN), or BCMA-CART (Hrain Biotechnology, Shanghai CN).

### Multispecific Antibody Format

Formats for multispecific antibodies are known in the state of the art. For example, bispecific antibody formats are described in Kontermann RE (2012) MAbs. 4(2): 182-197; Holliger P and Hudson PJ (2005) Nat. Biotechnol. 23(9): 1126-1136; Chan AC and Carter PJ (2010) Nature Reviews Immunology. 10(5): 301-316 and Cuesta AM et al. (2010) Trends Biotechnol. 28(7): 355-362.

The multispecific (e.g. bispecific) antibodies of the invention may have any format. Multispecific and bispecific antibody formats include, for example, multivalent single chain antibodies, diabodies and triabodies, and antibodies having the constant domain structure of full length antibodies to which further antigen-binding domains (e.g., single chain Fv, a tandem scFv, a VH domain and/or a VL domain, Fab, or (Fab)₂,) are linked via one or more peptide-linkers, as well as antibody mimetics such as DARPins. In some embodiments, the multispecific (e.g. bispecific) antibodies of the invention have the format of an scFv such as a bispecific T cell engager (BITE^{®}). In some embodiments, the antibodies of the invention are single chain antibodies which comprise a first domain which binds to BCMA, a second domain which binds to a T cell antigen (e.g. CD3), and a third domain which comprises two polypeptide monomers, each comprising a hinge, a CH2 domain and a CH3 domain, wherein the two polypeptide monomers are fused to each other via a peptide linker (e.g. (hinge-CH2-CH3-linker-hinge-CH2-CH3)).

The “valency” of an antibody denotes the number of binding domains. As such, the terms “bivalent”, “trivalent”, and “multivalent” denote the presence of two binding domains, three binding domains, and multiple binding domains, respectively. The multispecific (e.g. bispecific) antibodies of the invention may have more than one binding domain capable of binding to each target antigen (i.e., the antibody is trivalent or multivalent). In preferred embodiments, the multispecific (e.g. bispecific) antibodies of the invention have more than one binding domain capable of binding to the same epitope of each target antigen. In some embodiments, the multispecific (e.g. bispecific) antibodies of the invention have more than one binding domain capable of binding to different epitopes on each target antigen.

The multispecific (e.g. bispecific) antibodies of the invention may be bivalent, trivalent or tetravalent. In preferred embodiments, the multispecific (e.g. bispecific) antibody is trivalent, preferably wherein the trivalent antibody is bivalent for BCMA. Thus, the bispecific antibody of the invention may be trivalent, wherein the trivalent antibody is bivalent for BCMA.

The multispecific (e.g. bispecific) antibodies can be full length from a single species, or can be chimerized or humanized. For an antibody with more than two antigen-binding domains, some binding domains may be identical, as long as the protein has binding domains for two different antigens.

The multispecific (e.g. bispecific) antibodies of the invention can have a bispecific heterodimeric format. In some embodiments, the bispecific antibody comprises two different heavy chains and two different light chains. In other embodiments, the multispecific (e.g. bispecific) antibody comprises two identical light chains and two different heavy chains. In some embodiments, in the multispecific (e.g. bispecific) antibodies of the invention one of the two pairs of heavy chain and light chain (HC/LC) specifically binds to CD3 and the other one specifically binds to BCMA.

In embodiments in which the bispecific antibodies of the invention are bivalent, they may comprise one anti-BCMA antibody and one anti-CD3 antibody (referred to herein as the “1+1” format).

In embodiments in which the BCMA and CD3 antibodies are Fabs, the bivalent bispecific antibodies in the 1+1 format may have the format: CD3 Fab – BCMA Fab (i.e. when no Fc is present). Alternatively, the bispecific antibodies may have the format: Fc – CD3 Fab – BCMA Fab; Fc- BCMA Fab – CD3 Fab; or BCMA Fab – Fc – CD3 Fab (i.e. when an Fc is present). In preferred embodiments, the bivalent bispecific antibodies have the format BCMA Fab – Fc – CD3 Fab.

“CD3 Fab - BCMA Fab” means that the CD3 Fab is bound via its N-terminus to the C-terminus of the BCMA Fab.

“Fc - BCMA Fab – CD3 Fab” means that the BCMA Fab is bound via its C-terminus to the N-terminus of the Fc, and the CD3 Fab is bound via its C-terminus to the N-terminus of the BCMA Fab.

“Fc – CD3 Fab – BCMA Fab” means that the CD3 Fab is bound via its C-terminus to the N-terminus of the Fc, and the BCMA Fab is bound via its C-terminus to the N-terminus of the CD3 Fab.

“BCMA Fab – Fc – CD3 Fab” means that the BCMA and CD3 Fab fragments are bound via their C-terminus to the N-terminus of the Fc.

In embodiments in which the bispecific antibodies of the invention are trivalent, they may comprise two anti-BCMA antibodies and one anti-CD3 antibody (referred to herein as the “2+1” format).

In embodiments in which the BCMA and CD3 antibodies are Fabs, the trivalent bispecific antibodies in the 2+1 format may have the format: CD3 Fab – BCMA Fab – BCMA Fab; or BCMA Fab – CD3 Fab – BCMA Fab (i.e. when no Fc is present). Alternatively, the bispecific antibodies may have the format: BCMA Fab – Fc – CD3 Fab – BCMA Fab; BCMA Fab – Fc –BCMA Fab – CD3 Fab; or CD3 Fab – Fc – BCMA Fab – BCMA Fab (i.e. when an Fc is present). In preferred embodiments, the trivalent bispecific antibodies have the format BCMA Fab – Fc –CD3 Fab – BCMA Fab.

“CD3 Fab – BCMA Fab – BCMA Fab” means that the CD3 Fab is bound via its C-terminus to the N-terminus of the first BCMA Fab, and the first BCMA Fab is bound via its C-terminus to the N-terminus of the second BCMA Fab.

“BCMA Fab – CD3 Fab – BCMA Fab” means that the first BCMA Fab is bound via its C-terminus to the N-terminus of the CD3 Fab, and the CD3 Fab is bound via its C-terminus to the N-terminus of the second BCMA Fab.

“BCMA Fab – Fc – CD3 Fab – BCMA Fab” means that the first BCMA Fab and the CD3 Fab are bound via their C-terminus to the N-terminus of the Fc, and the second BCMA Fab is bound via its C-terminus to the N-terminus of the CD3 Fab.

“BCMA Fab – Fc – BCMA Fab – CD3 Fab” means that the first BCMA Fab and the second BCMA Fab are bound via their C-terminus to the N-terminus of the Fc, and the CD3 Fab is bound via its C-terminus to the N-terminus of the second BCMA Fab.

“CD3 Fab – Fc – BCMA Fab – BCMA Fab” means that the CD3 Fab and the first BCMA Fab are bound via their C-terminus to the N-terminus of the Fc, and the second BCMA Fab is bound via its C-terminus to the N-terminus of the first BCMA Fab.

In some embodiments, the bispecific antibodies of the invention may comprise not more than one BCMA Fab specifically binding to BCMA, and not more than one CD3 Fab specifically binding to CD3 and not more than one Fc part.

In some embodiments, the bispecific antibody comprises not more than one CD3 Fab specifically binding to CD3, not more than two BCMA Fabs specifically binding to BCMA and not more than one Fc part. In some embodiments, not more than one CD3 Fab and not more than one BCMA Fab are linked to the Fc part and linking is performed via C-terminal binding of the Fab(s) to the hinge region of the Fc part. In some embodiments, the second BCMA Fab is linked via its C-terminus either to the N-terminus of the CD3 Fab or to the hinge region of the Fc part and is therefore between the Fc part of the bispecific antibody and the CD3 Fab.

In embodiments comprising two BCMA Fabs, the BCMA Fabs are preferably derived from the same antibody and are preferably identical in the CDR sequences, variable domain sequences VH and VL and/or the constant domain sequences CH1 and CL. Preferably, the amino acid sequences of the two BCMA Fab are identical.

The bispecific antibodies of the invention can also comprise scFvs instead of the Fabs. Thus, in some embodiments, the bispecific antibodies have any one of the above formats, wherein each Fab is replaced with a corresponding scFv.

The terms “Fab fragment” and “Fab” are used interchangeably herein and contain a single light chain (i.e. a constant domain CL and a VL) and a single heavy chain (i.e. the constant domain CH1 and a VH). The heavy chain of a Fab fragment is not capable of forming a disulfide bond with another heavy chain.

A “Fab′ fragment” contains a single light chain and a single heavy chain but in addition to the CH1 and the VH, a “Fab′ fragment” contains the region of the heavy chain between the CH1 and CH2 domains that is required for the formation of an inter-chain disulfide bond. Thus, two “Fab′ fragments” can associate via the formation of a disulphide bond to form a F(ab′)2 molecule.

A “F(ab′)2 fragment” contains two light chains and two heavy chains. Each chain includes a portion of the constant region necessary for the formation of an inter-chain disulfide bond between two heavy chains.

An “Fv fragment” contains only the variable regions of the heavy and light chain. It contains no constant regions.

A “single-domain antibody” is an antibody fragment containing a single antibody domain unit (e.g., VH or VL).

A “single-chain Fv” (“scFv”) is antibody fragment containing the VH and VL domain of an antibody, linked together to form a single chain. A polypeptide linker is commonly used to connect the VH and VL domains of the scFv.

A “tandem scFv”, also known as a TandAb^{®}, is a single-chain Fv molecule formed by covalent bonding of two scFvs in a tandem orientation with a flexible peptide linker.

A “bi-specific T cell engager” (BiTE^{®}) is a fusion protein consisting of two single-chain variable fragments (scFvs) on a single peptide chain. One of the scFvs binds to T cells via the CD3 receptor, and the other to a tumor cell antigen.

A “diabody” is a small bivalent and bispecific antibody fragment comprising a heavy (VH) chain variable domain connected to a light chain variable domain (VL) on the same polypeptide chain (VH-VL) connected by a peptide linker that is too short to allow pairing between the two domains on the same chain (Kipriyanov, Int. J. Cancer 77 (1998), 763-772). This forces pairing with the complementary domains of another chain and promotes the assembly of a dimeric molecule with two functional antigen binding sites.

A “DARPin” is a bispecific ankyrin repeat molecule. DARPins are derived from natural ankyrin proteins, which can be found in the human genome and are one of the most abundant types of binding proteins. A DARPin library module is defined by natural ankyrin repeat protein sequences, using 229 ankyrin repeats for the initial design and another 2200 for subsequent refinement. The modules serve as building blocks for the DARPin libraries. The library modules resemble human genome sequences. A DARPin is composed of 4 to 6 modules. Because each module is approx. 3.5 kDa, the size of an average DARPin is 16-21 kDa. Selection of binders is done by ribosome display, which is completely cell-free and is described in He M. and Taussig MJ., Biochem Soc Trans. 2007, Nov;35(Pt 5):962-5.

The components, e.g. the Fab fragments, of the bispecific antibodies of the invention may be chemically linked together by the use of an appropriate linker according to the state of the art. In preferred embodiments, a (Gly4-Ser1)₂ linker is used (Desplancq DK et al. (1994) Protein Eng. 7(8):1027-33; Mack M et al (1995) PNAS. 92(15): 7021-7025). “Chemically linked” (or “linked”) as used herein means that the components are linked by covalent binding. As the linker is a peptidic linker, such covalent binding is usually performed by biochemical recombinant means. For example, the binding may be performed using a nucleic acid encoding the VL and/or VH domains of the respective Fab fragments, the linker and the Fc part chain if the antibody comprises an Fc.

In the event that a linker is used, this linker may be of a length and sequence sufficient to ensure that each of the first and second domains can, independently from each other, retain their differential binding specificities.

### Fc

The antibodies (e.g. bispecific antibodies) of the invention may have an Fc or may not have an Fc. In preferred embodiments, the antibodies (e.g. bispecific antibodies) of the invention comprise an Fc, preferably a human Fc.

In certain embodiments, the Fc is a variant Fc, e.g. an Fc sequence that has been modified (for example by amino acid substitution, deletion and/or insertion) relative to a parent Fc sequence (for example an unmodified Fc polypeptide that is subsequently modified to generate a variant), to provide desirable structural features and/or biological activity,

Accordingly, the antibodies (e.g. bispecific antibodies) of the invention may comprise an Fc comprising one or more modifications, typically to alter one or more functional properties of the antibody, such as serum half-life, complement fixation, Fc receptor binding, and/or antigen-dependent cellular cytotoxicity. The Fc may be linked to the anti-BCMA and/or anti-CD3 Fab fragments in the multispecific (e.g. bispecific) antibodies of the invention.

The presence of an Fc has the advantage of extending the elimination half-life of the antibody. The antibodies (e.g. bispecific antibodies) of the invention may have an elimination half-life in mice or cynomolgus monkeys, preferably cynomolgus monkeys, of longer than 12 hours, preferably 3 days or longer. In some embodiments, the antibodies (e.g. bispecific antibodies) of the invention have an elimination half-life of about 1 to 12 days, which allows at least once or twice/week administration.

### Reduced Effector Function

Preferably, the multispecific (e.g. bispecific) antibodies of the invention comprise an Fc region (e.g. of IgG1 subclass) that comprises modifications to avoid FcR and Clq binding and minimize ADCC/CDC. This provides the advantage that the bispecific antibody mediates its tumor cell killing efficacy purely by the powerful mechanism of effector cell, e.g. T cell, redirection/activation. Therefore, additional mechanisms of action, such as effects on the complement system and on effector cells expressing FcR, are avoided and the risk of side-effects, such as infusion-related reactions, is decreased.

In preferred embodiments, the multispecific (e.g. bispecific) antibodies of the invention comprise an IgG, particularly IgGl, Fc region comprising the modifications L234A, L235A and P329G (numbered according to EU numbering).

### Heterodimerization

The multispecific (e.g. bispecific) antibodies of the invention may be heteromultimeric antibodies. Such heteromultimeric antibodies may comprise modifications in regions involved in interactions between antibody chains to promote correct assembly of the antibodies.

For example, the multispecific (e.g. bispecific) antibodies of the invention may comprise an Fc having one or more modification(s) in the CH2 and CH3 domain to enforce Fc heterodimerization. Alternatively or in addition, the multispecific (e.g. bispecific) antibodies of the invention may comprise modifications in the CH1 and CL region to promote preferential pairing between the heavy chain and light chain of a Fab fragment.

A number of strategies exist for promoting heterodimerization. These strategies may include the introduction of asymmetric complementary modifications into each of two antibody chains, such that both chains are compatible with each other and thus able to form a heterodimer, but each chain is not able to dimerize with itself. Such modifications may encompass insertions, deletions, conservative and non-conservative substitutions and rearrangements.

Heterodimerization may be promoted by the introduction of charged residues to create favourable electrostatic interactions between a first antibody chain and a second antibody chain. For example, one or more positively charged amino acids amino acid may be introduced into a first antibody chain, and one or more negatively charged amino acids may be introduced into a corresponding positions in a second antibody chain

Alternatively or in addition, heterodimerization may be promoted by the introduction of steric hindrance between contacting residues. For example, one or more residues with a bulky side chain may be introduced into a first antibody chain, and a one or more residues able to accommodate the bulky side chain may be introduced into the second antibody chain.

Alternatively or in addition, heterodimerization may be promoted by the introduction of one or more modification(s) to the hydrophilic and hydrophobic residues at the interface between chains, in order make heterodimer formation more entropically and enthalpically favourable than homodimer formation.

A further strategy for promoting heterodimerization is to rearrange portions of the antibody chains such that each chain remains compatible only with a chain comprising corresponding rearrangements. For example, CrossMAb technology is based on the crossover of antibody domains in order to enable correct chain association. There are three main CrossMAb formats, these are: (i) CrossMAb^{Fab} in which the VH and VL are exchanged and the CH1 and CL are exchanged; (ii) CrossMAb ^{VH-VL} in which the VH and VL are exchanged; and (iii) CrossMAb^{CH1-}^{CL} in which the CH1 and CL are exchanged (Klein C et al. (2016) MAbs. 8(6): 1010-1020).

In some embodiments, the multispecific (e.g. bispecific) antibodies of the invention may comprise an exchange of the VH and VL. In some embodiments, the antibodies (e.g. bispecific) antibodies, of the invention may comprise an exchange of the CH1 and CL. In some embodiments, the antibodies (e.g. bispecific) antibodies, of the invention may comprise an exchange of the VH and VL and an exchange of the CH1 and CL.

In preferred embodiments, the multispecific (e.g. bispecific) antibodies of the invention comprise an exchange of the VH and VL.

Other approaches to promoting heterodimerization include the use of a strand exchange engineered domain (SEED) (Davis JH et al. (2010) Protein Eng Des Sel, 23(4): 195- 202).

A combination of the above strategies may be used to maximise the efficiency of assembly while minimising the impact on antibody stability.

### Fc Heterodimerization

In some embodiments, the multispecific (e.g. bispecific) antibodies, of the invention may have a heterodimeric Fc, for example they may comprise one heavy chain originating from an anti-BCMA antibody, and one heavy chain originating from an anti-CD3 antibody.

The multispecific (e.g. bispecific) antibodies, of the invention may comprise a heterodimeric Fc which comprises one or more modification(s) which promotes the association of the first CH2 and/or CH3 domain with the second CH2 and/or CH3 domain. In preferred embodiments, the one or more modification(s) promote the association of the first CH3 domain with the second CH3 domain, for example by resulting in asymmetric modifications to the CH3 domain. The one or more modification(s) may comprise modifications selected from amino acid insertions, deletions, conservative and non-conservative substitutions and rearrangements, and combinations thereof.

Typically the first CH3 domain and the second CH3 domain are both engineered in a complementary manner so that each CH3 domain (or the heavy chain comprising it) can no longer homodimerize with itself but is forced to heterodimerize with the complementary engineered other CH3 domain (so that the first and second CH3 domain heterodimerize and no homodimers between the two first or the two second CH3 domains are formed).

The multispecific (e.g. bispecific) antibodies of the invention may comprise an Fc having one or more of “knob-into-holes” modification(s), which are described in detail with several examples in e.g. WO 96/027011; Ridgway JB et al. (1996) Protein Eng. 9(7) 617-621; Merchant AM et al. (1998) Nat. Biotechnol. 16(7): 677-681; and WO 98/050431.

In this method, the interaction surfaces of the two CH3 domains are altered to increase the heterodimerization of both Fc chains containing these two CH3 domains. One of the two CH3 domains (of the two Fc chains) can be the “knob”, while the other is the “hole”.

Accordingly, the multispecific (e.g. bispecific) antibodies, of the invention may comprise two CH3 domains, wherein the first CH3 domain of the first Fc chain and the second CH3 domain of the second Fc chain each meet at an interface which comprises an original interface between the antibody CH3 domains, wherein said interface is altered to promote the formation of the antibody.

In some embodiments:
a) the CH3 domain of one Fc chain is altered, so that within the original interface of the CH3 domain of the one Fc chain that meets the original interface of the CH3 domain of the other Fc chain, an amino acid residue is replaced with an amino acid residue having a larger side chain volume, thereby generating a protuberance within the interface of the CH3 domain of one Fc chain which is positionable in a cavity within the interface of the CH3 domain of the other Fc chain; and
b) the CH3 domain of the other Fc chain is altered, so that within the original interface of the CH3 domain of the other Fc chain that meets the original interface of the CH3 domain of the one Fc chain, an amino acid residue is replaced with an amino acid residue having a smaller side chain volume, thereby generating a cavity within the interface of the CH3 domain of the other Fc chain within which a protuberance within the interface of the CH3 domain of the one Fc chain is positionable.

Preferably, said amino acid residue having a larger side chain volume is selected from the group consisting of arginine (R), phenylalanine (F), tyrosine (Y), tryptophan (W).

In some embodiments, the multispecific (e.g. bispecific) antibodies of the invention comprise a first CH3 domain comprising modification(s) at positions T366, L368 and Y407, e.g. T366S, L368A, and Y407V (numbered according to EU numbering).

In some embodiments, the multispecific (e.g. bispecific) antibodies of the invention comprise a second CH3 domain comprising a modification at position T366 (“knob modification”), e.g. T366W (numbered according to EU numbering).

In particularly preferred embodiments, the multispecific (e.g. bispecific) antibodies of the invention comprise a first CH3 domain comprising the modifications T366S, L368A, and Y407V, or conservative substitutions thereof, and a second CH3 domain comprising the modification T366W, or a conservative substitution thereof (numbered according to EU numbering).

In one embodiment, the multispecific (e.g. bispecific) antibodies of the invention comprise a first CH3 domain comprising the modification set forth in Table 19 and a second CH3 domain comprising the modifications set forth in Table 19.

TABLE 19

“Knob-into-holes” modification

| First CH3 domain | | Second CH3 domain | |
|---|---|---|---|
| KABAT | EU NUMBERING | KABAT | EU NUMBERING |
| T389S | T366S | T389W | T366W |
| L391A | L368A | | |
| Y438V | Y407V | | |

Alternatively, the multispecific (e.g. bispecific) antibodies of the invention may comprise one or more of the modification(s) set forth in US 9,562,109 and US 9,574,010 (incorporated herein by reference).

In some embodiments, the multispecific (e.g. bispecific) antibodies of the invention comprise a first CH3 domain comprising one or more modification(s) at positions T350, L351, F405 and/or Y407 (numbered according to EU numbering), e.g. T350V, L351Y, F405A and/or Y407V. In some embodiments, the multispecific (e.g. bispecific) antibodies of the invention comprise a first CH3 domain comprising modification(s) at positions T350, L351, F405 and Y407 (numbered according to EU numbering), e.g. T350V, L351Y, F405A and Y407V.

In some embodiments, the multispecific (e.g. bispecific) antibodies of the invention comprise a second CH3 domain comprising one or more modification(s) at positions T350, T366, K392 and/or T394 (numbered according to EU numbering), e.g. T350V, T366L, K392L and/or T394W. In some embodiments, the multispecific (e.g. bispecific) antibodies of the invention comprise a second CH3 domain comprising modification(s) at positions T350, T366, K392 and T394 (numbered according to EU numbering), e.g. T350V, T366L, K392L and T394W.

In preferred embodiments, the multispecific (e.g. bispecific) antibodies of the invention comprise a first CH3 domain comprising one or more modification(s) at positions T350, L351, F405 and/or Y407 (e.g. T350V, L351Y, F405A and/or Y407V) and a second CH3 domain comprising one or more modification(s) at positions T350, T366, K392 and/or T394 (e.g. T350V, T366L, K392L and/or T394W) (numbered according to EU numbering).

In particularly preferred embodiments, the multispecific (e.g. bispecific) antibodies of the invention comprise a first CH3 domain comprising modifications at positions T350, L351, F405 and Y407 (e.g. T350V, L351Y, F405A and Y407V) and a second CH3 domain comprising modifications at positions T350, T366, K392 and T394 (e.g. T350V, T366L, K392L and T394W) (numbered according to EU numbering).

The one or more modification(s) may modify electrostatic charges, hydrophobic/hydrophilic interactions, and/or steric interference between side chains.

In particularly preferred embodiments, the multispecific (e.g. bispecific) antibodies of the invention comprise a first CH3 domain comprising the modifications T350V, L351Y, F405A and Y407V, or conservative substitutions thereof, and a second CH3 domain comprising the modifications T350V, T366L, K392L and T394W, or conservative substitutions thereof (numbered according to EU numbering).

In one embodiment, the multispecific (e.g. bispecific) antibodies of the invention comprise a first CH3 domain comprising the modifications set forth in Table 20 and a second CH3 domain comprising the modifications set forth in Table 20.

TABLE 20

Fc Heterodimerization modifications

| First CH3 domain | | Second CH3 domain | |
|---|---|---|---|
| KABAT | EU NUMBERING | KABAT | EU NUMBERING |
| T371V | T350V | T371V | T350V |
| L372Y | L351Y | T389L | T366L |
| F436A | F405A | K420L | K392L |
| Y438V | Y407V | T422W | T394W |

Other techniques for CH3 modifications to enforce heterodimerization are contemplated as alternatives of the invention and are described e.g. in WO96/27011, WO98/050431, EP1870459, WO2007/110205, WO2007/147901, WO2009/089004, WO2010/129304, WO2011/90754, WO2011/143545, WO2012/058768, WO2013/157954, WO2013/157953, and WO2013/096291.

In some embodiments, the bispecific antibody according to the invention is of IgG2 isotype and the heterodimerization approach described in WO2010/129304 can be used.

### Other Fc Modifications

In some embodiments, the multispecific (e.g. bispecific) antibodies of the invention may comprise an Fc, wherein both CH3 domains are altered by the introduction of cysteine (C) as the amino acid in the corresponding positions of each CH3 domain such that a disulphide bridge between both CH3 domains can be formed. The cysteines may be introduced at position 349 in one of the CH3 domains and at position 354 in the other CH3 domain (numbered according to EU numbering).

Preferably, the cysteine introduced at position 354 is in the first CH3 domain and the cysteine introduced at position 349 is in the second CH3 domain (numbered according to EU numbering).

The Fc may comprise modifications, such as D356E, L358M, N384S, K392N, V397M, and V422I (numbered according to EU numbering). Preferably, both CH3 domains comprise D356E and L358M (numbered according to EU numbering).

### Light and Heavy Chain Heterodimerization

In the multispecific (e.g. bispecific) antibodies of the invention, one or more of the immunoglobulin heavy chains and light chains may comprise one or more modification(s), e.g. amino acid modifications that are capable of promoting preferential pairing of a specific heavy chain with a specific light chain when heavy chains and light chains are co-expressed or coproduced. Such modifications can provide considerably improved production/purification without changing biological properties such as binding to BCMA. In particular, by introduction of one or more modification(s) such as amino acid exchanges, light chain mispairing and the formation of side products in production can be significantly reduced and therefore yield is increased and purification is facilitated.

The one or more modification(s) may promote preferential heterodimer pairing by introducing steric hindrance, substitutions of charged amino acids with opposite charges and/or by hydrophobic or hydrophilic interactions. In preferred embodiments, the one or more modification(s) promote preferential heterodimer pairing by introducing steric hindrance and substitution(s) of charged amino acids with opposite charges.

The amino acid exchanges may be substitutions of charged amino acids with opposite charges (for example in the CH1/CL interface) which reduce light chain mispairing, e.g. Bence-Jones type side products.

In preferred embodiments, the one or more modification(s) assist light and heavy chain heterodimerization are amino acid modifications in the light and heavy chains outside of the CDRs.

The one or more modification(s) may be present in the anti-BCMA antibody or antigen-binding fragment thereof. Alternatively, the one or more modification(s) may be present in the anti-CD3 antibody or antigen-binding fragment thereof. In preferred embodiments, the one or more modification(s) are present in the anti-BCMA antibody or antigen-binding fragment thereof.

In some embodiments, the multispecific (e.g. bispecific) antibodies of the invention comprise an immunoglobulin heavy chain comprising a CH1 domain having amino acid modifications K147E/D and K213E/D (numbered according to EU numbering) and a corresponding immunoglobulin light chain comprising a CL domain having amino acid modifications E123K/R/H and Q124K/R/H (numbered according to Kabat). Preferably, the CH1 domain comprises the amino acid modifications K147E and K213E (numbered according to EU numbering) or conservative substitutions thereof, and the corresponding CL domain comprises the amino acid modifications E123R and Q124K or conservative substitutions thereof (numbered according to Kabat). Such multispecific (e.g. bispecific) antibodies can be produced in high yield and can be easily purified.

In one embodiment, the amino acid modifications described in Table 21 can be in the BCMA antibody or in the CD3 antibody.

In one embodiment, the bispecific antibodies of the invention are bivalent, and comprise one anti-BCMA antibody or antigen-binding fragment thereof and one anti-CD3 antibody or antigen-binding fragment thereof (the “1+1” format), wherein:
a) the BCMA antibody or antigen-binding fragment thereof (e.g. BCMA Fab) comprises a CH1 domain having amino acid modifications set forth in Table 21 and a corresponding CL domain having the amino acid modifications Table 21; or
b) the CD3 antibody or antigen-binding fragment thereof (e.g. CD3 Fab) comprises a CH1 domain having amino acid modifications set forth in Table 21 and a corresponding CL domain having the amino acid modifications Table 21.

In one embodiment, the bispecific antibodies of the invention are trivalent and comprise two anti-BCMA antibodies or antigen-binding fragments thereof and one anti-CD3 antibody or antigen-binding fragment thereof (the “2+1” format), wherein:
a) one or both BCMA antibodies or antigen-binding fragments thereof (e.g. BCMA Fabs) comprises a CH1 domain having amino acid modifications set forth in Table 21 and a corresponding CL domain having the amino acid modifications Table 21; or
b) the CD3 antibody (e.g. CD3 Fab) comprises a CH1 domain having amino acid modifications set forth in Table 21 and a corresponding CL domain having the amino acid modifications Table 21.

In particular, each BCMA antibody (e.g. BCMA Fab) may comprise a CH1 domain having amino acid modifications set forth in Table 21 and a corresponding CL domain having the amino acid modifications Table 21.

TABLE 21

Light and heavy chain heterodimerization modifications

| CH1 domain | | CL domain | |
|---|---|---|---|
| KABAT | EU NUMBERING | KABAT | EU NUMBERING |
| K145E | K147E | E123R | E123R |
| K221E | K213E | Q124K | Q124K |

In a preferred embodiment, the multispecific (e.g. bispecific) antibodies of the invention comprise the modifications set forth in Table 21 in combination with the modifications set forth in Table 19. Thus, in one embodiment, the bispecific antibodies of the invention are bivalent, and comprise:
a) one anti-BCMA antibody or antigen-binding fragment thereof and one anti-CD3 antibody or antigen-binding fragment thereof (the “1+1” format), wherein (i) the BCMA antibody or antigen-binding fragment thereof (e.g. BCMA Fab) comprises a CH1 domain that comprises the amino acid modifications K147E and K213E, and a corresponding CL domain that comprises the amino acid modifications E123R and Q124K (i.e. the modifications set forth in Table 21), or (ii) the CD3 antibody or antigen-binding fragment thereof (e.g. CD3 Fab) comprises a CH1 domain that comprises the amino acid modifications K147E and K213E, and a corresponding CL domain that comprises the amino acid modifications E123R and Q124K (i.e. the modifications set forth in Table 21); and
b) a first CH3 domain comprising the modifications T366S, L368A, and Y407V, and a second CH3 domain comprising the modification T366W (i.e. the modifications set forth in Table 19).

In one embodiment, the bispecific antibodies of the invention are trivalent and comprise:
a) two anti-BCMA antibodies or antigen-binding fragments thereof and one anti-CD3 antibody or antigen-binding fragment thereof (the “2+1” format), wherein (i) one or both BCMA antibodies or antigen-binding fragments thereof (e.g. BCMA Fabs) comprises a CH1 domain that comprises the amino acid modifications K147E and K213E, and a corresponding CL domain that comprises the amino acid modifications E123R and Q124K (i.e. the modifications set forth in Table 21), or (ii) the CD3 antibody or antigen-binding fragment thereof (e.g. CD3 Fab) comprises a CH1 domain that comprises the amino acid modifications K147E and K213E, and a corresponding CL domain that comprises the amino acid modifications E123R and Q124K (i.e. the modifications set forth in Table 21); and
b) a first CH3 domain comprising the modifications T366S, L368A, and Y407V, and a second CH3 domain comprising the modification T366W (i.e. the modifications set forth in Table 19).

In particular, each BCMA antibody (e.g. BCMA Fab) may comprise a CH1 domain having amino acid modifications set forth in Table 21 and a corresponding CL domain having the amino acid modifications Table 21. In preferred embodiments, the first Fc chain is bound at the N-terminus of the Fc to the C-terminus of the first anti-BCMA antibody, and the second Fc chain is bound at the N-terminus of the Fc to the C-terminus of the anti-CD3 antibody.

In alternative embodiments, the multispecific (e.g. bispecific), antibodies of the invention comprise an immunoglobulin heavy chain comprising a CH1 domain having amino acid modifications at one or more of position(s) A141, L145, K147, Q175 (numbered according to EU numbering) and a corresponding immunoglobulin light chain comprising a CL domain having amino acid modifications at one or more of position(s) F116, Q124, L135, T178 (numbered according to Kabat). Preferably, the CH1 domain comprises the amino acid modifications A141W, L145E, K147T, Q175E or conservative substitutions thereof (numbered according to EU numbering), and the corresponding CL domain comprises the amino acid modifications F116A, Q124R, L135V, T178R or conservative substitutions thereof (numbered according to Kabat).

In one embodiment, the multispecific (e.g. bispecific) antibodies of the invention comprise a CH1 domain having amino acid modifications set forth in Table 22 and a corresponding immunoglobulin light chain comprising a CL domain having amino acid modifications set forth in Table 22. In embodiments where the multispecific (e.g. bispecific) antibodies of the invention comprise an anti-BCMA antibody, or antigen binding fragment thereof of the invention, and an anti-CD3 antibody, or antigen binding fragment thereof, of the invention, the amino acid modifications described in Table 22 can be in the BCMA antibody or in the CD3 antibody.

In one embodiment, the bispecific antibodies of the invention are bivalent, and comprise one anti-BCMA antibody and one anti-CD3 antibody (the “1+1” format), wherein:
(a) the BCMA antibody (e.g. BCMA Fab) comprises a CH1 domain having amino acid modifications set forth in Table 22 and a corresponding CL domain having the amino acid modifications Table 22; or
(b) the CD3 antibody (e.g. CD3 Fab) comprises a CH1 domain having amino acid modifications set forth in Table 22 and a corresponding CL domain having the amino acid modifications Table 22.

In one embodiment, the bispecific antibodies of the invention are trivalent and comprise two anti-BCMA antibodies and one anti-CD3 antibody (the “2+1” format), wherein:
(a) one or both BCMA antibodies (e.g. BCMA Fabs) comprises a CH1 domain having amino acid modifications set forth in Table 22 and a corresponding CL domain having the amino acid modifications Table 22; or
(b) the CD3 antibody (e.g. CD3 Fab) comprises a CH1 domain having amino acid modifications set forth in Table 22 and a corresponding CL domain having the amino acid modifications Table 22.

In particularly preferred embodiments, each BCMA antibody (e.g. BCMA Fab) may comprise a CH1 domain having amino acid modifications set forth in Table 22 and a corresponding CL domain having the amino acid modifications Table 22.

TABLE 22

Light and heavy chain heterodimerization modifications

| CH1 domain | | CL domain | |
|---|---|---|---|
| KABAT | EU NUMBERING | KABAT | EU NUMBERING |
| A139W | A141W | F116A | F116A |
| L143E | L145E | Q124R | Q124R |
| K145T | K147T | L135V | L135V |
| Q179E | Q175E | T178R | T178R |

In a preferred embodiment, the multispecific (e.g. bispecific) antibodies of the invention comprise the amino acid modifications set forth in Table 22 in combination with the amino acid modifications set forth in Table 20. Thus, in one embodiment, the bispecific antibodies of the invention are bivalent, and comprise:
(a) one anti-BCMA antibody and one anti-CD3 antibody (the “1+1” format), wherein (i) the BCMA antibody (e.g. BCMA Fab) comprises a CH1 domain that comprises the amino acid modifications A141W, L145E, K147T and Q175E, and a corresponding CL domain that comprises the amino acid modifications F116A, Q124R, L135V and T178R (i.e. the modifications set forth in Table 22), or (ii) the CD3 antibody (e.g. CD3 Fab) comprises a CH1 domain that comprises the amino acid modifications A141W, L145E, K147T and Q175E, and a corresponding CL domain that comprises the amino acid modifications F116A, Q124R, L135V and T178R (i.e. the modifications set forth in Table 22); and
(b) a first CH3 domain comprising the modifications T350V, L351Y, F405A and Y407V, and a second CH3 domain comprising the modifications T350V, T366L, K392L and T394W (i.e. the modifications set forth in Table 20).

In preferred embodiments, the first Fc chain is bound at the N-terminus of the Fc to the C-terminus of the anti-BCMA antibody, and the second Fc chain is bound at the N-terminus of the Fc to the C-terminus of the anti-CD3 antibody.

In one embodiment, the bispecific antibodies of the invention are trivalent and comprise:
(a) two anti-BCMA antibodies and one anti-CD3 antibody (the “2+1” format), wherein (i) one or both BCMA antibodies (e.g. BCMA Fabs) comprises a CH1 domain that comprises the amino acid modifications A141W, L145E, K147T and Q175E, and a corresponding CL domain that comprises the amino acid modifications F116A, Q124R, L135V and T178R (i.e. the modifications set forth in Table 22), or (ii) the CD3 antibody (e.g. CD3 Fab) comprises a CH1 domain that comprises the amino acid modifications A141W, L145E, K147T and Q175E, and a corresponding CL domain that comprises the amino acid modifications F116A, Q124R, L135V and T178R (i.e. the modifications set forth in Table 22); and
(b) a first CH3 domain comprising the modifications T350V, L351Y, F405A and Y407V, and a second CH3 domain comprising the modifications T350V, T366L, K392L and T394W (i.e. the modifications set forth in Table 20).

In particular, each BCMA antibody (e.g. BCMA Fab) comprises a CH1 domain having amino acid modifications set forth in Table 22 and a corresponding CL domain having the amino acid modifications Table 22. In preferred embodiments, the first Fc chain is bound at the N-terminus of the Fc to the C-terminus of the first anti-BCMA antibody, and the second Fc chain is bound at the N-terminus of the Fc to the C-terminus of the anti-CD3 antibody.

Alternatively, the CH1 domain may comprise an amino acid modification at position Q175 (numbered according to EU numbering) and the corresponding CL domain may comprise amino acid modifications at one or more of position(s) F116, Q124, L135, T178 (numbered according to Kabat). The CH1 domain may comprise the amino acid modification Q175K (numbered according to EU numbering), or a conservative substitution thereof, and the corresponding CL domain may comprise amino acid modifications F116A, Q124R, L135V, T178R (numbered according to Kabat), or conservative substitutions thereof.

In alternative embodiments, the CH1 domain may comprises an amino acid modification at position Q175 (numbered according to EU numbering) and the corresponding CL domain may comprise amino acid modifications at one or more of position(s) Q124, L135, Q160, T180 (numbered according to Kabat). The CH1 domain may comprise the amino acid modification Q175K (numbered according to EU numbering), or a conservative substitution thereof, and the corresponding CL domain may comprise the amino acid modifications Q124E, L135W, Q160E and T180E, or conservative substitutions thereof (numbered according to Kabat).

The multispecific (e.g. bispecific) antibodies of the invention may additionally comprise an amino acid substitution at position 49 of the VL region selected from the group of amino acids tyrosine (Y), glutamic acid (E), serine (S), and histidine (H) and/or an amino acid substitution at position 74 of the VL region that is threonine (T) or alanine (A).

### CrossMAb

The multispecific (e.g. bispecific) antibodies of the invention may comprise CrossMAb technology. CrossMAb technology is based on the crossover of antibody domains in order to enable correct chain association. It is used to facilitate multispecific antibody formation. There are three main CrossMAb formats, these are: (i) CrossMAb^{Fab} in which the VH and VL are exchanged and the CH1 and CL are exchanged; (ii) CrossMAb ^{VH-VL} in which the VH and VL are exchanged; and (iii) CrossMAb^{CH1-CL} in which the CH1 and CL are exchanged (Klein C et al. (2016) MAbs. 8(6): 1010-1020).

CrossMAb technology is known in the state of the art. Bispecific antibodies wherein the variable domains VL and VH or the constant domains CL and CH1 are replaced by each other are described in WO2009080251 and WO2009080252.

In one or more of the antibodies or antigen-binding fragments within the multispecific (e.g. bispecific) antibodies of the invention, the variable domains VL and VH or the constant domains CL and CH1 may be replaced by each other. In some embodiments, the antibodies (e.g. bispecific) antibodies, of the invention may comprise an exchange of the VH and VL and an exchange of the CH1 and CL. Thus, the multispecific (e.g. bispecific) antibodies of the invention may comprise a crossover light chain and a crossover heavy chain. As used herein, a “crossover light chain” is a light chain that may comprise a VH-CL, a VL-CH1 or a VH-CH1. A “crossover heavy chain” as used herein is a heavy chain that may comprise a VL-CH1, a VH-CL or a VL-CL.

In some aspects, there is provided a multispecific (e.g. bispecific) antibody comprising an anti-BCMA antibody of the invention, or an antigen-binding fragment thereof, and an anti-CD3 antibody, or antigen-binding fragment thereof, wherein the multispecific (e.g. bispecific) antibody comprises:
a) a light chain and a heavy chain of an antibody specifically binding to CD3; and
b) a light chain and heavy chain of an antibody specifically binding to BCMA,
wherein the variable domains VL and VH and/or the constant domains CL and CH1 are replaced by each other in (i) the anti-BCMA antibody; and/or (ii) the anti-CD3 antibody.

In some embodiments, the variable domains VL and VH or the constant domains CL and CH1 of the anti-CD3 antibody or antigen binding fragment thereof are replaced by each other. More preferably, the variable domains VL and VH of the anti-CD3 antibody or antigen binding fragment thereof are replaced by each other.

In embodiments in which the bispecific antibodies in the 1+1 format have the format: CD3 Fab –BCMA Fab (i.e. when no Fc is present); Fc – CD3 Fab - BCMA Fab; Fc- BCMA Fab – CD3 Fab; or BCMA Fab – Fc – CD3 Fab, the bispecific antibodies may comprise the CrossMAb format, e.g. CrossMAb^{Fab}, CrossMAb ^{VH-VL} or CrossMAb^{CH1}′^{CL}. The BCMA Fab may have the CrossMAb format, e.g. CrossMAb^{Fab}, CrossMAb″ or CrossMAb^{CH1}′^{CL}. Alternatively, the CD3 Fab may have the CrossMAb format, e.g. CrossMAb^{Fab}, CrossMAb ^{VH-VL} or CrossMAb^{CH1-CL}. In preferred embodiments, the CD3 Fab of the bispecific antibody comprises the CrossMAb ^{VH-VL} format.

It is especially preferred for the bispecific antibodies of the invention having the 2+1 format to comprise CrossMAb technology. Thus, in embodiments in which the trivalent bispecific antibodies in the 2+1 format have the format: CD3 Fab – BCMA Fab – BCMA Fab; BCMA Fab –CD3 Fab – BCMA Fab (i.e. when no Fc is present); BCMA Fab – Fc – CD3 Fab – BCMA Fab; BCMA Fab – Fc – BCMA Fab – CD3 Fab; or CD3 Fab – Fc – BCMA Fab - BCMA Fab, the bispecific antibodies may comprise the CrossMAb format, e.g. CrossMAb^{Fab}, CrossMAb ^{VH-VL} or CrossMAb^{CH1}′^{CL}. The BCMA Fab may have the CrossMAb format, e.g. CrossMAb^{Fab}, CrossMAb^{VH-VL} or CrossMAb^{CHI-CL}. Alternatively, the CD3 Fab may have the CrossMAb format, e.g. CrossMAb^{Fab}, CrossMAb ^{VH-VL} or CrossMAb^{CH1}′^{CL}. In preferred embodiments, the CD3 Fab of the bispecific antibody comprises the CrossMAb ^{VH-VL} format.

In some embodiments, the bispecific antibodies of the invention having the 1+1 format do not comprise CrossMAb technology, i.e. neither the anti-BCMA antibody nor the anti-CD3 antibody have the variable domains VL and VH or the constant domains CL and CH1 replaced by each other.

### Exemplary Multispecific Antibodies

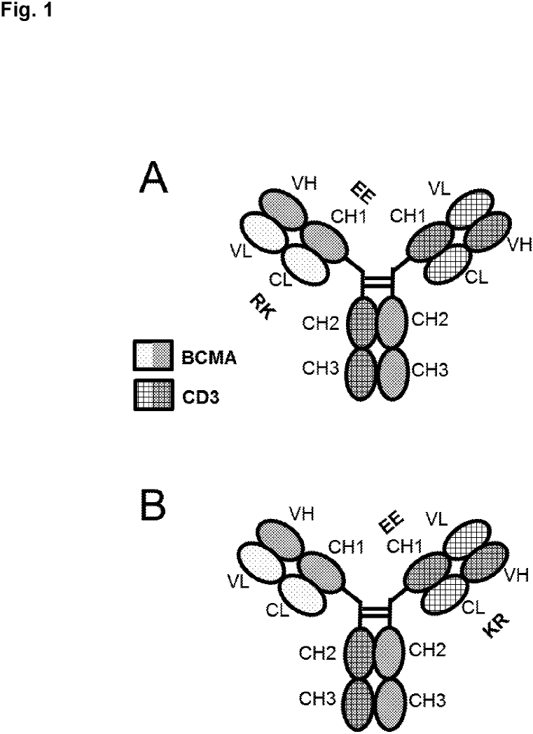

Exemplary embodiments of multispecific antibodies are set out in FIGS. 1-3 and are described below.

In one embodiment, the bispecific antibodies according to the invention are bivalent bispecific antibodies comprising one Fab fragment of an anti-CD3 antibody, one Fab fragment of an anti-BCMA antibody and one Fc part according to the format BCMA Fab – Fc – CD3 Fab. The anti-BCMA Fab fragment comprises the amino acid modifications set forth in Table 21. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 1A.

In one embodiment, the bispecific antibodies according to the invention are bivalent bispecific antibodies comprising one Fab fragment of an anti-CD3 antibody, one Fab fragment of an anti-BCMA antibody and one Fc part according to the format BCMA Fab – Fc – CD3 Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 21. This embodiment is illustrated in FIG. 1B.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab – Fc – CD3 Fab – BCMA Fab. Each anti-BCMA Fab fragment comprises the amino acid modifications set forth in Table 21. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 2A.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab – Fc – CD3 Fab – BCMA Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 21. This embodiment is illustrated in FIG. 2B.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab – Fc – BCMA Fab – CD3 Fab. Each anti-BCMA Fab fragment comprises the amino acid modifications set forth in Table 21. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 2C.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab– Fc – BCMA Fab – CD3 Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 21. This embodiment is illustrated in FIG. 2D.

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc – CD3 Fab – BCMA Fab. The anti-BCMA Fab fragment comprises the amino acid modifications set forth in Table 21. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1.This embodiment is illustrated in FIG. 3A.

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc – CD3 Fab – BCMA Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 21. This embodiment is illustrated in FIG. 3B.

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc – BCMA Fab – CD3 Fab. The anti-BCMA Fab fragment comprises the amino acid modifications set forth in Table 21. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 3C.

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc – BCMA Fab – CD3 Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 21. This embodiment is illustrated in FIG. 3D.

In one embodiment, the antibodies illustrated in FIG. 2 additionally comprise the modifications set forth in Table 19.

In one aspect, the bispecific antibodies according to the invention are trivalent bispecific antibodies comprising one Fab fragment of an anti-CD3 antibody, two Fab fragments of an anti-BCMA antibody and one Fc part according to the format BCMA Fab – Fc –CD3 Fab – BCMA Fab. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. Each anti-BCMA Fab fragment comprises a light chain and heavy chain, wherein the heavy chain comprises a CH1 domain which comprises the amino acid modifications K147E and K213E (numbered according to EU numbering) and wherein the light chain comprises a corresponding CL domain which comprises the amino acid modifications E123R and Q124K (numbered according to Kabat) (i.e. the modifications set forth in Table 21). The Fc part comprises a first Fc chain and a second Fc chain, wherein the first Fc chain comprises a first constant domain CH2 and a first constant domain CH3, and the second Fc chain comprises a second constant domain CH2 and a second constant domain CH3. The first Fc chain is bound at the N-terminus of the Fc to the C-terminus of the first anti-BCMA Fab, and the second Fc chain is bound at the N-terminus of the Fc to the C-terminus of the anti-CD3 Fab. The first CH3 domain comprises the modifications T366S, L368A, and Y407V (“hole modifications”) and the second CH3 domain comprises the modification T366W (“knob modification”) (numbered according to EU numbering) (i.e. the modifications set forth in Table 19). Additionally, both Fc chains further comprise the modifications L234A, L235A and P329G, and optionally D356E and L358M (numbered according to EU numbering). Optionally, the first CH3 domain further comprises the amino acid modification S354C, and the second CH3 domain further comprises the amino acid modification Y349C (numbered according to EU numbering) such that a disulphide bridge between both CH3 domains is formed.

### Pharmaceutical Compositions

The cytokine inhibitor (e.g. IL-6 inhibitor) for use according to the invention can be administered to the patient as a pharmaceutical composition. Accordingly, the present invention also provides a pharmaceutical composition comprising the therapeutically effective dose of the cytokine inhibitor (e.g. IL-6 inhibitor) of the invention and a pharmaceutically acceptable excipient.

The term “pharmaceutically acceptable” as used herein means approved by a regulatory agency of the Federal or a state government, or listed in the U.S. Pharmacopeia, European Pharmacopeia or other generally recognized pharmacopeia for use in animals, and more particularly in humans.

Examples of suitable excipients include one or more of water, saline, phosphate buffered saline, dextrose, glycerol, ethanol, and the like, as well as any combination thereof. In many cases, it will be preferable to include isotonic agents, such as sugars, polyalcohols, or sodium chloride in the composition. In particular, relevant examples of suitable excipients include: (1) Dulbecco’s phosphate buffered saline, pH.about.7.4, containing or not containing about 1 mg/mL to 25 mg/mL human serum albumin, (2) 0.9% saline (0.9% w/v sodium chloride (NaCl)), and (3) 5% (w/v) dextrose; and may also contain an antioxidant such as tryptamine and a stabilizing agent such as Tween 20^{®}.

A person skilled in the art would understand that the appropriate choice of excipient or excipients for use with the therapeutically effective dose of the cytokine inhibitor (e.g. IL-6 inhibitor) of the invention would depend on the desired properties of the pharmaceutical composition.

The pharmaceutical compositions or the therapeutically effective dose of the cytokine inhibitor (e.g. IL-6 inhibitor) of the invention can be administered to a patient by any appropriate systemic or local route of administration. For example, administration may be oral, buccal, sublingual, ophthalmic, intranasal, intratracheal, pulmonary, topical, transdermal, urogenital, rectal, subcutaneous, intravenous, intra-arterial, intraperitoneal, intramuscular, intracranial, intrathecal, epidural, intraventricular or intratumoral. In preferred embodiments, the pharmaceutical compositions or the cytokine inhibitor (e.g. IL-6 inhibitor) is administered orally.

The cytokine inhibitor (e.g. IL-6 inhibitor) may be administered at a fixed dose. In embodiments in which the cytokine inhibitor is iberdomide, the cytokine inhibitor is administered at a fixed dose of between about 0.03 mg to about 6 mg, between about 0.1 mg to about 4 mg, between about 0.3 mg to about 2 mg, or between about 1.0 mg to about 1.6 mg, e.g. about 1.0 mg, about 1.3 mg or about 1.6 mg. In some embodiments, Compound 1 is administered at a fixed dose of between about 0.05 mg to about 5 mg, between about 0.1 mg to about 2 mg, between about 0.2 mg to about 1.6 mg, or between about 0.3 mg to about 1.0 mg, e.g. about 0.3 mg, about 0.6 mg or about 1.0 mg.

Pharmaceutical compositions of the invention can be formulated for administration by any appropriate means, for example by epidermal or transdermal patches, ointments, lotions, creams, or gels; by nebulizers, vaporisers, or inhalers; by injection or infusion; or in the form of capsules, tablets, liquid solutions or suspensions in water or non-aqueous media, drops, suppositories, enemas, sprays, or powders. The most suitable route for administration in any given case will depend on the physical and mental condition of the patient, the nature and severity of the disease, and the desired properties of the formulation.

In a further aspect, the present invention provides a kit comprising:
a) a BCMA therapeutic agent (e.g. a multispecific antibody which specifically binds to BCMA and to an antigen that promotes activation of one or more T cells, e.g. 42-TCBcv); and

b) a pharmaceutical composition comprising Compound 1, wherein Compound 1 is 4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, an isotopolog or a pharmaceutically acceptable salt thereof.

### Combination Therapies

In some embodiments, the treatment comprises the administration of the therapeutically effective dose of the cytokine inhibitor (e.g. IL-6 inhibitor) of the invention to the patient as a combination therapy, wherein the combination therapy comprises the administration of the therapeutically effective dose of the cytokine inhibitor (e.g. IL-6 inhibitor) of the invention and one or more additional therapeutic agents. The term “combination therapy” is meant to encompass administration of the selected therapeutic agents to a single patient, and is intended to include treatments in which the agents are administered by the same or different route of administration or at the same or different time.

In some embodiments, the one or more additional therapeutic agents are selected from the group consisting of:
a) a steroid, e.g. a corticosteroid;
b) an antagonist of a cytokine receptor or cytokine selected from among GM-CSF, IL-10, IL-10R, IL-6, IL-6 receptor (IL-6R), IFNy, IFNGR, IL-2, IL-2R/CD25, MCP-1, CCR2, CCR4, MIPIβ, CCR5, TNFalpha, TNFR1, IL-1, IL-1R1 and IL-IRalpha/IL-1beta, wherein the antagonist is selected from an antibody or antigen-binding fragment, a small molecule, a protein or peptide and a nucleic acid.
c) a molecule that decreases the regulatory T cell (Treg) population, e.g. cyclophosphamide;
d) an antipyretic, analgesics and/or antibiotics; and/or
e) a seizure prophylaxis, e.g. levetiracetam.

As used herein, “corticosteroid” means any naturally occurring or synthetic steroid hormone that can be derived from cholesterol and is characterized by a hydrogenated cyclopentanoperhydrophenanthrene ring system. Naturally occurring corticosteroids are generally produced by the adrenal cortex. Synthetic corticosteroids may be halogenated. Functional groups required for activity include a double bond at Δ4, a C3 ketone, and a C20 ketone. Corticosteroids may have glucocorticoid and/or mineralocorticoid activity. Examples of exemplary corticosteroids include prednisolone, methylprednisolone, prednisone, triamcinolone, betamethasone, budesonide, and dexamethasone. In some embodiments, the corticosteroid is dexamethasone or methylprednisolone.

The antagonist of a cytokine receptor or cytokine may be selected from tocilizumab, siltuximab, anakinra, clazakizumab, sarilumab, olokizumab, elsilimomab, ALD518/BMS-945429, sirukumab (CNTO 136), CPSI-2634, ARGX-109, lenzilumab, FE301 and FM101. In some embodiments, the antagonist is an anti-IL-6R antibody, e.g. tocilizumab. In some embodiments, the antagonist is an anti-IL-6 antibody e.g., siltuximab. In some embodiments, the antagonist is an IL-1R1 antagonist, e.g. anakinra.

In preferred embodiments, the one or more additional therapeutic agents comprises tocilizumab. In alternative preferred embodiments, the one or more additional therapeutic agents comprises anakinra. In some embodiments, the one or more additional therapeutic agents comprises tocilizumab and anakinra.

In some embodiments, the present invention provides a method for treating or preventing cytokine release syndrome (CRS) in a subject, the method comprising administering to the subject pomalidomide and tocilizumab and/or anakinra.

In some embodiments, the present invention provides a method for treating or preventing cytokine release syndrome (CRS) in a subject, the method comprising administering to the subject iberdomide and tocilizumab and/or anakinra.

In some embodiments, the present invention provides a method for treating or preventing cytokine release syndrome (CRS) in a subject, the method comprising administering to the subject lenalidomide and tocilizumab and/or anakinra.

In some embodiments, the present invention provides a method for treating or preventing cytokine release syndrome (CRS) in a subject, the method comprising administering to the subject avadomide and tocilizumab and/or anakinra.

In some embodiments, the present invention provides a method for treating or preventing cytokine release syndrome (CRS) in a subject, the method comprising administering to the subject a therapeutically effective dose of Compound 1 and tocilizumab and/or anakinra, and wherein Compound 1 is 4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, an isotopolog or a pharmaceutically acceptable salt thereof.

In preferred embodiments, the one or more additional therapeutic agents comprises dexamethasone. The cytokine inhibitor (e.g. IL-6 inhibitor) of the invention may be administered consecutively (before or after) or concurrently with the dexamethasone. In some embodiments, if an adverse event (e.g. CRS or neutropenia) occurs the treatment may further comprise administering to the patient dexamethasone.

The dexamethasone may be administered at an amount sufficient to attenuate secretion of cytokines (e.g. IL-6) induced by a T cell engager described herein. In some embodiments, the dexamethasone is administered at a dose of about 20 mg to about 40 mg weekly. For example, a dose of about 40 mg / week dexamethasone may be administered to non-elderly subjects, e.g. up to and including 75 years old, whereas a dose of about 20 mg / week dexamethasone may be administered to elderly subjects, e.g. over the age of 75 or subjects that are underweight.

Preferably, the dexamethasone is administered orally or intravenously. In some embodiments, the dexamethasone is administered orally with the cytokine inhibitor (e.g. IL-6 inhibitor). In some embodiments, the dexamethasone is administered intravenously on a day on which the T cell engager (e.g. BCMA therapeutic agent) is administered. The dexamethasone may be administered for at least one month following administration of the T cell engager, e.g. for two or preferably three months following administration of the T cell engager.

In an aspect, the present invention provides a method for treating or preventing cytokine release syndrome (CRS) in a subject, the method comprising administering to the subject iberdomide and dexamethasone.

In an aspect, the present invention provides a method for treating or preventing cytokine release syndrome (CRS) in a subject, the method comprising administering to the subject Compound 1 and dexamethasone.

In an aspect, the present invention provides a method for treating or preventing cytokine release syndrome (CRS) in a subject, the method comprising administering to the subject pomalidomide and dexamethasone.

In an aspect, the present invention provides a method for treating or preventing cytokine release syndrome (CRS) in a subject, the method comprising administering to the subject lenalidomide and dexamethasone.

In an aspect, the present invention provides a method for treating or preventing cytokine release syndrome (CRS) in a subject, the method comprising administering to the subject thalidomide and dexamethasone.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

In the context of the present invention other examples and variations of the antibodies and methods described herein will be apparent to a person of skill in the art. Other examples and variations are within the scope of the invention, as set out in the appended claims.

All documents cited herein are each entirely incorporated by reference herein, including all data, tables, figures, and text presented in the cited documents.

TABLE 23A

Antibody sequences

| SEQ ID NO: | Name(s) | Amino acid sequences |
|---|---|---|
| 1 | CD3 CDR1H | TYAMN |
| 2 | CD3 CDR2H | RIRSKYNNYATYYADSVKG |
| 3 | CD3 CDR3H | HGNFGNSYVSWFAY |
| 4 | CD3 CDR1L | GSSTGAVTTSNYAN |
| 5 | CD3 CDR2L | GTNKRAP |
| 6 | CD3 CDR3L | ALWYSNLWV |
| 7 | CD3 VH | EVQLLESGGGLVQPGGSLRLSCAASGFTFSTYAMNWVR QAPGKGLEWVSRIRSKYNNYATYYADSVKGRFTISRDD SKNTLYLQMNSLRAEDTAVYYCVRHGNFGNSYVSWFA YWGQGTLVTVSS |
| 8 | CD3 VL | QAWTQEPSLTVSPGGTVTLTCGSSTGAVTTSNYANWV QEKPGQAFRGLIGGTNKRAPGTPARFSGSLLGGKAALTL SGAQPEDEAEYYCALWYSNLWVFGGGTKLTVL |
| 9 | 83A10 VH | EVQLLESGGGLVQPGGSLRLSCAASGFTFSSYAMSWVR QAPGKGLEWVSAISGSGGSTYYADSVKGRFTISRDNSKN TLYLQMNSLRAEDTAVYYCAKVLGWFDYWGQGTLVT VSS |
| 10 | Mab21 VH Mab22 VH Mab42 VH | EVQLLESGGGLVQPGGSLRLSCAASGFTFSDNAMGWVR QAPGKGLEWVSAISGPGSSTYYADSVKGRFTISRDNSKN TLYLQMNSLRAEDTAVYYCAKVLGWFDYWGQGTLVT |
| | | VSS |
| 11 | 83A10 VL | EIVLTQSPGTLSLSPGERATLSCRASQSVSSSYLAWYQQ KPGQAPRLLIYGASSRATGIPDRFSGSGSGTDFTLTISRLE PEDFAVYYCQQYGYPPDFTFGQGTKVEIK |
| 12 | Mab21 VL Mab27 VL Mab33 VL Mab39 VL | EIVLTQSPGTLSLSPGERATLSCRASQSVSEYYLAWYQQ KPGQAPRLLIEHASTRATGIPDRFSGSGSGTDFTLTISRLE PEDFAVYYCQQYGYPPDFTFGQGTKVEIK |
| 13 | Mab22 VL | EIVLTQSPGTLSLSPGERATLSCRASQSVSSYYLAWYQQ KPGQAPRLLISGAGSRATGIPDRFSGSGSGTDFTLTISRLE PEDFAVYYCQQYGYPPDFTFGQGTKVEIK |
| 14 | Mab42 VL | EIVLTQSPGTLSLSPGERATLSCRASQSVSDEYLSWYQQ KPGQAPRLLIHSASTRATGIPDRFSGSGSGTDFTLAISRLE PEDFAVYYCQQYGYPPDFTFGQGTKVEIK |
| 15 | 83A10 CDR1H | SYAMS |
| 16 | 83A10 CDR2H | AISGSGGSTYYADSVKG |
| 17 | 83A10 CDR3H Mab21 CDR3H Mab22 CDR3H Mab42 CDR3H Mab27 CDR3H Mab33 CDR3H Mab39 CDR3H | VLGWFDY |
| 18 | 83A10 CDR1L | RASQSVSSSYLA |
| 19 | 83A10 CDR2L | GASSRAT |
| 20 | 83A10 CDR3L Mab21 CDR3L Mab22 CDR3L Mab42 CDR3L | QQYGYPPDFT |
| 21 | Mab21 CDR1H Mab22 CDR1H Mab42 CDR1H | DNAMG |
| 22 | Mab21 CDR2H Mab22 CDR2H Mab42 CDR2H | AISGPGSSTYYADSVKG |
| 23 | Mab21 CDR1L | RASQSVSEYYLAW |
| 24 | Mab21 CDR2L | EHASTRAT |
| 25 | Mab22 CDR1L | RASQSVSSYYLA |
| 26 | Mab22 CDR2L | GAGSRAT |
| 27 | Mab42 CDR1L | RASQSVSDEYLS |
| 28 | Mab42 CDR2L | SASTRAT |
| 29 | Mab27 CDR1H | SAPMG |
| 30 | Mab27 CDR2H | AISYIGHTYYADSVKG |
| 31 | Mab27 CDR1L Mab33 CDR1L Mab39 CDR1L | RASQSVSEYYLA |
| 32 | Mab27 CDR2L Mab33 CDR2L Mab39 CDR2L | HASTRAT |
| 33 | Mab27 CDR3L Mab33 CDR3L Mab39 CDR3L | QQYGYPPDFT |
| 34 | Mab33 CDR1H | TNAMG |
| 35 | Mab33 CDR2H | AINRFGGSTYYADSVKG |
| 36 | Mab39 CDR1H | QNAMG |
| 37 | Mab39 CDR2H | AISPTGFSTYYADSVKG |
| 38 | Mab27 VH | EVQLLESGGGLVQPGGSLRLSCAASGFTFSSAPMGWVR QAPGKGLEWVSAISYIGHTYYADSVKGRFTISRDNSKNT LYLQMNSLRAEDTAVYYCAKVLGWFDYWGQGTLVTV SS |
| 39 | Mab33 VH | EVQLLESGGGLVQPGGSLRLSCAASGFTFYTNAMGWVR QAPGKGLEWVSAINRFGGSTYYADSVKGRFTISRDNSK NTLYLQMNSLRAEDTAVYYCAKVLGWFDYWGQGTLV TVSS |
| 40 | Mab39 VH | EVQLLESGGGLVQPGGSLRLSCAASGFTFTQNAMGWVR QAPGKGLEWVSAISPTGFSTYYADSVKGRFTISRDNSKN TLYLQMNSLRAEDTAVYYCAKVLGWFDYWGQGTLVT VSS |
| 41 | 83A10 BCMA CH1 Mab21 BCMA CH1 Mab22 BCMA CH1 Mab42 BCMA CH1 | ASTKGPSVFPLAPSSKSTSGGTAALGCLVEDYFPEPVTVS WNSGALTSGVHTFPAVLQSSGLYSLSSVVTVPSSSLGTQ TYICNVNHKPSNTKVDEKVEPKSC |
| 42 | 83A10 BCMA CL Mab21 BCMA CL Mab22 BCMA CL Mab42 BCMA CL | RTVAAPSVFIFPPSDRKLKSGTASVVCLLNNFYPREAKV QWKVDNALQSGNSQESVTEQDSKDSTYSLSSTLTLSKA DYEKHKVYACEVTHQGLSSPVTKSFNRGEC |
| 43 | CD3 CH1 | ASTKGPSVFPLAPSSKSTSGGTAALGCLVKDYFPEPVTV SWNSGALTSGVHTFPAVLQSSGLYSLSSVVTVPSSSLGT QTYICNVNHKPSNTKVDKKVEPKSC |
| 44 | CD3 CL | ASVAAPSVFIFPPSDEQLKSGTASVVCLLNNFYPREAKV QWKVDNALQSGNSQESVTEQDSKDSTYSLSSTLTLSKA DYEKHKVYACEVTHQGLSSPVTKSFNRGEC |
| 45 | 83A10 knob HC | EVQLLESGGGLVQPGGSLRLSCAASGFTFSSYAMSWVR QAPGKGLEWVSAISGSGGSTYYADSVKGRFTISRDNSKN TLYLQMNSLRAEDTAVYYCAKVLGWFDYWGQGTLVT VSSASTKGPSVFPLAPSSKSTSGGTAALGCLVEDYFPEPV TVSWNSGALTSGVHTFPAVLQSSGLYSLSSVVTVPSSSL GTQTYICNVNHKPSNTKVDEKVEPKSCDGGGGSGGGGS QAWTQEPSLTVSPGGTVTLTCGSSTGAVTTSNYANWV QEKPGQAFRGLIGGTNKRAPGTPARFSGSLLGGKAALTL SGAQPEDEAEYYCALWYSNLWVFGGGTKLTVLSSASTK GPSVFPLAPSSKSTSGGTAALGCLVKDYFPEPVTVSWNS GALTSGVHTFPAVLQSSGLYSLSSVVTVPSSSLGTQTYIC NVNHKPSNTKVDKKVEPKSCDKTHTCPPCPAPEAAGGP SVFLFPPKPKDTLMISRTPEVTCWVDVSHEDPEVKFNW YVDGVEVHNAKTKPREEQYNSTYRVVSVLTVLHQDWL NGKEYKCKVSNKALGAPIEKTISKAKGQPREPQVYTLPP CRDELTKNQVSLWCLVKGFYPSDIAVEWESNGQPENNY KTTPPVLDSDGSFFLYSKLTVDKSRWQQGNVFSCSVMH EALHNHYTQKSLSLSPGK |
| 46 | 83A10 hole HC | EVQLLESGGGLVQPGGSLRLSCAASGFTFSSYAMSWVR QAPGKGLEWVSAISGSGGSTYYADSVKGRFTISRDNSKN TLYLQMNSLRAEDTAVYYCAKVLGWFDYWGQGTLVT VSSASTKGPSVFPLAPSSKSTSGGTAALGCLVEDYFPEPV TVSWNSGALTSGVHTFPAVLQSSGLYSLSSVVTVPSSSL GTQTYICNVNHKPSNTKVDEKVEPKSCDKTHTCPPCPAP EAAGGPSVFLFPPKPKDTLMISRTPEVTCWVDVSHEDP EVKFNWYVDGVEVHNAKTKPREEQYNSTYRVVSVLTV LHQDWLNGKEYKCKVSNKALGAPIEKTISKAKGQPREP QVCTLPPSRDELTKNQVSLSCAVKGFYPSDIAVEWESNG QPENNYKTTPPVLDSDGSFFLVSKLTVDKSRWQQGNVF SCSVMHEALHNHYTQKSLSLSPGK |
| 47 | 83A10 LC | EIVLTQSPGTLSLSPGERATLSCRASQSVSSSYLAWYQQ KPGQAPRLLIYGASSRATGIPDRFSGSGSGTDFTLTISRLE PEDFAVYYCQQYGYPPDFTFGQGTKVEIKRTVAAPSVFI FPPSDRKLKSGTASVVCLLNNFYPREAKVQWKVDNALQ SGNSQESVTEQDSKDSTYSLSSTLTLSKADYEKHKVYAC EVTHQGLSSPVTKSFNRGEC |
| 48 | CD3 LC | EVQLLESGGGLVQPGGSLRLSCAASGFTFSTYAMNWVR QAPGKGLEWVSRIRSKYNNYATYYADSVKGRFTISRDD SKNTLYLQMNSLRAEDTAVYYCVRHGNFGNSYVSWFA YWGQGTLVTVSSASVAAPSVFIFPPSDEQLKSGTASVVC LLNNFYPREAKVQWKVDNALQSGNSQESVTEQDSKDST YSLSSTLTLSKADYEKHKVYACEVTHQGLSSPVTKSFNR GEC |
| 49 | Mab21 knob HC | EVQLLESGGGLVQPGGSLRLSCAASGFTFSDNAMGWVR QAPGKGLEWVSAISGPGSSTYYADSVKGRFTISRDNSKN TLYLQMNSLRAEDTAVYYCAKVLGWFDYWGQGTLVT VSSASTKGPSVFPLAPSSKSTSGGTAALGCLVEDYFPEPV TVSWNSGALTSGVHTFPAVLQSSGLYSLSSVVTVPSSSL GTQTYICNVNHKPSNTKVDEKVEPKSCDGGGGSGGGGS QAWTQEPSLTVSPGGTVTLTCGSSTGAVTTSNYANWV QEKPGQAFRGLIGGTNKRAPGTPARFSGSLLGGKAALTL SGAQPEDEAEYYCALWYSNLWVFGGGTKLTVLSSASTK GPSVFPLAPSSKSTSGGTAALGCLVKDYFPEPVTVSWNS GALTSGVHTFPAVLQSSGLYSLSSVVTVPSSSLGTQTYIC NVNHKPSNTKVDKKVEPKSCDKTHTCPPCPAPEAAGGP SVFLFPPKPKDTLMISRTPEVTCWVDVSHEDPEVKFNW YVDGVEVHNAKTKPREEQYNSTYRVVSVLTVLHQDWL NGKEYKCKVSNKALGAPIEKTISKAKGQPREPQVYTLPP CRDELTKNQVSLWCLVKGFYPSDIAVEWESNGQPENNY KTTPPVLDSDGSFFLYSKLTVDKSRWQQGNVFSCSVMH EALHNHYTQKSLSLSPGK |
| 50 | Mab21 hole HC | EVQLLESGGGLVQPGGSLRLSCAASGFTFSDNAMGWVR QAPGKGLEWVSAISGPGSSTYYADSVKGRFTISRDNSKN TLYLQMNSLRAEDTAVYYCAKVLGWFDYWGQGTLVT VSSASTKGPSVFPLAPSSKSTSGGTAALGCLVEDYFPEPV TVSWNSGALTSGVHTFPAVLQSSGLYSLSSVVTVPSSSL GTQTYICNVNHKPSNTKVDEKVEPKSCDKTHTCPPCPAP EAAGGPSVFLFPPKPKDTLMISRTPEVTCWVDVSHEDP EVKFNWYVDGVEVHNAKTKPREEQYNSTYRVVSVLTV LHQDWLNGKEYKCKVSNKALGAPIEKTISKAKGQPREP QVCTLPPSRDELTKNQVSLSCAVKGFYPSDIAVEWESNG QPENNYKTTPPVLDSDGSFFLVSKLTVDKSRWQQGNVF SCSVMHEALHNHYTQKSLSLSPGK |
| 51 | Mab21 LC | EIVLTQSPGTLSLSPGERATLSCRASQSVSEYYLAWYQQ KPGQAPRLLIEHASTRATGIPDRFSGSGSGTDFTLTISRLE PEDFAVYYCQQYGYPPDFTFGQGTKVEIKRTVAAPSVFI FPPSDRKLKSGTASVVCLLNNFYPREAKVQWKVDNALQ SGNSQESVTEQDSKDSTYSLSSTLTLSKADYEKHKVYAC EVTHQGLSSPVTKSFNRGEC |
| 52 | Mab22 knob HC | EVQLLESGGGLVQPGGSLRLSCAASGFTFSDNAMGWVR QAPGKGLEWVSAISGPGSSTYYADSVKGRFTISRDNSKN TLYLQMNSLRAEDTAVYYCAKVLGWFDYWGQGTLVT VSSASTKGPSVFPLAPSSKSTSGGTAALGCLVEDYFPEPV TVSWNSGALTSGVHTFPAVLQSSGLYSLSSVVTVPSSSL GTQTYICNVNHKPSNTKVDEKVEPKSCDGGGGSGGGGS QAWTQEPSLTVSPGGTVTLTCGSSTGAVTTSNYANWV QEKPGQAFRGLIGGTNKRAPGTPARFSGSLLGGKAALTL SGAQPEDEAEYYCALWYSNLWVFGGGTKLTVLSSASTK GPSVFPLAPSSKSTSGGTAALGCLVKDYFPEPVTVSWNS GALTSGVHTFPAVLQSSGLYSLSSVVTVPSSSLGTQTYIC NVNHKPSNTKVDKKVEPKSCDKTHTCPPCPAPEAAGGP SVFLFPPKPKDTLMISRTPEVTCWVDVSHEDPEVKFNW YVDGVEVHNAKTKPREEQYNSTYRVVSVLTVLHQDWL NGKEYKCKVSNKALGAPIEKTISKAKGQPREPQVYTLPP CRDELTKNQVSLWCLVKGFYPSDIAVEWESNGQPENNY KTTPPVLDSDGSFFLYSKLTVDKSRWQQGNVFSCSVMH EALHNHYTQKSLSLSPGK |
| 53 | Mab22 hole HC | EVQLLESGGGLVQPGGSLRLSCAASGFTFSDNAMGWVR QAPGKGLEWVSAISGPGSSTYYADSVKGRFTISRDNSKN TLYLQMNSLRAEDTAVYYCAKVLGWFDYWGQGTLVT VSSASTKGPSVFPLAPSSKSTSGGTAALGCLVEDYFPEPV TVSWNSGALTSGVHTFPAVLQSSGLYSLSSVVTVPSSSL GTQTYICNVNHKPSNTKVDEKVEPKSCDKTHTCPPCPAP EAAGGPSVFLFPPKPKDTLMISRTPEVTCWVDVSHEDP EVKFNWYVDGVEVHNAKTKPREEQYNSTYRVVSVLTV LHQDWLNGKEYKCKVSNKALGAPIEKTISKAKGQPREP QVCTLPPSRDELTKNQVSLSCAVKGFYPSDIAVEWESNG QPENNYKTTPPVLDSDGSFFLVSKLTVDKSRWQQGNVF SCSVMHEALHNHYTQKSLSLSPGK |
| 54 | Mab22 LC | EIVLTQSPGTLSLSPGERATLSCRASQSVSSYYLAWYQQ KPGQAPRLLISGAGSRATGIPDRFSGSGSGTDFTLTISRLE PEDFAVYYCQQYGYPPDFTFGQGTKVEIKRTVAAPSVFI FPPSDRKLKSGTASVVCLLNNFYPREAKVQWKVDNALQ SGNSQESVTEQDSKDSTYSLSSTLTLSKADYEKHKVYAC EVTHQGLSSPVTKSFNRGEC |
| 55 | Mab42 knob HC | EVQLLESGGGLVQPGGSLRLSCAASGFTFSDNAMGWVR QAPGKGLEWVSAISGPGSSTYYADSVKGRFTISRDNSKN TLYLQMNSLRAEDTAVYYCAKVLGWFDYWGQGTLVT VSSASTKGPSVFPLAPSSKSTSGGTAALGCLVEDYFPEPV TVSWNSGALTSGVHTFPAVLQSSGLYSLSSVVTVPSSSL GTQTYICNVNHKPSNTKVDEKVEPKSCDGGGGSGGGGS QAWTQEPSLTVSPGGTVTLTCGSSTGAVTTSNYANWV QEKPGQAFRGLIGGTNKRAPGTPARFSGSLLGGKAALTL SGAQPEDEAEYYCALWYSNLWVFGGGTKLTVLSSASTK GPSVFPLAPSSKSTSGGTAALGCLVKDYFPEPVTVSWNS GALTSGVHTFPAVLQSSGLYSLSSVVTVPSSSLGTQTYIC NVNHKPSNTKVDKKVEPKSCDKTHTCPPCPAPEAAGGP SVFLFPPKPKDTLMISRTPEVTCWVDVSHEDPEVKFNW YVDGVEVHNAKTKPREEQYNSTYRVVSVLTVLHQDWL NGKEYKCKVSNKALGAPIEKTISKAKGQPREPQVYTLPP CRDELTKNQVSLWCLVKGFYPSDIAVEWESNGQPENNY KTTPPVLDSDGSFFLYSKLTVDKSRWQQGNVFSCSVMH EALHNHYTQKSLSLSPGK |
| 56 | Mab42 hole HC | EVQLLESGGGLVQPGGSLRLSCAASGFTFSDNAMGWVR QAPGKGLEWVSAISGPGSSTYYADSVKGRFTISRDNSKN TLYLQMNSLRAEDTAVYYCAKVLGWFDYWGQGTLVT VSSASTKGPSVFPLAPSSKSTSGGTAALGCLVEDYFPEPV TVSWNSGALTSGVHTFPAVLQSSGLYSLSSVVTVPSSSL GTQTYICNVNHKPSNTKVDEKVEPKSCDKTHTCPPCPAP EAAGGPSVFLFPPKPKDTLMISRTPEVTCWVDVSHEDP EVKFNWYVDGVEVHNAKTKPREEQYNSTYRVVSVLTV LHQDWLNGKEYKCKVSNKALGAPIEKTISKAKGQPREP QVCTLPPSRDELTKNQVSLSCAVKGFYPSDIAVEWESNG QPENNYKTTPPVLDSDGSFFLVSKLTVDKSRWQQGNVF SCSVMHEALHNHYTQKSLSLSPGK |
| 57 | Mab42 LC | EIVLTQSPGTLSLSPGERATLSCRASQSVSDEYLSWYQQ KPGQAPRLLIHSASTRATGIPDRFSGSGSGTDFTLAISRLE PEDFAVYYCQQYGYPPDFTFGQGTKVEIKRTVAAPSVFI FPPSDRKLKSGTASVVCLLNNFYPREAKVQWKVDNALQ SGNSQESVTEQDSKDSTYSLSSTLTLSKADYEKHKVYAC EVTHQGLSSPVTKSFNRGEC |
| 58 | 83A10 CDR1L (alternative to SEQ ID NO: 18) | RASQSVSSSYLAW |
| 59 | 83A10 CDR2L (alternative to SEQ ID NO: 19) | YGASSRAT |
| 60 | Mab22 CDR1L (alternative to SEQ ID NO: 25) | RASQSVSSYYLAW |
| 61 | Mab22 CDR2L (alternative to SEQ ID NO: 26) | SGAGSRAT |
| 62 | Mab42 CDR1L (alternative to SEQ ID NO: 27) | RASQSVSDEYLSW |
| 63 | Mab42 CDR2L (alternative to SEQ ID NO: 28) | HSASTRAT |
| 64 | C11D5.3 CDR1H | DYSIN |
| 65 | C11D5.3 CDR2H | WINTETREPAYAYDFRG |
| 66 | C11D5.3 CDR3H | DYSYAMDY |
| 67 | C11D5.3 CDR1 L1 | RASESVSVIGAHLIH |
| 68 | C11D5.3 CDR2 L1 | LASNLET |
| 69 | C11D5.3 CDR3 L1 | LQSRIFPRT |
| 70 | C11D5.3 CDR1 L2 | RASESVDNYGFSFMH |
| 71 | C11D5.3 CDR2 L2 | RASNLES |
| 72 | C11D5.3 CDR3 L2 | QQSNKDPRT |
| 73 | C11D5.3 CDR1 L3 | RASESVTILGSHLIH |
| 74 | C11D5.3 CDR2 L3 | LASNVQT |
| 75 | C11D5.3 CDR3 L3 | LQSRTIPRT |
| 76 | C11D5.3 VH | QIQLVQSGPELKKPGETVKISCKASGYTFTDYSINWVKR APGKGLKWMGWINTETREPAYAYDFRGRFAFSLETSAS TAYLQINNLKYEDTATYFCALDYSYAMDYWGQGTSVT VSS |
| 77 | C11D5.3 VL1 | DIVLTQSPPSLAMSLGKRATISCRASESVTILGSHLIHWY QQKPGQPPTLLIQLASNVQTGVPARFSGSGSRTDFTLTID PVEEDDVAVYYCLQSRTIPRTFGGGTKLEIK |
| 78 | C11D5.3 VL2 | DIVLTQSPASLAVSLGQRATISCRASESVDNYGFSFMHW YQQKPGQPPKLLIYRASNLESGIPARFSGSGSRTDFALTI NPVETDDVATYYCQQSNKDPRTFGGGTKLEIK |
| 79 | C11D5.3 VL3 | DIVLTQSPASLAMSLGKRATISCRASESVSVIGAB_{L}IRWY QQKPGQPPKLLIYLASNLETGVPARFSGSGSGTDFTLTID PVEEDDVAIYSCLQSRIFPRTFGGGTKLEIK |
| 80 | A7D12.2 VH | QIQLVQSGPDLKKPGETVKLSCKASGYTFTNFGMNWVK QAPGKGFKWMAWINTYTGESYFADDFKGRFAFSVETSA TTAYLQINNLKTEDTATYFCARGEIYYGYDGGFAYWGQ GTLVTVSA |
| 81 | A7D12.2 VL | DVVMTQSHRFMSTSVGDRVSITCRASQDVNTAVSWYQ QKPGQSPKLLIFSASYRYTGVPDRFTGSGSGADFTLTISS VQAEDLAVYYCQQHYSTPWTFGGGTKLDIK |
| 82 | C12A32.2 VH | QIQLVQSGPELKKPGETVKISCKASGYTFRHYSMNWVK QAPGKGLKWMGRINTESGVPIYADDFKGRFAFSVETSA STAYLVINNLKDEDTASYFCSNDYLYSLDFWGQGTALT VSS |
| 83 | C12A32.2 VL | DIVLTQSPPSLAMSLGKRATISCRASESVTILGSHLIYWY QQKPGQPPTLLIQLASNVQTGVPARFSGSGSRTDFTLTID PVEEDDVAVYYCLQSRTIPRTFGGGTKLEIK |
| 84 | C13F12.1 VH | QIQLVQSGPELKKPGETVKISCKASGYTFTHYSMNWVK QAPGKGLKWMGRINTETGEPLYADDFKGRFAFSLETSA STAYLVINNLKNEDTATFFCSNDYLYSCDYWGQGTTLT VSS |
| 85 | C13F12.1 VL | DIVLTQSPPSLAMSLGKRATISCRASESVTILGSHLIYWY QQKPGQPPTLLIQLASNVQTGVPARFSGSGSRTDFTLTID PVEEDDVAVYYCLQSRTIPRTFGGGTKLEIK |

Remarks: SEQ ID NO:20 and SEQ ID NO:33 are identical; SEQ ID NO: 83 and SEQ ID NO: 85 are identical.

TABLE 23B

Anti-BCMA sequences (short list)
SEQ ID NO:

| CD3 antibody | VH | VL | CDR1H | CDR2H | CDR3H | CDR1L | CDR2L | CDR3L |
|---|---|---|---|---|---|---|---|---|
| | 7 | 8 | 1 | 2 | 3 | 4 | 5 | 6 |
| BCMA antibody | VH | VL | CDR1H | CDR2H | CDR3H | CDR1L | CDR2L | CDR3L |
| 83A10 | 9 | 11 | 15 | 16 | 17 | 18 | 19 | 20 |
| Mab21 | 10 | 12 | 21 | 22 | 17 | 23 | 24 | 20 |
| Mab22 | 10 | 13 | 21 | 22 | 17 | 25 | 26 | 20 |
| Mab42 | 10 | 14 | 21 | 22 | 17 | 27 | 28 | 20 |
| Mab27 | 38 | 12 | 29 | 30 | 17 | 31 | 32 | 33 |
| Mab33 | 39 | 12 | 34 | 35 | 17 | 31 | 32 | 33 |
| Mab39 | 40 | 12 | 36 | 37 | 17 | 31 | 32 | 33 |
| C11D5.3 A | 76 | 77 | 64 | 65 | 66 | 67 | 68 | 69 |
| C11D5.3 B | 76 | 78 | 64 | 65 | 66 | 70 | 71 | 72 |
| C11D5.3 C | 76 | 79 | 64 | 65 | 66 | 73 | 74 | 75 |

TABLE 24A

Additional constructs
SEQ ID NO:

| Fragment/Construct | 83A10 | Mab21 | Mab22 | Mab42 |
|---|---|---|---|---|
| BCMA CH1 | 41 | 41 | 41 | 41 |
| BCMA CL | 42 | 42 | 42 | 42 |
| CD3 CH1 | 43 | 43 | 43 | 43 |
| CD3 CL | 44 | 44 | 44 | 44 |

TABLE 24B

Additional constructs
SEQ ID NO:

| Construct | 83A10 | Mab21 | Mab22 | Mab42 |
|---|---|---|---|---|
| BCMA VH _CH1cv x CD3 VL_CH1 Fc knob LALA PG (knob HC) | 45 | 49 | 52 | 55 |
| BCMAcv HC hole LALA PG (hole HC) | 46 | 50 | 53 | 56 |
| BCMAcv hum IgGl LC (BCMA LC) | 47 | 51 | 54 | 57 |
| CD3 VH CL (CD3 LC) | 48 | 48 | 48 | 48 |

### EXAMPLES

### Example 1: Treatment With Lenalidomide, Pomalidomide, Iberdomide or Compound 1 Reduces IL-6 Secretion from Isolated Monocytes

eripheral blood mononuclear cells (PBMCs) were isolated from the blood of four healthy human donors by Ficoll cell separation, and then monocytes were isolated from these PBMCs by negative selection. The enriched monocytes were seeded at a concentration of 1×10⁶ cells/ml and incubated overnight (~17 hours) with 1000 nM lenalidomide, 100 nM pomalidomide, 10 nM iberdomide or 1 nM Compound 1, or control DMSO (0.001%), before stimulation with various concentrations of LPS for IL-6 secretion.

After 4 hours, the cell culture was spun down at 300 g for 5 minutes to remove cells, and the supernatant was subjected to Meso Scale Discovery (MSD) analysis to quantify secreted IL-6.

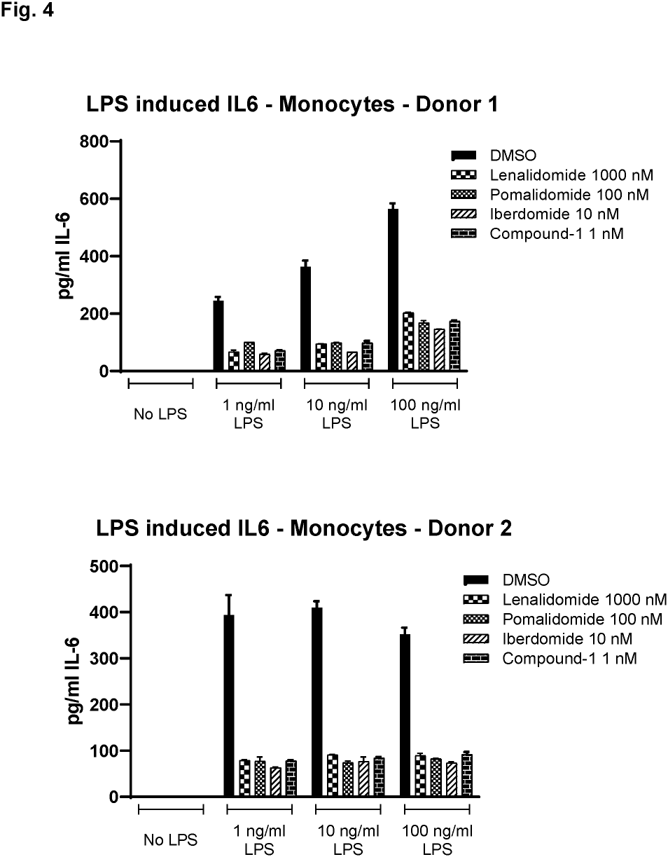

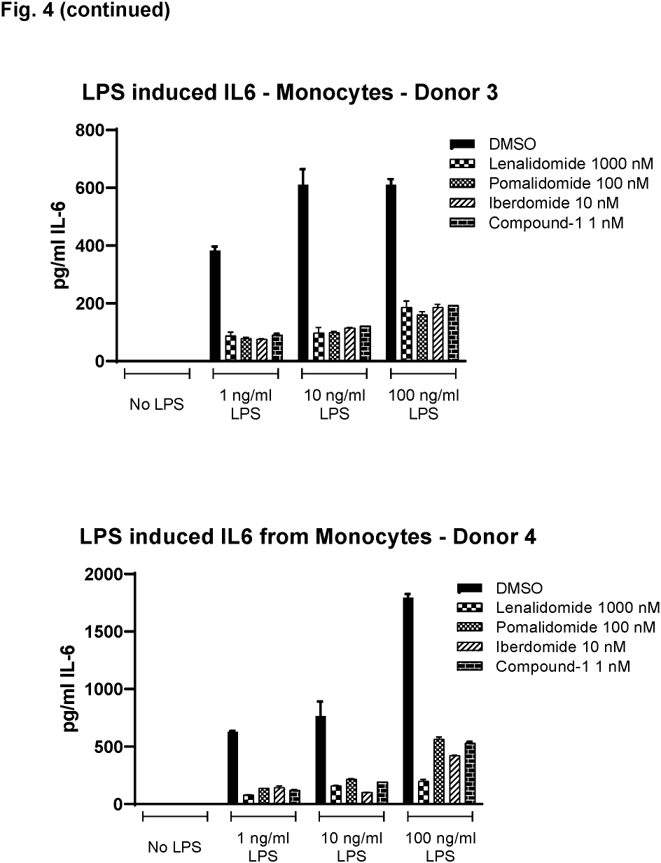

The data shows that LPS-induced IL-6 secretion from monocytes is diminished by pre-treatment with lenalidomide, pomalidomide, iberdomide or Compound 1 (see FIG. 4).

### Example 2: IL-6 Secretion From Monocytes Is Reduced by Compound 1 at a Range of Concentrations

Monocytes from two healthy human donors were isolated as in Example 1. Here isolated monocytes (1×10⁶ cells/ml) were treated with various concentrations of Compound 1 (1 nM, 10 nM, or 100 nM), or control DMSO (0.001%), overnight (~17 hours) before stimulation with various concentrations of LPS.

After 4 hours, 5 µg/ml of nigericin was added to activate the inflammasome for 1 hour, supernatants were harvested, and MSD analysis was performed to quantify secreted IL-6.

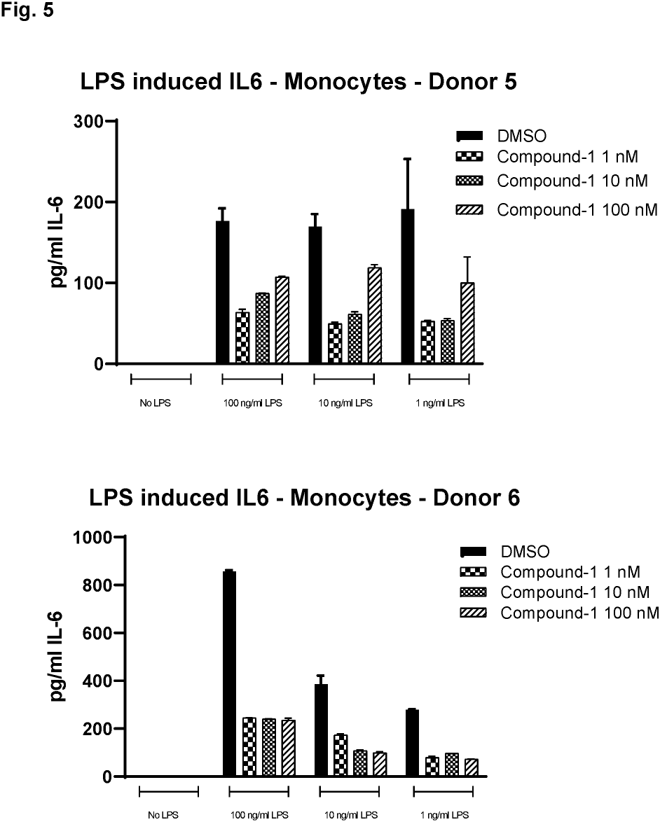

The data shows that LPS-induced IL-6 secretion from monocytes is diminished by pre-treatment with 1 nM, 10 nM or 100 nM Compound 1 (see FIG. 5).

### Example 3: Treatment With Lenalidomide, Pomalidomide, Iberdomide or Compound 1 Reduces IL-6 Secretion from Isolated Macrophages

Peripheral blood mononuclear cells (PBMCs) were isolated from the blood of two healthy human donors by Ficoll cell separation, and then monocytes were isolated from these PBMCs by positive selection using MACS C14 magnetic microbeads. The isolated monocytes were seeded at a concentration of 3×10⁵ cells/ml and incubated for 4 days with RPMI 1640 media containing M-CSF (50 ng/mL) (50% media replaced after 2 days) to obtain naive macrophages (M0). At the end of day 4, the macrophages were incubated overnight (~17 hours) with 1000 nM lenalidomide, 100 nM pomalidomide, 10 nM iberdomide or 1 nM Compound 1, or control DMSO (0.001%), before stimulation with various concentrations of LPS for IL-6 secretion.

After 6 hours, the cell culture was spun down at 300 g for 5 minutes to remove cells, and the supernatant was subjected to MSD analysis to quantify secreted IL-6.

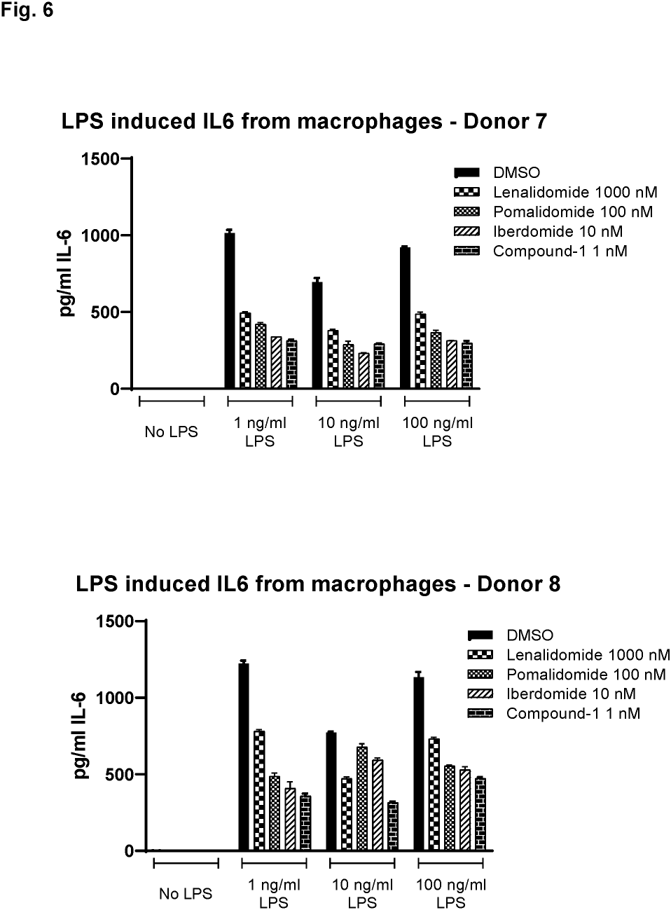

The data shows that LPS-induced IL-6 secretion from macrophages is diminished by pre-treatment with lenalidomide, pomalidomide, iberdomide or Compound 1 (see FIG. 6).

### Example 4: Treatment With Certain IMiD Compounds Reduces LPS-Induced Secretion of Proinflammatory Cytokines

Peripheral blood mononuclear cells (PBMCs) were isolated from the blood of healthy human donors by Ficoll cell separation. The PBMCs were seeded at a concentration of 2×10⁶ cells/ml and treated for 1 hour at 37° C. with various concentrations of IMiD compounds or control DMSO (0.25%), before stimulation with 1 ng/ml LPS for 18 hours at 37° C.

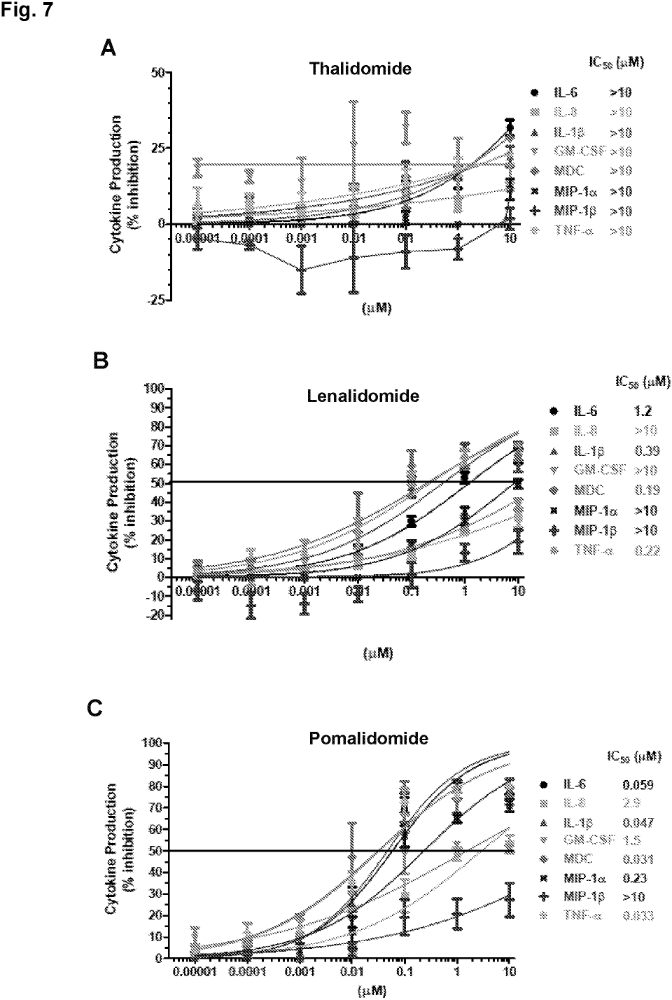

Supernatant samples were subjected to multiplex cytokine analysis (Luminex IS100 instrument) to quantify secreted levels of IL-6, IL-8, IL1-beta (IL-1β), GM-CSF, MDC, MIP-1alpha (MIP-1α), MIP-1beta (MIP-1β) and TNF-alpha (TNF-α) (see e.g. FIGS. 7A-C).

IC50 (half maximal inhibitory concentration) values were calculated to measure the potency of each IMiD compound in inhibiting LPS-stimulated secretion of each cytokine (see Table 25).

The data shows that LPS-induced secretion of proinflammatory cytokines from PBMCs is inhibited by pre-treatment with pomalidomide, lenalidomide, avadomide, iberdomide, Compound A or Compound B. Compounds A and B are IMiD compounds and regioisomers with the following structure:

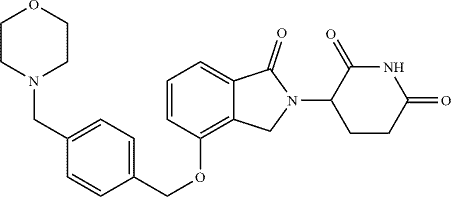 [SMILES: O=C1CCC(N2Cc3c(OCc4ccc(CN5CCOCC5)cc4)cccc3C2=O)C(=O)N1]

Thalidomide had minimal to no effect on IL-6, IL-8, IL-1β, GM-CSF, MDC, MIP-1α, MLP-1β and TNF-α secretion. In contrast, the thalidomide derivatives inhibited secretion of IL-6, IL-8, IL-1β, GM-CSF, MDC, MIP-1α and TNF-α with varied potencies.

TABLE 25

Summary of cytokine inhibitory profile of IMiD compounds

| IMiD | IC50 (µM) of cytokine production in LPS-stimulated PBMC | | | | | | | |
|---|---|---|---|---|---|---|---|---|
| | IL-6 | IL-8 | IL-1β | GM-CSF | MDC | MIP-1α | MIP-1β | TNF-α |
| Thalidomide | >10 | >10 | >10 | >10 | >10 | >10 | >10 | >10 |
| Pomalidomide | 0.059 | 2.9 | 0.047 | 1.5 | 0.031 | 0.23 | >10 | 0.033 |
| Lenalidomide | 1.2 | >10 | 0.39 | >10 | 0.19 | >10 | >10 | 0.22 |
| Avadomide | 0.060 | >10 | 0.054 | 0.95 | 0.062 | 0.30 | >10 | 0.034 |
| Iberdomide | 0.0038 | >10 | 0.00046 | 0.0022 | 0.0021 | 0.028 | >10 | 0.00059 |
| Compound A | 0.01 | >10 | 0.00085 | 0.0092 | 0.0026 | 0.19 | >10 | 0.0018 |
| Compound B | 0.083 | >10 | 0.0062 | 0.039 | 0.012 | 0.45 | >10 | 0.0095 |

### Example 5: IMiD/CELMoD-Mediated Suppression of LPS-Induced IL-6, TNF-Alpha and IL1-Beta from PBMCs

Fresh peripheral blood mononuclear cells (PBMCs) were isolated from the blood (buffy coat) of healthy human volunteers by Ficoll based cell separation. The isolated PBMCs were seeded at a concentration of 1×10⁶ cells/ml and treated with 1000 nM lenalidomide, 100 nM pomalidomide, 10 nM iberdomide, 1 nM Compound 1 (CC-92480) or control DMSO, overnight (~17 hours). The next morning, the PBMCs were stimulated with various concentrations of LPS (Lipopolysaccharides from Escherichia coli O111:B4).

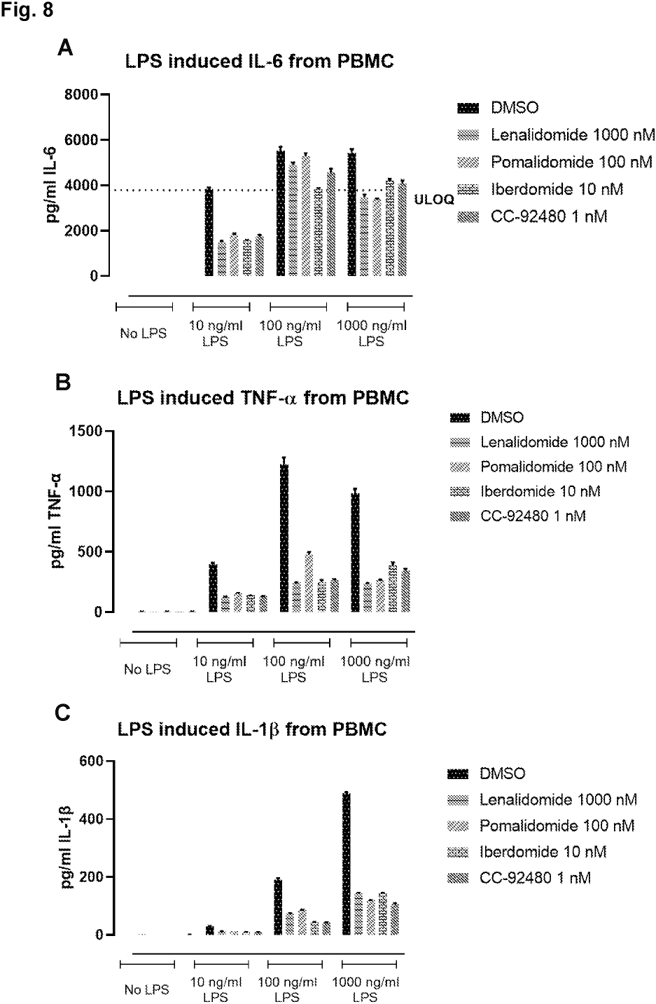

After 24 hours, the cell culture media was collected from the control and stimulated cells, spun down at 300 g for 5 minutes to remove cells and the supernatant was subjected to cytokine analysis to quantify secreted levels of IL-6, TNF-alpha (TNF-α) and IL1-beta (IL-1β) using an MSD assay (see e.g. FIGS. 8A-C).

The data shows that pre-treatment with lenalidomide, pomalidomide, iberdomide or Compound 1 reduces LPS-induced secretion of IL-6, TNF-α and IL-1β from PBMCs. Accordingly, pre-treatment with IMiDs/CELMoDs may be used as a single agent to target all three cytokines, rather than separate agents that each target a single cytokine, which is the current standard of care to manage CRS.

### Example 6: IMiD/CELMoD Mediated Suppression of CC-93269-Induced IL-6 From PBMCs with BCMA Expressing Target Cells

Fresh PBMCs were isolated from the blood (buffy coat) of healthy human volunteers by Ficoll based cell separation. The percentage of CD3+ T-cells (total) in the PBMCs was quantified by flow cytometry. The isolated PBMCs were seeded in 12-well plates at a concentration of 1.5×10⁶ cells/ml and treated with 1 nM Compound 1 (CC-92480), 1000 nM lenalidomide, 100 nM pomalidomide, 10 nM iberdomide, or control DMSO overnight (~17 hours).

After overnight incubation, target K562-BCMA cells (overexpress surface BCMA at a very high level) or control K562-MCB cells (no BCMA expression) were added to the wells at a ratio of 5:1, T-cells to target cells. K562-BCMA and K562-MCB are an isogenic pair that are derived from a chronic myelogenous leukemia (CML,) cell line. CC-93269 was added to the co-culture at various concentrations and incubated. CC-93269 is an anti-BCMA anti-CD3 bispecific T cell-engaging antibody and is also referred to herein as 42-TCBcv.

At 6, 24 and 48 hours post-incubation, the cell culture media was collected from all samples, spun down at 300 g for 5 minutes to remove cells and the supernatant was subjected to cytokine analysis to quantify secreted levels of IL-6 using an MSD assay.

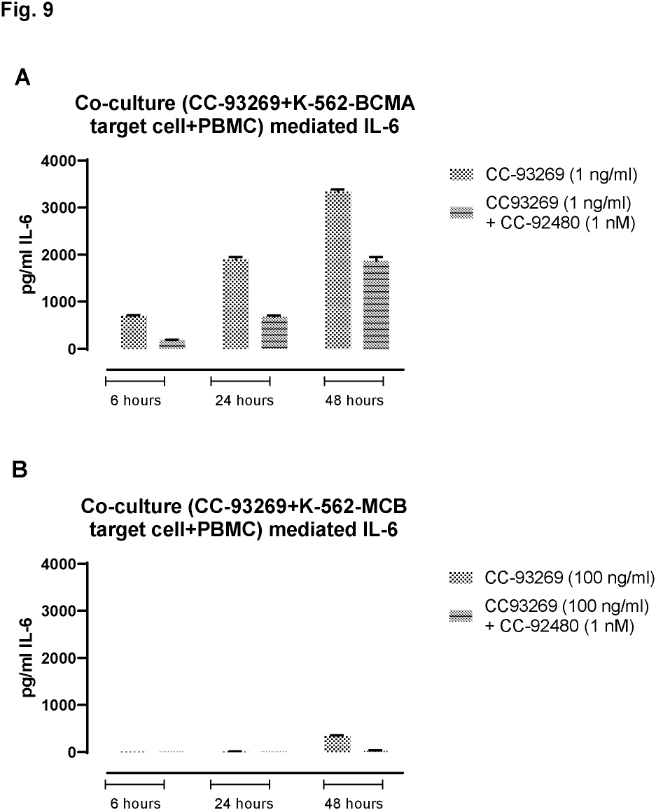

FIGS. 9A and B show data for Compound 1 pre-treatment of K562-BCMA and K562-MCB cocultures, respectively. IL-6 levels in the K562-BCMA co-culture is associated with CC-93269 binding to BCMA and CD3 and subsequent T-cell activation, while background IL-6 levels are shown in the K562-MCB co-culture.

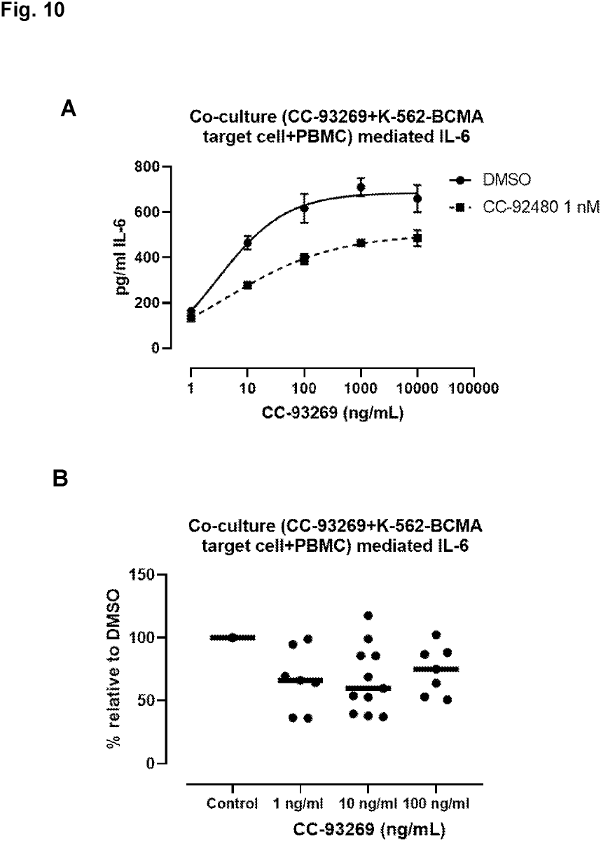

FIG. 10A shows the IL-6 levels at 24 hours for a K562-BCMA sample where PBMCs from an independent healthy donor were pre-treated with Compound 1 or control DMSO, and CC-93269 concentrations up to 10,000 ng/mL. The linear range of this in vitro system is lost at around 100 ng/mL CC-93269, but the data shows that even beyond the dynamic range, IL-6 secretion is suppressed at CC-93269 concentrations up to 10,000 ng/ml.

Independent data for PBMCs from 15 healthy donors was also collected. The PBMCs were pre-treated with Compound 1 or DMSO (control), and target cells (K562-BCMA) and CC-93269 added, as described above. FIG. 10B shows the IL-6 levels at 24 hours for the PBMCs pre-treated with Compound 1 normalized relative to the control treatment, with the control set at 100%. Overall, a median reduction of about two-fold in IL-6 levels was observed at 10 ng/ml CC-93269 across all donors tested. The circles indicate independent donors and the bars indicate median values; not all donors are shown because a data cut-off for the bottom ~10% of IL-6 concentrations, below 200 pg/ml, was used to eliminate small changes that could skew the result and to increase confidence in results.

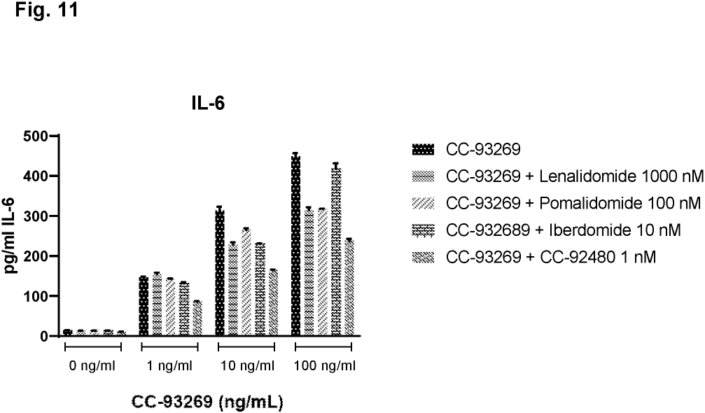

FIG. 11 shows the IL-6 levels at 24 hours in a co-culture of target K562-BCMA cells and PBMCs from one donor, pre-treated with 1000 nM lenalidomide, 100 nM pomalidomide, 10 nM iberdomide, 1 nM Compound 1 (CC-92480), or control DMSO. A reduction of CC-93269-mediated IL-6 secretion was also observed for lenalidomide, pomalidomide and iberdomide pre-treatment of PBMCs from two more healthy donors. The data shows that pre-treatment of a co-culture of PBMCs and BCMA-expressing cells (K562-BCMA) with Compound 1, lenalidomide, pomalidomide or iberdomide leads to diminished/attenuated secretion of IL-6 associated with CC-93269 activity.

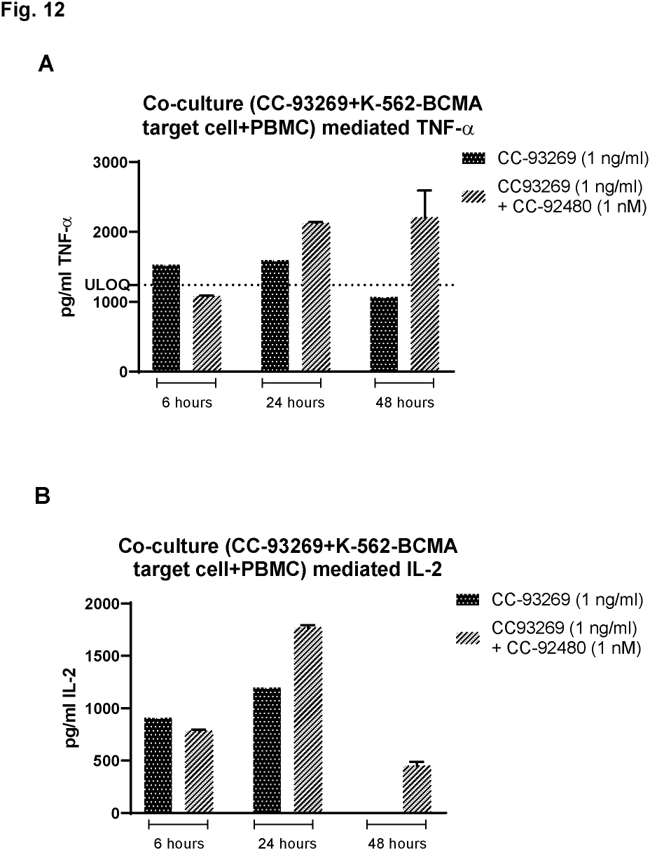

Notably, TNF-alpha, IFN-gamma and IL-2, critical cytokines related to T-cell lytic activity and immune activation, were also assessed in this in vitro model. Levels of TNF-alpha, IFN-gamma and IL-2 were quantified in the supernatant at 6, 24 and 48 hours post-incubation using the MSD assay, as for IL-6. FIGS. 12A-B shows that pre-treatment with Compound 1 (CC-92480) potentiates CC-93269-induced TNF-alpha and IL-2 secretion from PBMCs with K562 target cells at 24 hours. IFN-gamma was not impacted by pre-treatment with the IMiD/CELMoDs (data not shown).

### Example 7: Compound 1 (CC-92480)-Mediated Suppression of CC-93269-Induced IL1-Beta from PBMCs with BCMA Expressing Target Cells

Fresh PBMCs were isolated from the blood (buffy coat) of healthy human volunteers by Ficoll based cell separation. The percentage of CD3+ T-cells (total) in the PBMCs was quantified by flow cytometry. The isolated PBMCs were seeded in 12 well plates at a concentration of 1.5×10⁶ cells/ml and treated with 1 nM Compound 1 (CC-92480) or control DMSO overnight (~17 hours). After overnight incubation, target K562-BCMA cells were added to the wells at a ratio of 5:1, T-cells to target cells, followed by incubation with CC-93269 at various concentrations. K562-BCMA is a chronic myelogenous leukemia (CML) cell line which overexpress surface BCMA at a very high level.

At 6, 24 and 48 hours post-incubation, the cell culture media was collected from all samples, spun down at 300 g for 5 minutes to remove cells and the supernatant was subjected to cytokine analysis to quantify secreted levels of IL1-beta using an MSD assay.

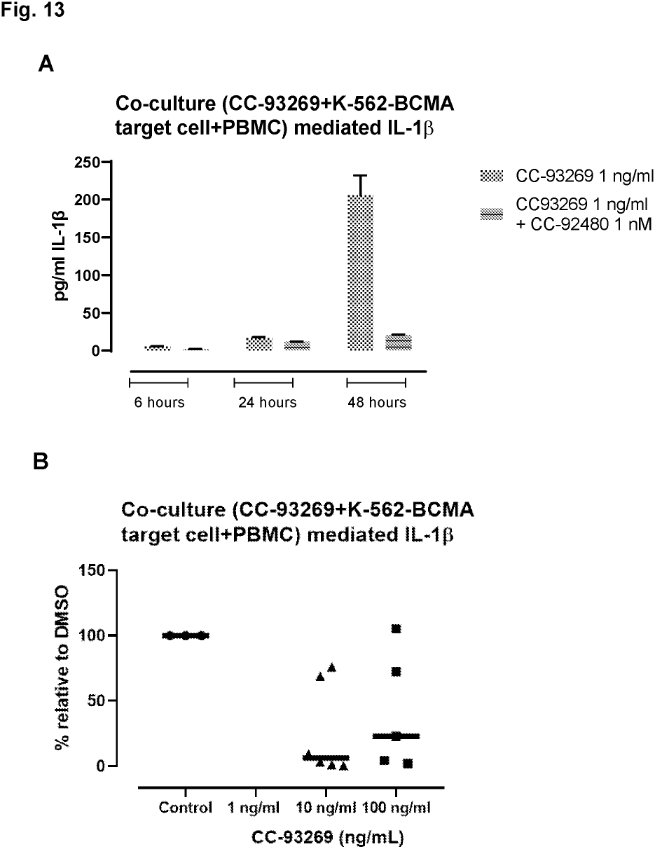

FIG. 13A shows that pre-treatment of a co-culture of PBMCs and BCMA-expressing cells (K562-BCMA) with Compound 1 leads to diminished/attenuated secretion of IL1-beta associated with CC-93269 activity (i.e. binding to BCMA and CD3 and subsequent T-cell activation). Taking into account data from the preceding examples, other IMIDs/CELMoDs such as lenalidomide, pomalidomide and iberdomide are expected to perform similar to Compound 1.

Independent data for PBMCs collected from 15 healthy donors was also collected. The PBMCs were pre-treated with Compound 1 or DMSO (control), and target cells (K562-BCMA) and CC-93269 added, as described above. FIG. 13B shows the IL1-beta levels at 24 hours for the PBMCs pre-treated with Compound 1 normalized relative to the control treatment, with the control set at 100%. Overall, there is a median ten-fold reduction in IL1-beta levels at 10 ng/ml CC-93269 across all donors tested.

### Example 8: Compound 1 (CC-92480)-Mediated Suppression of CC-93269-Induced IL-6 from PBMCs with Multiple Myeloma Cells

Fresh PBMCs were isolated from the blood (buffy coat) of human volunteers by Ficoll based cell separation. The percentage of CD3+ T-cells (total) in the PBMCs was quantified by flow cytometry. The isolated PBMCs were seeded in 12 well plates at a concentration of 1.5×10⁶ cells/ml and treated with 1 nM Compound 1 (CC-92480) or control DMSO overnight (~17 hours). After overnight incubation, the cells were washed to remove CC-92480, before target H929 cells were added to the wells at a ratio of 5:1, T-cells to target cells. H929 is a multiple myeloma cell line which expresses moderate levels of BCMA (four-fold lower than K562-BCMA).

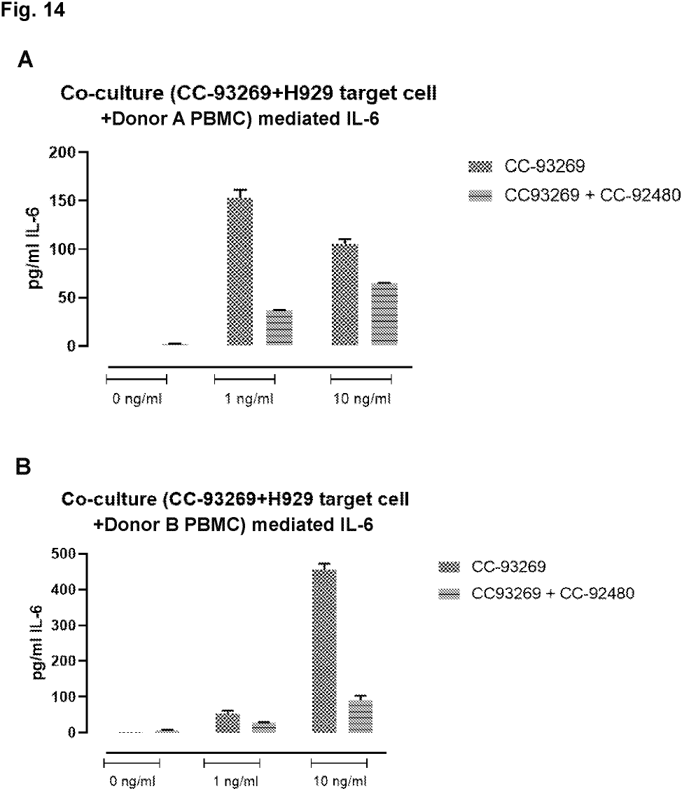

The co-culture was then incubated with CC-93269 at various concentrations. At 6, 24 and 48 hours post-incubation, the cell culture media was collected from all samples, spun down at 300 g for 5 minutes to remove cells and the supernatant was subjected to cytokine analysis to quantify secreted levels of IL-6 using an MSD assay. FIGS. 14A-B show data from two independent healthy donors.

The data shows that pre-treatment a co-culture of PBMCs and multiple myeloma cells with Compound 1 leads to diminished/attenuated secretion of IL-6 associated with CC-93269 activity (i.e. binding to BCMA and CD3 and subsequent T-cell activation). Taking into account data from other examples, other IMIDs/CELMoDs such as lenalidomide, pomalidomide and iberdomide are expected to perform similar to Compound 1.

### Example 9: IMiDs/CELMoDs Enhance CC-93269-Mediated T Cell Killing of MM Cells In Vitro

To evaluate the effects of Cereblon modulating (CM) agents on T-cell function after chronic stimulation (i.e. mimicking T cell exhaustion), CD3+ enriched healthy donor T-cells were stimulated with anti-CD3/CD28 in the presence of DMSO (control), 100 nM pomalidomide, 10 nM iberdomide (CC-220) or 1 nM Compound 1 (CC-92480) for 7 days. Prolonged and repetitive anti-CD3/CD28 stimulation of T-cells results in functional T-cell exhaustion in vitro.

The chronically stimulated T-cells were then used in CC-93269 mediated cytotoxicity assays with NCI-H929 or OPM-2 multiple myeloma (MM) target cells. The T-cells were washed to remove compounds, and mixed with fluorescently labelled MM cell lines (NCI-H929, OPM-2) at optimized effector-to-target (E:T) ratios (1:2 or 1:4) in the presence of fixed concentrations of CC-93269 (47 pM). Freshly thawed T-cells (i.e. non-exhausted T-cells) from the same donor were used as control effector cells. In this experimental cytotoxicity assay setup, the number of target cells was continuously monitored using the IncuCyte® S3 Live-Cell Analysis System for a minimum of 10 days. Cell culture supernatants were taken from wells from subsequent CC-93269 mediated cytotoxicity assays at 3 days after mixing with MM target cells. CD3+ T cells from 2 or 3 independent healthy donors were tested for each MM cell line.

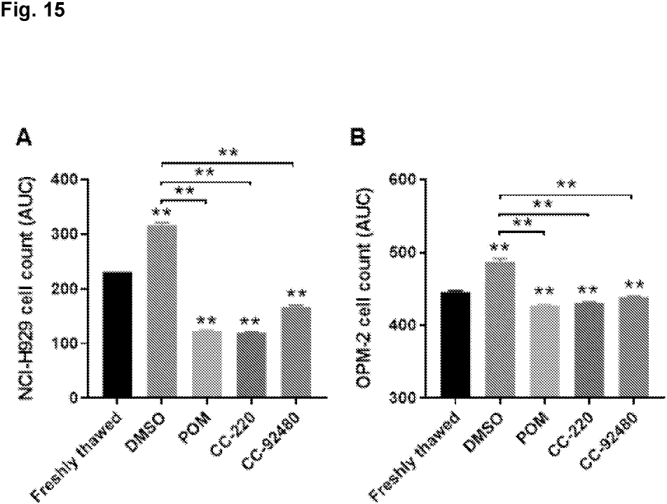

The results observed with both NCI-H929 (FIG. 15A) and OPM-2 cells (FIG. 15B) as MM target cells suggest that the tested CM agents (pomalidomide, iberdomide or CC-92480) had beneficial effects on the cytolytic function of T-cells in the context of bispecific antibody-directed target cell killing. T cells that had undergone chronic stimulation in the presence of DMSO (control) showed a delayed loss of bispecific antibody-induced cytolytic activity (i.e. functional T cell exhaustion), with regrowth of MM target cells over time. In contrast, T-cells that had undergone chronic stimulation in the presence of CM agents maintained CC-93269-induced cytolytic activity, comparable to the activity mediated by freshly thawed T-cells. Thus, T-cell exhaustion by chronic anti-CD3/CD28 stimulation was prevented by exposure to CM agents, resulting in preservation of cytolytic activity of effector cells in subsequent CC-93269 assays.

1. A cytokine inhibitor for use in a method of treating or preventing a cytokine-related adverse event or disease in a subject, wherein the cytokine inhibitor is pomalidomide, iberdomide, lenalidomide, avadomide or Compound 1, and wherein Compound 1 is 4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, an isotopolog, or a pharmaceutically acceptable salt thereof.

2. The cytokine inhibitor for use according to claim 1, wherein the cytokine-related adverse event or disease is cytokine release syndrome (CRS).

3. The cytokine inhibitor for use according to claim 1 or 2, wherein the subject has received or will receive a therapeutic agent that has caused or is likely to cause CRS.

4. The cytokine inhibitor for use according to claim 3, wherein the therapeutic agent is directed to BCMA (“BCMA therapeutic agent”).

5. The cytokine inhibitor for use according to claim 1, wherein the cytokine-related adverse event or disease is Coronavirus disease 2019 (COVID-19) or cytokine-mediated neurotoxicity.

6. A BCMA therapeutic agent for use in a method of treating a disorder associated with BCMA expression in a subject, wherein the method comprises:
a) administering to the subject the BCMA therapeutic agent, wherein the administering is likely to cause or has caused CRS in the subject; and
b) administering to the subject a cytokine inhibitor at a dose to prevent or reduce the development of CRS in the subject, wherein the cytokine inhibitor is pomalidomide, iberdomide, lenalidomide, avadomide or Compound 1, and wherein Compound 1 is 4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, an isotopolog or a pharmaceutically acceptable salt thereof.

7. A BCMA therapeutic agent for use in a method of treating a disorder associated with BCMA expression in a subject, wherein the method comprises:
a) administering to the subject a cytokine inhibitor (e.g. IL-6 inhibitor), wherein the cytokine inhibitor (e.g. IL-6 inhibitor) is pomalidomide, iberdomide, lenalidomide, avadomide or Compound 1, and wherein Compound 1 is 4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, an isotopolog or a pharmaceutically acceptable salt thereof; and
b) following administration of the cytokine inhibitor, administering to the subject the BCMA therapeutic agent, wherein the administering is likely to cause CRS in the subject.

8. The BCMA therapeutic agent for use according to claim 7, wherein the cytokine inhibitor (e.g. IL-6 inhibitor) is administered as a first dose about 7 days before the BCMA therapeutic agent.

9. A BCMA therapeutic agent for use in a method of treating a disorder associated with BCMA expression in a subject, wherein the method comprises:
a) administering to the subject the BCMA therapeutic agent, wherein the administering is likely to cause or has caused CRS in the subject; and
b) following administration of the BCMA therapeutic agent, administering to the subject a cytokine inhibitor (e.g. IL-6 inhibitor), wherein the cytokine inhibitor (e.g. IL-6 inhibitor) is pomalidomide, iberdomide, lenalidomide, avadomide or Compound 1, and wherein Compound 1 is 4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, an isotopolog or a pharmaceutically acceptable salt thereof.

10. The BCMA therapeutic agent for use according to claim 9, wherein the BCMA therapeutic agent is administered as a first dose about 7 days before the cytokine inhibitor.

11. The BCMA therapeutic agent for use according to any one of claims 6 to 10, wherein the disorder associated with BCMA expression in a subject is: multiple myeloma, optionally wherein the multiple myeloma is high-risk multiple myeloma or relapsed and refractory multiple myeloma; chronic lymphocytic leukemia; or a non-Hodgkins lymphoma, optionally wherein the non-Hodgkin’s lymphoma is Burkitt’s lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma.

12. The cytokine inhibitor for use according to claim 3 or 4, or the BCMA therapeutic agent for use according to any one of claims 6 to 11, wherein the therapeutic agent that has caused or is likely to cause CRS or the BCMA therapeutic agent is a T cell engager.

13. The cytokine inhibitor for use according to claim 12, or the BCMA therapeutic agent for use according to claim 12, wherein the T cell engager is a multispecific antibody which specifically binds to a cancer antigen (e.g. BCMA) and to an antigen that promotes activation of one or more T cells, optionally wherein the antigen that promotes activation of one or more T cells is selected from the group consisting of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226, preferably wherein the antigen that promotes activation of one or more T cells is CD3.

14. The cytokine inhibitor for use according to claim 13, or the BCMA therapeutic agent for use according to claim 13, wherein the multispecific antibody is a bispecific antibody, optionally wherein the bispecific antibody comprises two Fab fragments of an anti-BCMA antibody, one Fab fragment of an anti-CD3 antibody, and one Fc portion and the bispecific antibody is in the format BCMA Fab - Fc - CD3 Fab - BCMA Fab.

15. The cytokine inhibitor for use according to claim 12, or the BCMA therapeutic agent for use according to claim 12, wherein the T cell engager is a chimeric antigen receptor (CAR) directed to a cancer antigen (e.g. BCMA), or a T cell expressing at least one CAR directed to a cancer antigen (e.g. BCMA).

16. The cytokine inhibitor for use according to claim 4 or any one of claims 12 to 14 when dependent on claim 4, or the BCMA therapeutic agent for use according to any one of claims 6 to 15, wherein the BCMA therapeutic agent comprises a CDR1H, CDR2H, CDR3H, CDR1L, CDR2L, and CDR3L region combination selected from:
a) CDR1H region of SEQ ID NO:21, CDR2H region of SEQ ID NO:22, CDR3H region of SEQ ID NO: 17, CDR1L region of SEQ ID NO:27, CDR2L region of SEQ ID NO:28, and CDR3L region of SEQ ID NO:20, optionally wherein the BCMA therapeutic agent comprises a VH of SEQ ID NO: 10 and a VL of SEQ ID NO: 14;
b) CDR1H region of SEQ ID NO:21, CDR2H region of SEQ ID NO:22, CDR3H region of SEQ ID NO: 17, CDR1L region of SEQ ID NO:25, CDR2L region of SEQ ID NO:26, and CDR3L region of SEQ ID NO:20, optionally wherein the BCMA therapeutic agent comprises a VH of SEQ ID NO: 10 and a VL of SEQ ID NO: 13; and
c) CDR1H region of SEQ ID NO: 15, CDR2H region of SEQ ID NO: 16, CDR3H region of SEQ ID NO: 17, CDR1L region of SEQ ID NO: 18, CDR2L region of SEQ ID NO: 19, and CDR3L region of SEQ ID NO:20, optionally wherein the BCMA therapeutic agent comprises a VH of SEQ ID NO: 9 and a VL of SEQ ID NO: 11.

17. The cytokine inhibitor for use according to claim 13 or 14, or the BCMA therapeutic agent for use according to claim 13 or 14, wherein the multispecific antibody comprises a heavy and light chain set of the polypeptides set forth in SEQ ID NO:48, SEQ ID NO:55, SEQ ID NO: 56, and two copies of SEQ ID NO: 57.

18. The cytokine inhibitor for use according to claim 4 or any one of claims 12 to 15 when dependent on claim 4, or the BCMA therapeutic agent for use according to any one of claims 6 to 15, wherein the BCMA therapeutic agent comprises a VH comprising a CDR1H of SEQ ID NO:64, a CDR2H of SEQ ID NO:65 a CDR3H of SEQ ID NO:66, and a VL comprising a CDR1L, a CDR2L and a CDR3L set of sequences selected from:
a) CDR1L of SEQ ID NO:67, CDR2L of SEQ ID NO:68, and CDR3L of SEQ ID NO:69, optionally wherein the BCMA therapeutic agent comprises a VH of SEQ ID NO:76 and a VL of SEQ ID NO:77;
b) CDR1L of SEQ ID NO:70, CDR2L of SEQ ID NO:71, and CDR3L of SEQ ID NO:72, optionally wherein the BCMA therapeutic agent comprises a VH of SEQ ID NO:76 and a VL of SEQ ID NO:78; or
c) CDR1L of SEQ ID NO:73, CDR2L of SEQ ID NO:74, and CDR3L of SEQ ID NO:75 optionally wherein the BCMA therapeutic agent comprises a VH of SEQ ID NO:76 and a VL of SEQ ID NO:79.

19. The cytokine inhibitor for use according to any one of claims 3, 4, or 12 to 18, or the BCMA therapeutic agent for use according to any one of claims 6 to 18, wherein:
(a) the cytokine inhibitor is administered before (e.g. within 12 or 24 hours before) administration of the therapeutic agent (e.g. BCMA therapeutic agent);
(b) the cytokine inhibitor is administered on the same day as administration of the therapeutic agent (e.g. BCMA therapeutic agent);
(c) the cytokine inhibitor is administered after (e.g. within 12 or 24 hours after) administration of the therapeutic agent (e.g. BCMA therapeutic agent); or
(d) the cytokine inhibitor is administered within 12 hours after diagnosis of CRS.

20. The cytokine inhibitor or the BCMA therapeutic agent for use according to any one of the preceding claims , wherein the cytokine inhibitor is a proinflammatory cytokine inhibitor.

21. The cytokine inhibitor or the BCMA therapeutic agent for use according to any one of the preceding claims , wherein the cytokine-related adverse event or disease is not breast cancer.
